SEQUENCE LISTING (880 sequences)

SEQ ID NO: 1 - DNA, 51 bp, Homo sapiens
  allele (26)...(0): single nucleotide polymorphism
cgctgacaggggagtctgagccacagacccgctcacccgagtgcacgcacg

SEQ ID NO: 2 - DNA, 51 bp, Homo sapiens
  allele (26)...(0): single nucleotide polymorphism
atggatagtccatctggttggatgctgtgtactcgttggcctcgttcaggt

SEQ ID NO: 3 - DNA, 51 bp, Homo sapiens
  allele (26)...(0): single nucleotide polymorphism
aatacaaagctgagtggagagcagtgggtgaagaagtatggcattccaagt

SEQ ID NO: 4 - DNA, 51 bp, Homo sapiens
  allele (26)...(0): single nucleotide polymorphism
tattgttattatgtattctgtttacgtgtttctgtgtcactgctaagagaa

SEQ ID NO: 5 - DNA, 51 bp, Homo sapiens
  allele (26)...(0): single nucleotide polymorphism
caaggagcattgaccgtgagaaatatgaacagtttgcgttatatggctatg

SEQ ID NO: 6 - DNA, 51 bp, Homo sapiens
  allele (26)...(0): single nucleotide polymorphism
tggcgtcgtattttgggcattcagtcgctgtcactgacgtcaacggggatg

SEQ ID NO: 7 - DNA, 51 bp, Homo sapiens
  allele (26)...(0): single nucleotide polymorphism
agggggcctatgaagcagagctggcggtgcacctgccccagggcgcccact

SEQ ID NO: 8 - DNA, 51 bp, Homo sapiens
  allele (26)...(0): single nucleotide polymorphism
gttactgtgaagcgggcttcagctcggtggtcactcaggccggagagctgg

SEQ ID NO: 9 - DNA, 51 bp, Homo sapiens
  allele (26)...(0): single nucleotide polymorphism
gtgacaagtacttcatagaggatggtcgcctggtcatccacagcctggact

SEQ ID NO: 10 - DNA, 51 bp, Homo sapiens
  allele (26)...(0): single nucleotide polymorphism
aaggagaaaacaatgaagaaccgaatgaagacgaagactctgaggctgaga

SEQ ID NO: 11 - DNA, 51 bp, Homo sapiens
  allele (26)...(0): single nucleotide polymorphism
aaaacaatgaagaaccgaacgaagatgaagactctgaggctgagaatacca

SEQ ID NO: 12 - DNA, 51 bp, Homo sapiens
  allele (26)...(0): single nucleotide polymorphism
agaacggccagcccctgtggatcctgggggatgtcttcctcaggtcctact

SEQ ID NO: 13 - DNA, 51 bp, Homo sapiens
  allele (26)...(0): single nucleotide polymorphism
ggactccagtgtccagggcatccagttacatcctatcctggcggccactca

SEQ ID NO: 14 - DNA, 51 bp, Homo sapiens
  allele (26)...(0): single nucleotide polymorphism
taagacgggcagctacacccgcagcggttacctgccagctgagcaactggt

SEQ ID NO: 15 - DNA, 51 bp, Homo sapiens
  allele (26)...(0): single nucleotide polymorphism
tccagcccaaggccaattttgatgcgcagcagtttgcagggacctggctcc

SEQ ID NO: 16 - DNA, 51 bp, Homo sapiens
  allele (26)...(0): single nucleotide polymorphism
aggtaggagggcttggtctccaaacgcctattgtttcattctccacagtgc

SEQ ID NO: 17 - DNA, 51 bp, Homo sapiens
  allele (26)...(0): single nucleotide polymorphism
aacagccggcagatgtaactggtaccgccttgcccagggtgggccccgtga

SEQ ID NO: 18 - DNA, 51 bp, Homo sapiens
  allele (26)...(0): single nucleotide polymorphism
tccagcgccgggcaggaggggtcctagttgcctcccatctgcagagcttcc

SEQ ID NO: 19 - DNA, 51 bp, Homo sapiens
  allele (26)...(0): single nucleotide polymorphism
atgtgcccactgcattgggttgtccgggagttgatactggtgggatcacag

SEQ ID NO: 20 - DNA, 51 bp, Homo sapiens
  allele (26)...(0): single nucleotide polymorphism
cagaatatggaggcacaggagcttcttttttatccactgtgctcgtgatag

SEQ ID NO: 21 - DNA, 51 bp, Homo sapiens
  allele (26)...(0): single nucleotide polymorphism
gaataagaaattcaatctggatgcattggtgacccataccctgccttttga

SEQ ID NO: 22 - DNA, 51 bp, Homo sapiens
  allele (26)...(0): single nucleotide polymorphism
gtgctccagaggggccagcaggcacgggaaaaaccgaaaccaccaaggact

SEQ ID NO: 23 - DNA, 51 bp, Homo sapiens
  allele (26)...(0): single nucleotide polymorphism
cagaggggccagcaggcacaggaaagaccgaaaccaccaaggacttggcta

SEQ ID NO: 24 - DNA, 51 bp, Homo sapiens
  allele (26)...(0): single nucleotide polymorphism
agcaatgggaaagttttttaaaggattggcttcttctggtgcttgggcttg

SEQ ID NO: 25 - DNA, 51 bp, Homo sapiens
  allele (26)...(0): single nucleotide polymorphism
cttcttctggtgcttgggcttgctttgatgaattcaaccggattgagttgg

SEQ ID NO: 26 - DNA, 51 bp, Homo sapiens
  allele (26)...(0): single nucleotide polymorphism
agcggcccaccatggccctagggtcatcaacaagtccagcagcaatcatgg

SEQ ID NO: 27 - DNA, 51 bp, Homo sapiens
  allele (26)...(0): single nucleotide polymorphism
ccgatggctatgagcaggctgctcgtgttgctattgaacacctggacaaga

SEQ ID NO: 28 - DNA, 51 bp, Homo sapiens
  allele (26)...(0): single nucleotide polymorphism
caagttcctcaataaagtggcagttttcaggttctactggctccacttctc

SEQ ID NO: 29 - DNA, 51 bp, Homo sapiens
  allele (26)...(0): single nucleotide polymorphism
tcaccctcaggaggtggctgttctgtgtccacgacaactacagaaacaacc

SEQ ID NO: 30 - DNA, 51 bp, Homo sapiens
  allele (26)...(0): single nucleotide polymorphism
tcttcaacatcgtctattggctttattatgtgaactaaaacatggcctccc

SEQ ID NO: 31 - DNA, 51 bp, Homo sapiens
  allele (26)...(0): single nucleotide polymorphism
ggattttggacagactcctagatggttatgacaatcgcctgagaccaggat

SEQ ID NO: 32 - DNA, 51 bp, Homo sapiens
  allele (26)...(0): single nucleotide polymorphism
gggcccacttccccctggacgtccagtggaacgacctggactacatggact

SEQ ID NO: 33 - DNA, 51 bp, Homo sapiens
  allele (26)...(0): single nucleotide polymorphism
tgaacgagccttccaacttcatcagaggctctgaggacggctgccccaaca

SEQ ID NO: 34 - DNA, 51 bp, Homo sapiens
  allele (26)...(0): single nucleotide polymorphism
aattccaaatgagctctccaaccacatattttctgcgtttttgatccagac

SEQ ID NO: 35 - DNA, 51 bp, Homo sapiens
  allele (26)...(0): single nucleotide polymorphism
aattccagatgagctctccaaccacatattttctgcgtttttgatccagac

SEQ ID NO: 36 - DNA, 51 bp, Homo sapiens
  allele (26)...(0): single nucleotide polymorphism
ggaccatctctgtgaccacacctgcagacgctgtcattggccactactcgc

SEQ ID NO: 37 - DNA, 51 bp, Homo sapiens
  allele (26)...(0): single nucleotide polymorphism
acccctggaatagagaggatgctgtgttcctgaagaatgaggctcagcgca

SEQ ID NO: 38 - DNA, 51 bp, Homo sapiens
  allele (26)...(0): single nucleotide polymorphism
tgacgtcatccatgtccaatgtccataccatggcccccccaaaatgctctc

SEQ ID NO: 39 - DNA, 51 bp, Homo sapiens
  allele (26)...(0): single nucleotide polymorphism
gaagggatataactgaagcaataaatttttcacggttggcaaatgtggaca

SEQ ID NO: 40 - DNA, 51 bp, Homo sapiens
  allele (26)...(0): single nucleotide polymorphism
aggactgtttttcattcagcttcagcgtgattcccatgggctcttctgtga

SEQ ID NO: 41 - DNA, 51 bp, Homo sapiens
  allele (26)...(0): single nucleotide polymorphism
atgtctcaggattctacccaaagcctgtgtgggtgatgtggatgcggggtg

SEQ ID NO: 42 - DNA, 51 bp, Homo sapiens
  allele (26)...(0): single nucleotide polymorphism
tggcaataatagtgccttccttgcttcttttgctatgccttgcattatggt

SEQ ID NO: 43 - DNA, 51 bp, Homo sapiens
  allele (26)...(0): single nucleotide polymorphism
ctgtgatatctacatctgggcgcccttggccgggacttgtggggtccttct

SEQ ID NO: 44 - DNA, 51 bp, Homo sapiens
  allele (26)...(0): single nucleotide polymorphism
agggtctgcgacagggttactttgtagaagctcagcccaagattgtcctgg

SEQ ID NO: 45 - DNA, 51 bp, Homo sapiens
  allele (26)...(0): single nucleotide polymorphism
atggccagtgctgggtctttgctggagtgaccaccacagtgctgcgctgcc

SEQ ID NO: 46 - DNA, 51 bp, Homo sapiens
  allele (26)...(0): single nucleotide polymorphism
gctctgtggagtccatcaagaatgggctggtctacatgaagtacgacacgc

SEQ ID NO: 47 - DNA, 51 bp, Homo sapiens
  allele (26)...(0): single nucleotide polymorphism
gcatccagtgggtaggggaccctcgttggaaggatggctccattgtcatac

SEQ ID NO: 48 - DNA, 51 bp, Homo sapiens
  allele (26)...(0): single nucleotide polymorphism
tacacaacctagactacagtgacaacggcacgttcacttgtgacgtcaaaa

SEQ ID NO: 49 - DNA, 51 bp, Homo sapiens
  allele (26)...(0): single nucleotide polymorphism
agtcccttctccgtggcacctacgcctatggttttgagaagccctctgcca

SEQ ID NO: 50 - DNA, 51 bp, Homo sapiens
  allele (26)...(0): single nucleotide polymorphism
agtcttactttgccattaaccacaatcccgacgccaaggacttgaagcagc

SEQ ID NO: 51 - DNA, 51 bp, Homo sapiens
  allele (26)...(0): single nucleotide polymorphism
actttgccattaaccacaaccccgatgccaaggacttgaagcagctcgcgc

SEQ ID NO: 52 - DNA, 51 bp, Homo sapiens
  allele (26)...(0): single nucleotide polymorphism
tggagcgagcgtggatccagttcgctgcggggttgtttgggtcaagttgct

SEQ ID NO: 53 - DNA, 51 bp, Homo sapiens
  allele (26)...(0): single nucleotide polymorphism
gttaccagacgctggagctggagaaagagtttcactacaatcgctacctga

SEQ ID NO: 54 - DNA, 51 bp, Homo sapiens
  allele (26)...(0): single nucleotide polymorphism
tcaggtagcgattgtagtgaaattccttctccagctccagggtctggtagc

SEQ ID NO: 55 - DNA, 51 bp, Homo sapiens
  allele (26)...(0): single nucleotide polymorphism
gggaagcatttgccaatcagtccagggcggaaagggatgccttcctgcagg

SEQ ID NO: 56 - DNA, 51 bp, Homo sapiens
  allele (26)...(0): single nucleotide polymorphism
acagccagcggtttggcctgcaccatgtcaacttcagcgacagcagcaagt

SEQ ID NO: 57 - DNA, 51 bp, Homo sapiens
  allele (26)...(0): single nucleotide polymorphism
atctggtcaccctgcagaacctgggtgtgtcccactaccgtttttccatct

SEQ ID NO: 58 - DNA, 51 bp, Homo sapiens
  allele (26)...(0): single nucleotide polymorphism
tggtgtgggccttggtgaactctagaacgcggctaatgtctcctggtttgg

SEQ ID NO: 59 - DNA, 51 bp, Homo sapiens
  allele (26)...(0): single nucleotide polymorphism
ggaagctgactccagaggccatgcctgacctcaactcctccactgactctg

SEQ ID NO: 60 - DNA, 51 bp, Homo sapiens
  allele (26)...(0): single nucleotide polymorphism
gtagtgaggaacaagccagagctgtccagatgagtacaaaagtcctgatcc

SEQ ID NO: 61 - DNA, 51 bp, Homo sapiens
  allele (26)...(0): single nucleotide polymorphism
agttggaagtgaatggatcgcagcattcactgacctgtgcttttgaggacc

SEQ ID NO: 62 - DNA, 51 bp, Homo sapiens
  allele (26)...(0): single nucleotide polymorphism
taaatattcgagacattgacaagatttatgttcgaacaggtatctaccatg

SEQ ID NO: 63 - DNA, 51 bp, Homo sapiens
  allele (26)...(0): single nucleotide polymorphism
agatctttgaggaaggggaatctgatgatgagtttgacatggatgagaatc

SEQ ID NO: 64 - DNA, 51 bp, Homo sapiens
  allele (26)...(0): single nucleotide polymorphism
ttctgacgcacatgttttgtacatttcagaccaaggaaaacctcttttttg

SEQ ID NO: 65 - DNA, 51 bp, Homo sapiens
  allele (26)...(0): single nucleotide polymorphism
ttagtatcattcactgtgatctaaagcctgaaaatatccttctttgtaacc

SEQ ID NO: 66 - DNA, 51 bp, Homo sapiens
  allele (26)...(0): single nucleotide polymorphism
aggtatataccatcatgtacagttgctggcatgagaaagcagatgagcgtc

SEQ ID NO: 67 - DNA, 51 bp, Homo sapiens
  allele (26)...(0): single nucleotide polymorphism
tggctccggctacaccaacatcatgcgggtgctaagcatatcctgagacgc

SEQ ID NO: 68 - DNA, 51 bp, Homo sapiens
  allele (26)...(0): single nucleotide polymorphism
gcttgccaatttctcgtctgtatgccaagtactttcaaggagatctgaatc

SEQ ID NO: 69 - DNA, 51 bp, Homo sapiens
  allele (26)...(0): single nucleotide polymorphism
ctgtggagtacatgtagctgaagagtcgctcaatcttcctcaagggaacac

SEQ ID NO: 70 - DNA, 51 bp, Homo sapiens
  allele (26)...(0): single nucleotide polymorphism
acatcatattggcgctgctgacgggtgtactgccccctggcatgctagatg

SEQ ID NO: 71 - DNA, 51 bp, Homo sapiens
  allele (26)...(0): single nucleotide polymorphism
cagtgtagaaataggggtgctccatggcctctcttgcagtaagccgtgact

SEQ ID NO: 72 - DNA, 51 bp, Homo sapiens
  allele (26)...(0): single nucleotide polymorphism
agctcaatggtggctctgcgtgctcatcccgaagtgacctgcctggttccg

SEQ ID NO: 73 - DNA, 51 bp, Homo sapiens
  allele (26)...(0): single nucleotide polymorphism
aattcaacccactcatctatggcaacgatgtggattctgtggatgttgcaa

SEQ ID NO: 74 - DNA, 51 bp, Homo sapiens
  allele (26)...(0): single nucleotide polymorphism
agaccccgccgtcccctggccaagccgtggagtgctgccaaggggactggt

SEQ ID NO: 75 - DNA, 51 bp, Homo sapiens
  allele (26)...(0): single nucleotide polymorphism
ctcacgctttgcagtcatctggtccacctagcactccctcctctcctcggc

SEQ ID NO: 76 - DNA, 51 bp, Homo sapiens
  allele (26)...(0): single nucleotide polymorphism
ttaacacgagggagcctgtgatgctagcctgctatgtgtggggcttctatc

SEQ ID NO: 77 - DNA, 51 bp, Homo sapiens
  allele (26)...(0): single nucleotide polymorphism
cccctgtgatcaatatcacctggctgcgcaacggccaaactgtcactgagg

SEQ ID NO: 78 - DNA, 51 bp, Homo sapiens
  allele (26)...(0): single nucleotide polymorphism
caccaccagatgccatggagaccctagtctgtgccctgggcctggccatcg

SEQ ID NO: 79 - DNA, 51 bp, Homo sapiens
  allele (26)...(0): single nucleotide polymorphism
ccatggagaccctggtctgtgccctaggcctggccatcggcctggtgggct

SEQ ID NO: 80 - DNA, 51 bp, Homo sapiens
  allele (26)...(0): single nucleotide polymorphism
tggtctgtgccctgggcctggccattggcctggtgggcttcctcgtgggca

SEQ ID NO: 81 - DNA, 51 bp, Homo sapiens
  allele (26)...(0): single nucleotide polymorphism
tgggcctggccatcggcctggtggggttcctcgtgggcaccgtcctcatca

SEQ ID NO: 82 - DNA, 51 bp, Homo sapiens
  allele (26)...(0): single nucleotide polymorphism
tcggcctggtgggcttcctcgtgggtaccgtcctcatcatcatgggcacat

SEQ ID NO: 83 - DNA, 51 bp, Homo sapiens
  allele (26)...(0): single nucleotide polymorphism
gtttcctcattagccctgtgacccctgcacacgcagggacctacagatgtc

SEQ ID NO: 84 - DNA, 51 bp, Homo sapiens
  allele (26)...(0): single nucleotide polymorphism
ttgacatctaccatctatccagggaaggggaagcccatgaacttaggctcc

SEQ ID NO: 85 - DNA, 51 bp, Homo sapiens
  allele (26)...(0): single nucleotide polymorphism
cttctagtagttggccttcacccacagaaccaagcttcaaaactggtatcg

SEQ ID NO: 86 - DNA, 51 bp, Homo sapiens
  allele (26)...(0): single nucleotide polymorphism
ggtactcagtggccatcatcctctttaccatccttcccttctttctccttc

SEQ ID NO: 87 - DNA, 51 bp, Homo sapiens
  allele (26)...(0): single nucleotide polymorphism
tggccatcatcctcttcaccatcctccccttctttctccttcatcgctggt

SEQ ID NO: 88 - DNA, 51 bp, Homo sapiens
  allele (26)...(0): single nucleotide polymorphism
aggagctcaagcgtgaggccgagactctacgggagcgggaaggcgaggagt

SEQ ID NO: 89 - DNA, 51 bp, Homo sapiens
  allele (26)...(0): single nucleotide polymorphism
tcatgggcaacctaaggcacaagtgtgtgcgcaacttcacagcgctcaacg

SEQ ID NO: 90 - DNA, 51 bp, Homo sapiens
  allele (26)...(0): single nucleotide polymorphism
cggaatacctggccatcacctctgagagcaaagagaactgcacgggcgtcc

SEQ ID NO: 91 - DNA, 51 bp, Homo sapiens
  allele (26)...(0): single nucleotide polymorphism
agagtggcgagtgggtcatcgtggatgccgtgggcacctacaacaccagga

SEQ ID NO: 92 - DNA, 51 bp, Homo sapiens
  allele (26)...(0): single nucleotide polymorphism
acaacaccaggaagtacgagtgctgtgccgagatctacccggacatcacct

SEQ ID NO: 93 - DNA, 51 bp, Homo sapiens
  allele (26)...(0): single nucleotide polymorphism
agaggctctttctgcagaaacttcccaaattactttgcatgaaagatcatg

SEQ ID NO: 94 - DNA, 51 bp, Homo sapiens
  allele (26)...(0): single nucleotide polymorphism
gtctaggatggagatcctacaaacatgtcagtgggcagatgctgtattttg

SEQ ID NO: 95 - DNA, 51 bp, Homo sapiens
  allele (26)...(0): single nucleotide polymorphism
ataacttgcatgatcttgtcaaacagcttcatctgtactgcttgaatacat

SEQ ID NO: 96 - DNA, 51 bp, Homo sapiens
  allele (26)...(0): single nucleotide polymorphism
ttacgtcgccaaattcccagggcacgttgcgcacgaacttcagtacgggat

SEQ ID NO: 97 - DNA, 51 bp, Homo sapiens
  allele (26)...(0): single nucleotide polymorphism
tccctgtgacccaggcaggtgcatgggtgacactggtcgtgacctggccag

SEQ ID NO: 98 - DNA, 51 bp, Homo sapiens
  allele (26)...(0): single nucleotide polymorphism
cggcacacaggccgctcgccggagctgtggcccacccccagcccctggcca

SEQ ID NO: 99 - DNA, 51 bp, Homo sapiens
  allele (26)...(0): single nucleotide polymorphism
agaactcgcgggtctcccactacattatcaactcgctgcccaaccgccgtt

SEQ ID NO: 100 - DNA, 51 bp, Homo sapiens
  allele (26)...(0): single nucleotide polymorphism
ctgcaactaccttgaaccagttgagttgcggatccaccctcagcagcagcc

SEQ ID NO: 101 - DNA, 51 bp, Homo sapiens
  allele (26)...(0): single nucleotide polymorphism
cagcatgacctggcactgtacttcgaggaaagttggggatttcaccgtagt

SEQ ID NO: 102 - DNA, 51 bp, Homo sapiens
  allele (26)...(0): single nucleotide polymorphism
ttgcaacttgaggtcggtgcttagtatgagacagaagccattctgcagtgt

SEQ ID NO: 103 - DNA, 51 bp, Homo sapiens
  allele (26)...(0): single nucleotide polymorphism
tttacagttttcttactgcatcatctatgtcagaaatctgttccttcagct

SEQ ID NO: 104 - DNA, 51 bp, Homo sapiens
  allele (26)...(0): single nucleotide polymorphism
agagccactacaaggtggactactcgcgttttcacaagacctacgaggtgg

SEQ ID NO: 105 - DNA, 51 bp, Homo sapiens
  allele (26)...(0): single nucleotide polymorphism
tcacacctgtcctgaccctggaggacgggttctacgaagttgactacaaca

SEQ ID NO: 106 - DNA, 51 bp, Homo sapiens
  allele (26)...(0): single nucleotide polymorphism
gcaggatcacctgcaccctcttggggaccatgatgctcatccagctgtcta

SEQ ID NO: 107 - DNA, 51 bp, Homo sapiens
  allele (26)...(0): single nucleotide polymorphism
agtcagacaccagcttagaaatgactatgggcaatgccttgtttcttgatg

SEQ ID NO: 108 - DNA, 51 bp, Homo sapiens
  allele (26)...(0): single nucleotide polymorphism
ggaggacaggcaactcatcaccgaattagtcatcagcaagatgaaccagct

SEQ ID NO: 109 - DNA, 51 bp, Homo sapiens
  allele (26)...(0): single nucleotide polymorphism
acccgttcttctgcccacccactgaagccccagaccgtgacttcttggtgg

SEQ ID NO: 110 - DNA, 51 bp, Homo sapiens
  allele (26)...(0): single nucleotide polymorphism
tctggaagccggacatcctctgagcgagtcgactgatccgctggcgaacca

SEQ ID NO: 111 - DNA, 51 bp, Homo sapiens
  allele (26)...(0): single nucleotide polymorphism
tcatcagagattcgatctcctcgtcagtcacgtgctccccggaggccctga

SEQ ID NO: 112 - DNA, 51 bp, Homo sapiens
  allele (26)...(0): single nucleotide polymorphism
gctttgaggaggaggcgcggttgcgggacgacactgaggcggccatccgcg

SEQ ID NO: 113 - DNA, 51 bp, Homo sapiens
  allele (26)...(0): single nucleotide polymorphism
ttcggaaagggcaagcagtgaccctcatgatggatgccaccaatatgccag

SEQ ID NO: 114 - DNA, 51 bp, Homo sapiens
  allele (26)...(0): single nucleotide polymorphism
acacccacagcagttttggtttaggtttatctgtaaatggaaggttctggc

SEQ ID NO: 115 - DNA, 51 bp, Homo sapiens
  allele (26)...(0): single nucleotide polymorphism
tcttctccaacagtctgccacccgctgtcgttggctgcgcctccaaggccc

SEQ ID NO: 116 - DNA, 51 bp, Homo sapiens
  allele (26)...(0): single nucleotide polymorphism
ttgtggtcctggcctcgcagttcctatcccatgacccagaacagctcacca

SEQ ID NO: 117 - DNA, 51 bp, Homo sapiens
  allele (26)...(0): single nucleotide polymorphism
tcacagggaaaattcaacgagccaaacttcgagacaaggagtggaagatgt

SEQ ID NO: 118 - DNA, 51 bp, Homo sapiens
  allele (26)...(0): single nucleotide polymorphism
tgactctccccgacccgtcccaccatggtctccacagcactcccgacagcc

SEQ ID NO: 119 - DNA, 51 bp, Homo sapiens
  allele (26)...(0): single nucleotide polymorphism
tccgcaaggccttcctgaagatccttcactgctgactctgctgcctgcccg

SEQ ID NO: 120 - DNA, 51 bp, Homo sapiens
  allele (26)...(0): single nucleotide polymorphism
tcctcgtcgccacactggtcatgccatgggttgtctacctggaggtggtag

SEQ ID NO: 121 - DNA, 51 bp, Homo sapiens
  allele (26)...(0): single nucleotide polymorphism
ttgctctttgctggttccctcttcatttaagccgtatattgaagaaaactg

SEQ ID NO: 122 - DNA, 51 bp, Homo sapiens
  allele (26)...(0): single nucleotide polymorphism
tattgaagaaaactgtgtataacgagatggacaagaaccgatgtgaattac

SEQ ID NO: 123 - DNA, 51 bp, Homo sapiens
  allele (26)...(0): single nucleotide polymorphism
acgtgaacaccgacatctactccaaagtgctggtgaccgccgtgtacctgg

SEQ ID NO: 124 - DNA, 51 bp, Homo sapiens
  allele (26)...(0): single nucleotide polymorphism
attccttgattgctaggaccctttataaaagcaccctgaacatacctactg

SEQ ID NO: 125 - DNA, 51 bp, Homo sapiens
  allele (26)...(0): single nucleotide polymorphism
cttatgctgtgatcatttcagtgggtatccttggaaatgctattctcatca

SEQ ID NO: 126 - DNA, 51 bp, Homo sapiens
  allele (26)...(0): single nucleotide polymorphism
tccgaaagaagtcttgggaggtgtatcagggagtgtgccagaaagggggct

SEQ ID NO: 127 - DNA, 51 bp, Homo sapiens
  allele (26)...(0): single nucleotide polymorphism
agacacccctttcccagctcgcctcagggaggagggacccaagggccccct

SEQ ID NO: 128 - DNA, 51 bp, Homo sapiens
  allele (26)...(0): single nucleotide polymorphism
gcccctcggccttcgcggtgctggtgaccggactggcggccaccgacctgc

SEQ ID NO: 129 - DNA, 51 bp, Homo sapiens
  allele (26)...(0): single nucleotide polymorphism
ccagactggtcctggtggtggtggcggtcttcgtcgtctgctggactccca

SEQ ID NO: 130 - DNA, 51 bp, Homo sapiens
  allele (26)...(0): single nucleotide polymorphism
cagcactcaccatggaatccccgattcagatcttccgcggggagccgggcc

SEQ ID NO: 131 - DNA, 51 bp, Homo sapiens
  allele (26)...(0): single nucleotide polymorphism
cgatccagatcttccgcggggagcctggccctacctgcgccccgagcgcct

SEQ ID NO: 132 - DNA, 51 bp, Homo sapiens
  allele (26)...(0): single nucleotide polymorphism
tggatctgcacctcttcgactactccgagccagggaacttctcggacatca

SEQ ID NO: 133 - DNA, 51 bp, Homo sapiens
  allele (26)...(0): single nucleotide polymorphism
cgctgcacctgtgcatcgcgctgggttacgccaatagcagcctcaaccccg

SEQ ID NO: 134 - DNA, 51 bp, Homo sapiens
  allele (26)...(0): single nucleotide polymorphism
tggctgtgacccgtccccgggacggtgcagtggtgtgcatgctccagttcc

SEQ ID NO: 135 - DNA, 51 bp, Homo sapiens
  allele (26)...(0): single nucleotide polymorphism
tggccttcccgatcaccatgctgctgactggtttcgtgggcaacgcactgg

SEQ ID NO: 136 - DNA, 51 bp, Homo sapiens
  allele (26)...(0): single nucleotide polymorphism
gggatgccaccttctgcttcatcgtgtcgctggcggtggctgatgtggccg

SEQ ID NO: 137 - DNA, 51 bp, Homo sapiens
  allele (26)...(0): single nucleotide polymorphism
ccatctccttctgtggctgtctcacgcagatgtatttcgttttcatgttcg

SEQ ID NO: 138 - DNA, 51 bp, Homo sapiens
  allele (26)...(0): single nucleotide polymorphism
ggtggaaagccttctccacctgtggctctcacctggctgtggttctcctct

SEQ ID NO: 139 - DNA, 51 bp, Homo sapiens
  allele (26)...(0): single nucleotide polymorphism
acagcaccatcattgctgtgtatttcaaccctctgtcctcccactcagctg

SEQ ID NO: 140 - DNA, 51 bp, Homo sapiens
  allele (26)...(0): single nucleotide polymorphism
actctccaatgtactttttcctctctaacctctccttcttggacctctgct

SEQ ID NO: 141 - DNA, 51 bp, Homo sapiens
  allele (26)...(0): single nucleotide polymorphism
gacatcaggcgcacggtgccaacctgcgccatctgcaggccaagaagaagt

SEQ ID NO: 142 - DNA, 51 bp, Homo sapiens
  allele (26)...(0): single nucleotide polymorphism
aggcgcacggtgccaacctccgccacctgcaggccaagaagaagtttgtga

SEQ ID NO: 143 - DNA, 51 bp, Homo sapiens
  allele (26)...(0): single nucleotide polymorphism
cagccttctccatgcccagctggcaactggcactgtgggcaccagcctacc

SEQ ID NO: 144 - DNA, 51 bp, Homo sapiens
  allele (26)...(0): single nucleotide polymorphism
cctgtgctgatctggtcatgggcctagcagtggtgccctttggggccgccc

SEQ ID NO: 145 - DNA, 51 bp, Homo sapiens
  allele (26)...(0): single nucleotide polymorphism
cttgcccattcagatgcactggtacagggccacccaccaggaagccatcaa

SEQ ID NO: 146 - DNA, 51 bp, Homo sapiens
  allele (26)...(0): single nucleotide polymorphism
ttggaagcgtgcatccagtgagacctatgaggcttgagtcttttagtgcct

SEQ ID NO: 147 - DNA, 51 bp, Homo sapiens
  allele (26)...(0): single nucleotide polymorphism
gtgtgagcagagatgccagaaccaaggtggaccgaacaccattacatatgg

SEQ ID NO: 148 - DNA, 51 bp, Homo sapiens
  allele (26)...(0): single nucleotide polymorphism
tggcagctaccagcacactgcctccgccgtcaataaaggcactgatggtct

SEQ ID NO: 149 - DNA, 51 bp, Homo sapiens
  allele (26)...(0): single nucleotide polymorphism
tggctcccattgtctgggagggcacgttcaacatcgacatcctcaacgagc

SEQ ID NO: 150 - DNA, 51 bp, Homo sapiens
  allele (26)...(0): single nucleotide polymorphism
acgtggacatggagttccgcgaccatgtgggcgtggagatcctgactccgc

SEQ ID NO: 151 - DNA, 51 bp, Homo sapiens
  allele (26)...(0): single nucleotide polymorphism
acgtgggcgtggagatcctgactccactgttcggcaccctgcaccccggct

SEQ ID NO: 152 - DNA, 51 bp, Homo sapiens
  allele (26)...(0): single nucleotide polymorphism
ggccccagtcccaggcctacatccctaaggacgagggcgatttctactacc

SEQ ID NO: 153 - DNA, 51 bp, Homo sapiens
  allele (26)...(0): single nucleotide polymorphism
acgagagccacctgaacaagtacctactgcgccacaaacccaccaaggtgc

SEQ ID NO: 154 - DNA, 51 bp, Homo sapiens
  allele (26)...(0): single nucleotide polymorphism
ggggagatactggctcacccaggaacacagggaacatcaccttatgccaca

SEQ ID NO: 155 - DNA, 51 bp, Homo sapiens
  allele (26)...(0): single nucleotide polymorphism
acgcagtggccgtgggctccctctgtgctctttccgccagtcttctaggtt

SEQ ID NO: 156 - DNA, 51 bp, Homo sapiens
  allele (26)...(0): single nucleotide polymorphism
ccatcgccacgctccctctgtcctcggcctgggccgtggtcttcttcatca

SEQ ID NO: 157 - DNA, 51 bp, Homo sapiens
  allele (26)...(0): single nucleotide polymorphism
gatggaacagctcctcgggtgtcttatcactttggctggctcccccctgcc

SEQ ID NO: 158 - DNA, 51 bp, Homo sapiens
  allele (26)...(0): single nucleotide polymorphism
gcggctgctggtggatgggtgggcggggggtgcagcctccaccccctcccc

SEQ ID NO: 159 - DNA, 51 bp, Homo sapiens
  allele (26)...(0): single nucleotide polymorphism
cgcctgtaatggctgtgaacatgcttacccagcaggaggtccctgtcgtta

SEQ ID NO: 160 - DNA, 51 bp, Homo sapiens
  allele (26)...(0): single nucleotide polymorphism
cattgactaggggctgtgggggcatgcgcccaggtgtccctccatcagagg

SEQ ID NO: 161 - DNA, 51 bp, Homo sapiens
  allele (26)...(0): single nucleotide polymorphism
agcaggccaagagagatctgtggaatgcatcttgttccagaataccagata

SEQ ID NO: 162 - DNA, 51 bp, Homo sapiens
  allele (26)...(0): single nucleotide polymorphism
cgctggcataggacatggcgggctttccccccgcagagctctgggggctac

SEQ ID NO: 163 - DNA, 51 bp, Homo sapiens
  allele (26)...(0): single nucleotide polymorphism
aaataacaaggcattgaagaatggcagacgagcggaaagacgaaggaaagg

SEQ ID NO: 164 - DNA, 51 bp, Homo sapiens
  allele (26)...(0): single nucleotide polymorphism
aaggacgcaacgctgccaccatggatagtagcacctggagccccaagacca

SEQ ID NO: 165 - DNA, 51 bp, Homo sapiens
  allele (26)...(0): single nucleotide polymorphism
tgaaagtattcaatcccagaaggaagctggaatttgcccttctgtttctag

SEQ ID NO: 166 - DNA, 51 bp, Homo sapiens
  allele (26)...(0): single nucleotide polymorphism
gctggcgcactgctagcctcagaggagccagcacctcctcagcccccgcgc

SEQ ID NO: 167 - DNA, 51 bp, Homo sapiens
  allele (26)...(0): single nucleotide polymorphism
aaaaccagctacctgcctttctggaggaactttgccatgagaaagaaattt

SEQ ID NO: 168 - DNA, 51 bp, Homo sapiens
  allele (26)...(0): single nucleotide polymorphism
catgagttttgatcccagctcttctttccctggctttctgggccatttctc

SEQ ID NO: 169 - DNA, 51 bp, Homo sapiens
  allele (26)...(0): single nucleotide polymorphism
ttggagctggaattactgtgtatgaggccttagcagctgctgatgagcttt

SEQ ID NO: 170 - DNA, 51 bp, Homo sapiens
  allele (26)...(0): single nucleotide polymorphism
tcaacacctacgtccacttccaaggtaagatgaagggcttctccctgctgg

SEQ ID NO: 171 - DNA, 51 bp, Homo sapiens
  allele (26)...(0): single nucleotide polymorphism
tggcttgcacaaattgcttgaagactcgatccatgtaagtggactgtcttg

SEQ ID NO: 172 - DNA, 51 bp, Homo sapiens
  allele (26)...(0): single nucleotide polymorphism
ctgggcagctgccctcacagtagttgccgtagtagccggtgggtgctatga

SEQ ID NO: 173 - DNA, 51 bp, Homo sapiens
  allele (26)...(0): single nucleotide polymorphism
gccgagcctgcaccaccacaaagggtcggtgcgactcttcgcctgggtcca

SEQ ID NO: 174 - DNA, 51 bp, Homo sapiens
  allele (26)...(0): single nucleotide polymorphism
catagaaggccaggagtcaggagacttgggttctgtcctggattatacacc

SEQ ID NO: 175 - DNA, 51 bp, Homo sapiens
  allele (26)...(0): single nucleotide polymorphism
ggagtcaggagacctgggttctgtcttggattatacaccagctcactgagg

SEQ ID NO: 176 - DNA, 51 bp, Homo sapiens
  allele (26)...(0): single nucleotide polymorphism
ccatgcccacccccgacgccaccaccccacaggccaagggcttccgcaggg

SEQ ID NO: 177 - DNA, 51 bp, Homo sapiens
  allele (26)...(0): single nucleotide polymorphism
atttaatgaatttcctgaagactgtgagaagtacaactgagaaatcccttt

SEQ ID NO: 178 - DNA, 51 bp, Homo sapiens
  allele (26)...(0): single nucleotide polymorphism
aaaacaatgatatcgatgaagttattattcccacagctcccttatacaaac

SEQ ID NO: 179 - DNA, 51 bp, Homo sapiens
  allele (26)...(0): single nucleotide polymorphism
caccatgaagcagttgctgcgggccttggaggagggccgcgtgcgggaagt

SEQ ID NO: 180 - DNA, 51 bp, Homo sapiens
  allele (26)...(0): single nucleotide polymorphism
tcctgtacaaagacaggaacctccatattcccaccatggaaaatgggcctg

SEQ ID NO: 181 - DNA, 51 bp, Homo sapiens
  allele (26)...(0): single nucleotide polymorphism
ctgcggtggagacgtcagagctgccgggggagggggctcctgcgccacagc

SEQ ID NO: 182 - DNA, 51 bp, Homo sapiens
  allele (26)...(0): single nucleotide polymorphism
accagctgctcgtagtacacaggcaagcacttctccttgcctacctccatg

SEQ ID NO: 183 - DNA, 51 bp, Homo sapiens
  allele (26)...(0): single nucleotide polymorphism
ctaccgccaactatgactttgtcctgaagaagcggaccttcaccaagggag

SEQ ID NO: 184 - DNA, 51 bp, Homo sapiens
  allele (26)...(0): single nucleotide polymorphism
tcccctggcagaactaccacctgaatgactggatggaggaggaataccgcc

SEQ ID NO: 185 - DNA, 51 bp, Homo sapiens
  allele (26)...(0): single nucleotide polymorphism
acatccaggtggtgttcgacgccgttaccgacatcatcattgccaacaacc

SEQ ID NO: 186 - DNA, 51 bp, Homo sapiens
  allele (26)...(0): single nucleotide polymorphism
cagtgacggcagggtcaaagtccttagcgtagccctcgttaaggctgtaga

SEQ ID NO: 187 - DNA, 51 bp, Homo sapiens
  allele (26)...(0): single nucleotide polymorphism
aaccagcccactgtgagaagaccaccgtgttcaagtctttgggaatggcag

SEQ ID NO: 188 - DNA, 51 bp, Homo sapiens
  allele (26)...(0): single nucleotide polymorphism
ccacaatgttaggagggtatttttatatccctccagttaacaaatacagca

SEQ ID NO: 189 - DNA, 51 bp, Homo sapiens
  allele (26)...(0): single nucleotide polymorphism
ctgccatctttcagccctctgaaactgtgtccagcacagaatcttccctgg

SEQ ID NO: 190 - DNA, 51 bp, Homo sapiens
  allele (26)...(0): single nucleotide polymorphism
gcgcttcccaggtccggacaattcgtcagactattgtcaaactggggaata

SEQ ID NO: 191 - DNA, 51 bp, Homo sapiens
  allele (26)...(0): single nucleotide polymorphism
ggcagctgaagatcaccaatgctggcatggtgtctgatgaggagttggagc

SEQ ID NO: 192 - DNA, 51 bp, Homo sapiens
  allele (26)...(0): single nucleotide polymorphism
gcgaggtgtttgtgtccaatatccttaaggacacgcaggtgactcgacagg

SEQ ID NO: 193 - DNA, 51 bp, Homo sapiens
  allele (26)...(0): single nucleotide polymorphism
tgtacactgccagaaaaggaaaaggggccttttgtaatggtcaaaaactac

SEQ ID NO: 194 - DNA, 51 bp, Homo sapiens
  allele (26)...(0): single nucleotide polymorphism
tcttggtgactgagttgggctcttctagaacaccagagactgtgagaatgg

SEQ ID NO: 195 - DNA, 51 bp, Homo sapiens
  allele (26)...(0): single nucleotide polymorphism
tagagcgcacacaggcctccagctgggccatgtccgtctcatcatcccaag

SEQ ID NO: 196 - DNA, 51 bp, Homo sapiens
  allele (26)...(0): single nucleotide polymorphism
ctgacagctacaggctctttcagtttcattttcactggggcagtacaaatg

SEQ ID NO: 197 - DNA, 51 bp, Homo sapiens
  allele (26)...(0): single nucleotide polymorphism
agaagttgaaggggctggtgccactgggacccgaatcaagtcgacacacta

SEQ ID NO: 198 - DNA, 51 bp, Homo sapiens
  allele (26)...(0): single nucleotide polymorphism
tgggttcagggatgtagcccttctccacagccaggcggctcagggcaaaca

SEQ ID NO: 199 - DNA, 51 bp, Homo sapiens
  allele (26)...(0): single nucleotide polymorphism
gccaatataggatagggcactacaggttccggtacagtgacaccctggagc

SEQ ID NO: 200 - DNA, 51 bp, Homo sapiens
  allele (26)...(0): single nucleotide polymorphism
acggggaggagctgcagatggaacctgtgtgaggtgtcttctgggacctgc

SEQ ID NO: 201 - DNA, 51 bp, Homo sapiens
  allele (26)...(0): single nucleotide polymorphism
agaggttggggggcgccgagcgcgaacggccccgaaaggggctgggctcct

SEQ ID NO: 202 - DNA, 51 bp, Homo sapiens
  allele (26)...(0): single nucleotide polymorphism
tagtgaaaggcctgaaatatatgctcgaggtggaaattggcagaactacct

SEQ ID NO: 203 - DNA, 51 bp, Homo sapiens
  allele (26)...(0): single nucleotide polymorphism
ctccatcaacagcatccggactgcacggcggctcgccgtgcggctggggcc

SEQ ID NO: 204 - DNA, 51 bp, Homo sapiens
  allele (26)...(0): single nucleotide polymorphism
cgacgaggtgctacgcgagggcgagttggagaagcgcagcgacagcctctt

SEQ ID NO: 205 - DNA, 51 bp, Homo sapiens
  allele (26)...(0): single nucleotide polymorphism
ttttttccagcttacaatggtacaggcaggagcctggggaaggtcctgtcc

SEQ ID NO: 206 - DNA, 51 bp, Homo sapiens
  allele (26)...(0): single nucleotide polymorphism
agaagagagcctgtgacactgccacttgtgtgactcatcggctggcaggct

SEQ ID NO: 207 - DNA, 51 bp, Homo sapiens
  allele (26)...(0): single nucleotide polymorphism
tcatcctgagttctaagaagctcctcctcagtgactctggcttctatctct

SEQ ID NO: 208 - DNA, 51 bp, Homo sapiens
  allele (26)...(0): single nucleotide polymorphism
ctctggttgtccacgagggagacaccgtaactctcaattgcagttatgaag

SEQ ID NO: 209 - DNA, 51 bp, Homo sapiens
  allele (26)...(0): single nucleotide polymorphism
aagtctgtgctgatccacaagccacgtgggtgagagacgtggtcaggagca

SEQ ID NO: 210 - DNA, 50 bp, Homo sapiens
  allele (26)...(0): single nucleotide polymorphism
agggaggcggggagggtagcatgggcacacggccctcacagggactcact

SEQ ID NO: 211 - DNA, 51 bp, Homo sapiens
  allele (26)...(0): single nucleotide polymorphism
agttgaaatcagagaggaataaaaaagacattttatattttattctgctcc

SEQ ID NO: 212 - DNA, 51 bp, Homo sapiens
  allele (26)...(0): single nucleotide polymorphism
taagcatgaggtggcacgaggcaggcgttggcgatgccacctgggggtcac

SEQ ID NO: 213 - DNA, 51 bp, Homo sapiens
  allele (26)...(0): single nucleotide polymorphism
ggtccccttgctttatcccaagctctgagggacgcagcctggcatggctct

SEQ ID NO: 214 - DNA, 51 bp, Homo sapiens
  allele (26)...(0): single nucleotide polymorphism
gtccccttgctttatcccaagctcgtagggacgcagcctggcatggctctg

SEQ ID NO: 215 - DNA, 51 bp, Homo sapiens
  allele (26)...(0): single nucleotide polymorphism
ctttatcccaagctcggagggacgcgagcctggcatggctctggcctagca

SEQ ID NO: 216 - DNA, 51 bp, Homo sapiens
  allele (26)...(0): single nucleotide polymorphism
tagcagccaggtgacatggccaggctaccttcctgtacaggcactgtgggc

SEQ ID NO: 217 - DNA, 51 bp, Homo sapiens
  allele (26)...(0): single nucleotide polymorphism
gccaggtgacatggccaggcacctttcctgtacaggcactgtgggctcctg

SEQ ID NO: 218 - DNA, 51 bp, Homo sapiens
  allele (26)...(0): single nucleotide polymorphism
aggtgacatggccaggcaccttccttgtacaggcactgtgggctcctggcc

SEQ ID NO: 219 - DNA, 51 bp, Homo sapiens; identical to SEQ ID NO: 218
  allele (26)...(0): single nucleotide polymorphism

SEQ ID NO: 220 - DNA, 51 bp, Homo sapiens; identical to SEQ ID NO: 218
  allele (26)...(0): single nucleotide polymorphism

SEQ ID NO: 221 - DNA, 51 bp, Homo sapiens
  allele (26)...(0): single nucleotide polymorphism
aatccacaatcggcatcaggaagcccaagtcccagtggccattagggtcct

SEQ ID NO: 222 - DNA, 51 bp, Homo sapiens
  allele (26)...(0): single nucleotide polymorphism
tccctatgagcctgcaaaggagacattcaggaatgagttccatgttcgaga

SEQ ID NO: 223 - DNA, 51 bp, Homo sapiens
  allele (26)...(0): single nucleotide polymorphism
cagtgcatctgggaagatttctacccgaccaacagttccttcagcttccat

SEQ ID NO: 224 - DNA, 51 bp, Homo sapiens
  allele (26)...(0): single nucleotide polymorphism
caacagttccttcagcttccatttcacccctcatttatccctcaaccccca

SEQ ID NO: 225 - DNA, 51 bp, Homo sapiens
  allele (26)...(0): single nucleotide polymorphism
tgctctcctttcccctgcccccagaacttttatccacttacctagattcta

SEQ ID NO: 226 - DNA, 51 bp, Homo sapiens
  allele (26)...(0): single nucleotide polymorphism
tggccacagtgaaaaaggtcatgggaggagagaagcaaagtaggaaggatc

SEQ ID NO: 227 - DNA, 51 bp, Homo sapiens
  allele (26)...(0): single nucleotide polymorphism
accgcaccctttccaccggtggggggcccagtgaagtttaacaaactgctg

SEQ ID NO: 228 - DNA, 51 bp, Homo sapiens
  allele (26)...(0): single nucleotide polymorphism
cataccacgttcactgcaagggggggaacgtgtgggttgctctattcaaga

SEQ ID NO: 229 - DNA, 51 bp, Homo sapiens
  allele (26)...(0): single nucleotide polymorphism
gaaacccagtaggctcctggaggccctggtcagcttgcttggaatccagca

SEQ ID NO: 230 - DNA, 51 bp, Homo sapiens
  allele (26)...(0): single nucleotide polymorphism
tggtggtgctacccttggcctcccagagtcctgccaccctgctgccgccac

SEQ ID NO: 231 - DNA, 51 bp, Homo sapiens
  allele (26)...(0): single nucleotide polymorphism
agcccttctccacccggatagattcttcacccttggcccgcctttgcccca

SEQ ID NO: 232 - DNA, 51 bp, Homo sapiens
  allele (26)...(0): single nucleotide polymorphism
acccggatagattcctcacccttggtccgcctttgccccaccctactctgc

SEQ ID NO: 233 - DNA, 51 bp, Homo sapiens
  allele (26)...(0): single nucleotide polymorphism
gtgcctggacatttgccttgctggatggggactggggatgtgggagggagc

SEQ ID NO: 234 - DNA, 51 bp, Homo sapiens
  allele (26)...(0): single nucleotide polymorphism
acgactttgagcctcgcgatcttttgagtccaacgtccagctcgttctctg

SEQ ID NO: 235 - DNA, 51 bp, Homo sapiens
  allele (26)...(0): single nucleotide polymorphism
tgcggcttaaaagggcaacccgcgccggacccttcctccctagtcgcgggg

SEQ ID NO: 236 - DNA, 51 bp, Homo sapiens
  allele (26)...(0): single nucleotide polymorphism
gagagaccatttacttacatcagtttggtttatagacatttgaatcatatc

SEQ ID NO: 237 - DNA, 51 bp, Homo sapiens
  allele (26)...(0): single nucleotide polymorphism
agtttcattatacttttctctccacgttttgtctatgttgaaaattttctg

SEQ ID NO: 238 - DNA, 51 bp, Homo sapiens
  allele (26)...(0): single nucleotide polymorphism
acaagacagaagctgaagtgcatcccaaaggtgctcagagagccggcccgc

SEQ ID NO: 239 - DNA, 51 bp, Homo sapiens
  allele (26)...(0): single nucleotide polymorphism
tcttccttctccagccggcaggcccgcgccgcttaggagggagagcccacc

SEQ ID NO: 240 - DNA, 51 bp, Homo sapiens
  allele (26)...(0): single nucleotide polymorphism
tggcgagtccagggtcacccacataaccatgcaccacgggtgctatgccgc

SEQ ID NO: 241 - DNA, 51 bp, Homo sapiens
  allele (26)...(0): single nucleotide polymorphism
gagtccagggtcacccacataccattgcaccacgggtgctatgccgcttct

SEQ ID NO: 242 - DNA, 51 bp, Homo sapiens
  allele (26)...(0): single nucleotide polymorphism
taccatgcaccacgggtgctatgccacttcttacaggacctttttagccct

SEQ ID NO: 243 - DNA, 51 bp, Homo sapiens
  allele (26)...(0): single nucleotide polymorphism
cctggaggcaactgggtagggtgcacaacggcatgctttggctggaacacg

SEQ ID NO: 244 - DNA, 51 bp, Homo sapiens
  allele (26)...(0): single nucleotide polymorphism
cagaacggcatgctttggctggaaccacgcatccctccttccacggccggc

SEQ ID NO: 245 - DNA, 51 bp, Homo sapiens
  allele (26)...(0): single nucleotide polymorphism
cagagctagctctggctcttcaggctacaagttcacagtccttcgctcctg

SEQ ID NO: 246 - DNA, 51 bp, Homo sapiens
  allele (26)...(0): single nucleotide polymorphism
gtccttcgctcctgagcaccaggttcagtctccaggaagggatttggtgaa

SEQ ID NO: 247 - DNA, 51 bp, Homo sapiens
  allele (26)...(0): single nucleotide polymorphism
gatacctttgcgtggatcaagcttgctgtacttgaccgtttttatattact

SEQ ID NO: 248 - DNA, 51 bp, Homo sapiens
  allele (26)...(0): single nucleotide polymorphism
caaaaaaatttataaactaattttggtacgtatgaatgatatctttgacct

SEQ ID NO: 249 - DNA, 51 bp, Homo sapiens
  allele (26)...(0): single nucleotide polymorphism
cctccttctccccctttcccctccccgcccccaccttcttcctcctttcgg

SEQ ID NO: 250 - DNA, 51 bp, Homo sapiens
  allele (26)...(0): single nucleotide polymorphism
ctgccctgccacctgtctgtctgtctccaaagaagttctggtatgaacttg

SEQ ID NO: 251 - DNA, 51 bp, Homo sapiens; identical to SEQ ID NO: 250
  allele (26)...(0): single nucleotide polymorphism

SEQ ID NO: 252 - DNA, 51 bp, Homo sapiens
  allele (26)...(0): single nucleotide polymorphism
tccctccaggactaggctggaggaacccagtggggtcccccctgagtgggc

SEQ ID NO: 253 - DNA, 51 bp, Homo sapiens
  allele (26)...(0): single nucleotide polymorphism
cacatgtggggacagggctggtgtgcctgctcccagcctcttgctcagagc

SEQ ID NO: 254 - DNA, 51 bp, Homo sapiens
  allele (26)...(0): single nucleotide polymorphism
ctaaagtcggagtatcttcttccaaaatttcacgtcttggcggccgttcca

SEQ ID NO: 255 - DNA, 51 bp, Homo sapiens
  allele (26)...(0): single nucleotide polymorphism
tttctagagggggtctgttgaagatatgtaactagtacaccccaaccccca

SEQ ID NO: 256 - DNA, 51 bp, Homo sapiens
  allele (26)...(0): single nucleotide polymorphism
ccccaacccccaacctcagtggaaagcaatgcccagggattaggctatgga

SEQ ID NO: 257 - DNA, 51 bp, Homo sapiens
  allele (26)...(0): single nucleotide polymorphism
gcgcaggtcagagggcggccgcagcgggcctccgcgaggtccccacgccgg

SEQ ID NO: 258 - DNA, 50 bp, Homo sapiens
  allele (26)...(0): single nucleotide polymorphism
ctctatactgtacactcacccataatcaaacaattacaccatggtataaa

SEQ ID NO: 259 - DNA, 50 bp, Homo sapiens
  allele (26)...(0): single nucleotide polymorphism
tctatactgtacactcacccataatcaaacaattacaccatggtataaag

SEQ ID NO: 260 - DNA, 51 bp, Homo sapiens
  allele (26)...(0): single nucleotide polymorphism
gccgaatagcctgggtttggaaaagtatgtttttgaaatatgtgggatctc

SEQ ID NO: 261 - DNA, 51 bp, Homo sapiens
  allele (26)...(0): single nucleotide polymorphism
tactgacctaaatcacaccctagacttatcagagggaaattctgaccataa

SEQ ID NO: 262 - DNA, 51 bp, Homo sapiens
  allele (26)...(0): single nucleotide polymorphism
tgtccttgaagaacatgcacttggcgcggatggcacaagcaaaatggtaga

SEQ ID NO: 263 - DNA, 51 bp, Homo sapiens
  allele (26)...(0): single nucleotide polymorphism
cccgcgccccagtaggagccccgcggcccagcaggtgcggcgcgcacggag

SEQ ID NO: 264 - DNA, 51 bp, Homo sapiens
  allele (26)...(0): single nucleotide polymorphism
ccagcaggtgcggcgcgcacggagcgcgccggccggcggcttctcccggag

SEQ ID NO: 265 - DNA, 51 bp, Homo sapiens
  allele (26)...(0): single nucleotide polymorphism
tgaaacttgaaaccgcctctggagctgccattctgcagagtatttggaaaa

SEQ ID NO: 266 - DNA, 51 bp, Homo sapiens
  allele (26)...(0): single nucleotide polymorphism
tccaagaaagggtcatggaagcttactgggaataatcctctcaattagaaa

SEQ ID NO: 267 - DNA, 51 bp, Homo sapiens
  allele (26)...(0): single nucleotide polymorphism
ggggggttttttttttttttctctgtttttttttttttttttttttttttt

SEQ ID NO: 268 - DNA, 50 bp, Homo sapiens
  allele (26)...(0): single nucleotide polymorphism
ctgggggttttcggggaggaaccaaggctcacggagcctcctgtgctgca

SEQ ID NO: 269 - DNA, 50 bp, Homo sapiens
  allele (26)...(0): single nucleotide polymorphism
gggggttttcggggaggaaccaaggctcacggagcctcctgtgctgcagt

SEQ ID NO: 270 - DNA, 51 bp, Homo sapiens
  allele (26)...(0): single nucleotide polymorphism
gagttaatttatgtaagtcatattttatatttttaagaagtaccacttgaa

SEQ ID NO: 271 - DNA, 51 bp, Homo sapiens
  allele (26)...(0): single nucleotide polymorphism
cttacctcaaataaatggctaactttatacatatttttaaagaaatattta

SEQ ID NO: 272 - DNA, 51 bp, Homo sapiens
  allele (26)...(0): single nucleotide polymorphism
cagcccccattgtggtcacaggaagcagaggaggccacgttcttactagtt

SEQ ID NO: 273 - DNA, 51 bp, Homo sapiens
  allele (26)...(0): single nucleotide polymorphism
cccaacctgggtttggcagacatcagaatgatggagtacattttgcagata

SEQ ID NO: 274 - DNA, 51 bp, Homo sapiens
  allele (26)...(0): single nucleotide polymorphism
cacccccaggttctcctagttcagaaaaaagctgtgaaagtggaagaagga

SEQ ID NO: 275 - DNA, 51 bp, Homo sapiens
  allele (26)...(0): single nucleotide polymorphism
tttattctattcctatctgtggatgggtaaatggctggggggccagccctg

SEQ ID NO: 276 - DNA, 51 bp, Homo sapiens
  allele (26)...(0): single nucleotide polymorphism
agcctttgtgctcccactcaatacacaaaggcccctctctacatctgggaa

SEQ ID NO: 277 - DNA, 51 bp, Homo sapiens
  allele (26)...(0): single nucleotide polymorphism
aaaaaggcccctctctacatctggggatgcacctcttctttgattccctgg

SEQ ID NO: 278 - DNA, 51 bp, Homo sapiens
  allele (26)...(0): single nucleotide polymorphism
agcaacttggctgagccccactacatacagagaaatcatcaacctgactta

SEQ ID NO: 279 - DNA, 51 bp, Homo sapiens
  allele (26)...(0): single nucleotide polymorphism
taagagttttcaagatgtcaaacttaaggctgatcagcagatgggatgtga

SEQ ID NO: 280 - DNA, 51 bp, Homo sapiens
  allele (26)...(0): single nucleotide polymorphism
tttttaaaaatccatccacacacattggtaaattaagtataaattcttttg

SEQ ID NO: 281 - DNA, 50 bp, Homo sapiens
  allele (26)...(0): single nucleotide polymorphism
aacgtcgattcgcaccgtccaacctgccccgcccctcctacagctgtaac

SEQ ID NO: 282 - DNA, 50 bp, Homo sapiens
  allele (26)...(0): single nucleotide polymorphism
acgtcgattcgcaccgtccaacctgccccgcccctcctacagctgtaact

SEQ ID NO: 283 - DNA, 51 bp, Homo sapiens
  allele (26)...(0): single nucleotide polymorphism
cacttaataccagagaccccccccccttcccctcccccttcccctccccct

SEQ ID NO: 284 - DNA, 51 bp, Homo sapiens
  allele (26)...(0): single nucleotide polymorphism
agacgtgtctgccacaggtctcagggtaacagatgccctgtccactgagag

SEQ ID NO: 285 - DNA, 51 bp, Homo sapiens
  allele (26)...(0): single nucleotide polymorphism
tttgatggaaaggttgtccacactgagaattatcacacacttgatcaggaa

SEQ ID NO: 286 - DNA, 51 bp, Homo sapiens
  allele (26)...(0): single nucleotide polymorphism
ccctccggattcggcgcgcgtgcggmccgccgcgagtgagggttttcgtgg

SEQ ID NO: 287 - DNA, 51 bp, Homo sapiens
  allele (26)...(0): single nucleotide polymorphism
tccaagctaagcactgccactgggggaaactccaccttcccactttcccac

SEQ ID NO: 288 - DNA, 51 bp, Homo sapiens
  allele (26)...(0): single nucleotide polymorphism
ccacctccatcccagacaggtccctgcccttctctgtgcagtagcatcacc

SEQ ID NO: 289 - DNA, 51 bp, Homo sapiens
  allele (26)...(0): single nucleotide polymorphism
cacctccatcccagacaggtccctcgccttctctgtgcagtagcatcacct

SEQ ID NO: 290 - DNA, 51 bp, Homo sapiens
  allele (26)...(0): single nucleotide polymorphism
gtctgatagaagaggagcaggagaagcaaatcgttaaaacctagcgaattc

SEQ ID NO: 291 - DNA, 51 bp, Homo sapiens
  allele (26)...(0): single nucleotide polymorphism
tcaggagcaaggcgaatgtatgacaccatgtccacaatggtgtacataaag

SEQ ID NO: 292 - DNA, 51 bp, Homo sapiens
  allele (26)...(0): single nucleotide polymorphism
ttctgaagaggctgacgattttactgtctcatttttttcctttctccagaa

SEQ ID NO: 293 - DNA, 51 bp, Homo sapiens
  allele (26)...(0): single nucleotide polymorphism
ctagcttcccttcccattcaacacacacacacattcttgctctacccaaag

SEQ ID NO: 294 - DNA, 51 bp, Homo sapiens
  allele (26)...(0): single nucleotide polymorphism
tgtctcaaacccagcttgccagctccaatgtaccagcagctggaatctgaa

SEQ ID NO: 295 - DNA, 51 bp, Homo sapiens
  allele (26)...(0): single nucleotide polymorphism
gtggctgggctattccatccatctggaagcacatttgagcctccaggcttc

SEQ ID NO: 296 - DNA, 51 bp, Homo sapiens
  allele (26)...(0): single nucleotide polymorphism
cgagcggcacccagagcctgcacccgccctcaccgtccttctgcgtccccc

SEQ ID NO: 297 - DNA, 51 bp, Homo sapiens
  allele (26)...(0): single nucleotide polymorphism
aggagccctcttcggtgtccccgagcgccacggtcaagacccgcagcacca

SEQ ID NO: 298 - DNA, 51 bp, Homo sapiens
  allele (26)...(0): single nucleotide polymorphism
cggtcaagacccgcagcaccaaagcgccgcccccgcacctgcccctgtcgc

SEQ ID NO: 299 - DNA, 51 bp, Homo sapiens
  allele (26)...(0): single nucleotide polymorphism
gagccgtgtggctgtggcctccgggcggcggtggacggcgtgcgcttcatc

SEQ ID NO: 300 - DNA, 51 bp, Homo sapiens
  allele (26)...(0): single nucleotide polymorphism
gggtgcacggccggccctgggcaggcgtagccatggagctgtggcgccaat

SEQ ID NO: 301 - DNA, 51 bp, Homo sapiens
  allele (26)...(0): single nucleotide polymorphism
atggggccggtgtctcgccaggaggcgcagacccggctccagggccagcgc

SEQ ID NO: 302 - DNA, 51 bp, Homo sapiens
  allele (26)...(0): single nucleotide polymorphism
agcatttgaggaagcataactgacgtgtgaagggggtgtggggtacttgcc

SEQ ID NO: 303 - DNA, 51 bp, Homo sapiens
  allele (26)...(0): single nucleotide polymorphism
agcatctgcagacgacccccgcagcctttccctcggacccccctcgaagcc

SEQ ID NO: 304 - DNA, 47 bp, Homo sapiens
  allele (22)...(0): single nucleotide polymorphism
cactgctgtgcagggcagggatgctccaggcagacagcccagcaaag

SEQ ID NO: 305 - DNA, 50 bp, Homo sapiens
  allele (26)...(0): single nucleotide polymorphism
cagcacagcgagcgctctcattctgccttttttcctcttctcagccaact

SEQ ID NO: 306 - DNA, 51 bp, Homo sapiens
  allele (26)...(0): single nucleotide polymorphism
tctgtagagctctgaaaaggttgacgatatagaggtcttgtatgtttttac

SEQ ID NO: 307 - DNA, 51 bp, Homo sapiens
  allele (26)...(0): single nucleotide polymorphism
gacagacgagacagtgaggtatgtggggctgctccggaatggtccggaggc

SEQ ID NO: 308 - DNA, 51 bp, Homo sapiens
  allele (26)...(0): single nucleotide polymorphism
taactatgcaagacaagacttggtcgtcacgttcgcgtctctagttgattt

SEQ ID NO: 309 - DNA, 51 bp, Homo sapiens
  allele (26)...(0): single nucleotide polymorphism
aattaaaactctaggtgtatacttacatggaactagtttatttcctattta

SEQ ID NO: 310 - DNA, 51 bp, Homo sapiens
  allele (26)...(0): single nucleotide polymorphism
tgctcgcgccgtgccactaaggtcattcccgcctccgagagcccagagccg

SEQ ID NO: 311 - DNA, 51 bp, Homo sapiens
  allele (26)...(0): single nucleotide polymorphism
cttttccctcttaccctctctctcttaacatcgtaaacaacagacttacgt

SEQ ID NO: 312 - DNA, 51 bp, Homo sapiens
  allele (26)...(0): single nucleotide polymorphism
cctcttaccctctctctctgaacattgtaaacaacagacttacgttaaact

SEQ ID NO: 313 - DNA, 51 bp, Homo sapiens
  allele (26)...(0): single nucleotide polymorphism
caaaatgtaacagtggcttttcaacgggagtaaagcaaagtctctaaagct

SEQ ID NO: 314 - DNA, 51 bp, Homo sapiens
  allele (26)...(0): single nucleotide polymorphism
aaagatgtttgaatacttaaacactatcacaagatggcaaaatgctgaaag

SEQ ID NO: 315 - DNA, 51 bp, Homo sapiens
  allele (26)...(0): single nucleotide polymorphism
tggtggagccactgcagtgttatctcaaaataagaatattttgttgagata

SEQ ID NO: 316 - DNA, 51 bp, Homo sapiens
  allele (26)...(0): single nucleotide polymorphism
cacttaacttgcatgtgcacagcttctggtaacaaatatcgctaaacctta

SEQ ID NO: 317 - DNA, 51 bp, Homo sapiens
  allele (26)...(0): single nucleotide polymorphism
atgtgattaattatgtgatgaaaactttttttataaatgatcttggtctat

SEQ ID NO: 318 - DNA, 51 bp, Homo sapiens
  allele (26)...(0): single nucleotide polymorphism
caatcagaattgataagcactgttcttccactccatttagcaattatgtca

SEQ ID NO: 319 - DNA, 51 bp, Homo sapiens
  allele (26)...(0): single nucleotide polymorphism
tccatccctcttttgggctcttctgcagggaagtaacatttactgagcacc

SEQ ID NO: 320 - DNA, 51 bp, Homo sapiens
  allele (26)...(0): single nucleotide polymorphism
tacccggaagttgagctcaatttcacttctgttttctggccacaactgcca

SEQ ID NO: 321 - DNA, 51 bp, Homo sapiens
  allele (26)...(0): single nucleotide polymorphism
cccagtcctgcggctcctactggggcgtgcgctggtcggaagattgctgga

SEQ ID NO: 322 - DNA, 51 bp, Homo sapiens
  allele (26)...(0): single nucleotide polymorphism
tactggggagtgcgctggtcggaaggattgctggactcgctgaagagagac

SEQ ID NO: 323 - DNA, 51 bp, Homo sapiens
  allele (26)...(0): single nucleotide polymorphism
cggtccgtggtccccgggggcgcaggtcgcagcgctcccgccctccaggcg

SEQ ID NO: 324 - DNA, 51 bp, Homo sapiens
  allele (26)...(0): single nucleotide polymorphism
ttctcaaaaggctgggggtatttatgtaagaacttattccaaagtgactct

SEQ ID NO: 325 - DNA, 50 bp, Homo sapiens
  allele (26)...(0): single nucleotide polymorphism
aggaaagccggagaattggggcacgaagagggggggctttgatgacccgc

SEQ ID NO: 326 - DNA, 51 bp, Homo sapiens
  allele (26)...(0): single nucleotide polymorphism
agattcatcagaataggatttttgccaaatcccacccatatgctgttgagc

SEQ ID NO: 327 - DNA, 51 bp, Homo sapiens
  allele (26)...(0): single nucleotide polymorphism
ccgctgtctctgtcttcgctttttattcaagaagaataatgcgacgaaaat

SEQ ID NO: 328 - DNA, 51 bp, Homo sapiens
  allele (26)...(0): single nucleotide polymorphism
ccacttctctgggacacattgccttttgttttctccagcatgcgcttgctc

SEQ ID NO: 329 - DNA, 51 bp, Homo sapiens
  allele (26)...(0): single nucleotide polymorphism
catcatcatcatagtttacttcagctcttaaatccccgaggagtctgccct

SEQ ID NO: 330 - DNA, 51 bp, Homo sapiens
  allele (26)...(0): single nucleotide polymorphism
ctcttgcccagccggctgcaagttttgtaagcgcgggacagacactgctga

SEQ ID NO: 331 - DNA, 51 bp, Homo sapiens
  allele (26)...(0): single nucleotide polymorphism
aggttaccaaacaggaatacaacacttctctcccttttctgctctagaagg

SEQ ID NO: 332 - DNA, 51 bp, Homo sapiens
  allele (26)...(0): single nucleotide polymorphism
tgggtgatgatcactgtgctgcttgcggctcatggcagagcattcagtgcc

SEQ ID NO: 333 - DNA, 51 bp, Homo sapiens
  allele (26)...(0): single nucleotide polymorphism
acagactggctgcagcattaggaattaggtcattccgaaactcatcattga

SEQ ID NO: 334 - DNA, 51 bp, Homo sapiens
  allele (26)...(0): single nucleotide polymorphism
ggtcattccgaaactcatcattgaaccaggaagaagaagagttcaatctta

SEQ ID NO: 335 - DNA, 51 bp, Homo sapiens
  allele (26)...(0): single nucleotide polymorphism
agaatggcactgaattcgtttcttcgaacacagatataattgttggttcaa

SEQ ID NO: 336 - DNA, 51 bp, Homo sapiens
  allele (26)...(0): single nucleotide polymorphism
ctttcacttggtgctggagaattcagaagtcaagaacatgctaagcataag

SEQ ID NO: 337 - DNA, 51 bp, Homo sapiens
  allele (26)...(0): single nucleotide polymorphism
ttcaaaagtcaagaacatgctaagcgtaagggacccaaggtagaaagagat

SEQ ID NO: 338 - DNA, 51 bp, Homo sapiens
  allele (26)...(0): single nucleotide polymorphism
ttctccttccagaatgaggccctgggaggaccctcctagtgatctgttact

SEQ ID NO: 339 - DNA, 51 bp, Homo sapiens
  allele (26)...(0): single nucleotide polymorphism
actacataaggacagcaacatgcctgtggacatgagagaatttgtcttact

SEQ ID NO: 340 - DNA, 51 bp, Homo sapiens
  allele (26)...(0): single nucleotide polymorphism
gaaaaaaatcacaaggcaactgtgagtccgggaatctcttctctgatcctt

SEQ ID NO: 341 - DNA, 51 bp, Homo sapiens
  allele (26)...(0): single nucleotide polymorphism
tccgaccccacacaccctgagggaggcctaccctagcctcagccgctcctg

SEQ ID NO: 342 - DNA, 51 bp, Homo sapiens
  allele (26)...(0): single nucleotide polymorphism
ataatccatgcctctgaatattagagtggtttcttggatgggattttgaat

SEQ ID NO: 343 - DNA, 51 bp, Homo sapiens
  allele (26)...(0): single nucleotide polymorphism
gggattttgaatatgcatttaagaacgttgggaagaatttcacagatgatg

SEQ ID NO: 344 - DNA, 51 bp, Homo sapiens
  allele (26)...(0): single nucleotide polymorphism
gaatatgcatttaagaagttgggaacaatttcacagatgatgattggagga

SEQ ID NO: 345 - DNA, 51 bp, Homo sapiens
  allele (26)...(0): single nucleotide polymorphism
tcggcaaatcttgaaagctgcagggtgcagagacatggatgtgacttccca

SEQ ID NO: 346 - DNA, 51 bp, Homo sapiens
  allele (26)...(0): single nucleotide polymorphism
aaggcataagaactaggagctgctggacatttcaatatgaagggcaactcc

SEQ ID NO: 347 - DNA, 51 bp, Homo sapiens
  allele (26)...(0): single nucleotide polymorphism
gaatgtggggataaggcattgggactctatcaggtatcctgaggagagact

SEQ ID NO: 348 - DNA, 51 bp, Homo sapiens
  allele (26)...(0): single nucleotide polymorphism
cagccgggagctctgccagctttggtgaaggagggtgcttgcctcgtgccc

SEQ ID NO: 349 - DNA, 51 bp, Homo sapiens
  allele (26)...(0): single nucleotide polymorphism
cgggagctctgccagctttggcgaacgagggtgcttgcctcgtgccccttg

SEQ ID NO: 350 - DNA, 51 bp, Homo sapiens
  allele (26)...(0): single nucleotide polymorphism
tgctcttgctgctgatggaggaggagggggtggatcccgtggagcctccaa

SEQ ID NO: 351 - DNA, 51 bp, Homo sapiens
  allele (26)...(0): single nucleotide polymorphism
atgcttcccccaaccctagggaatccacacttaagataattcgccacttct

SEQ ID NO: 352 - DNA, 51 bp, Homo sapiens
  allele (26)...(0): single nucleotide polymorphism
ccaaccctagggaatcaacacttaatataattcgccacttctcctctttct

SEQ ID NO: 353 - DNA, 51 bp, Homo sapiens
  allele (26)...(0): single nucleotide polymorphism
agataattcgccacttctcctctttttctctgctccgctcacggcttgcag

SEQ ID NO: 354 - DNA, 51 bp, Homo sapiens
  allele (26)...(0): single nucleotide polymorphism
cgcagagccccgccgtgggtccgcccgctgaggcgcccccagccagtgcgc

SEQ ID NO: 355 - DNA, 51 bp, Homo sapiens
  allele (26)...(0): single nucleotide polymorphism
cagcgccttcttgctggcacccaatggaagccatgcgccggaccacgacgt

SEQ ID NO: 356 - DNA, 51 bp, Homo sapiens
  allele (26)...(0): single nucleotide polymorphism
tgcgccggaccacgacgtcacgcaggaaagggacgaggtgtgggtggtggg

SEQ ID NO: 357 - DNA, 51 bp, Homo sapiens
  allele (26)...(0): single nucleotide polymorphism
gcaggtcttctttgaaggcctatggcaatggctactccagcaacggcaaca

SEQ ID NO: 358 - DNA, 51 bp, Homo sapiens
  allele (26)...(0): single nucleotide polymorphism
attgtagtacaaatgactcactgctataaagcagtttttctacttttaaag

SEQ ID NO: 359 - DNA, 51 bp, Homo sapiens
  allele (26)...(0): single nucleotide polymorphism
ataaacttagaataaaattgtaaaaattgtatagagatatgcagaaggaag

SEQ ID NO: 360 - DNA, 51 bp, Homo sapiens
  allele (26)...(0): single nucleotide polymorphism
aggggtggaactgctgatgggattttccttcattcccttctgataaaggta

SEQ ID NO: 361 - DNA, 51 bp, Homo sapiens
  allele (26)...(0): single nucleotide polymorphism
agcctccccagagacaacaccgggaccctcatctctctcctcaccctgctg

SEQ ID NO: 362 - DNA, 51 bp, Homo sapiens
  allele (26)...(0): single nucleotide polymorphism
gtcttctccgcgcccaccccgctggtaaggggaagtgggcgaagctggagc

SEQ ID NO: 363 - DNA, 51 bp, Homo sapiens
  allele (26)...(0): single nucleotide polymorphism
ggggccgggcactgcccaggaaggggctccgggagagggagccggcggctg

SEQ ID NO: 364 - DNA, 51 bp, Homo sapiens
  allele (26)...(0): single nucleotide polymorphism
agacgaagaccccaggaagtcatcccgcaatgggagagacacgaacaaacc

SEQ ID NO: 365 - DNA, 51 bp, Homo sapiens
  allele (26)...(0): single nucleotide polymorphism
cccacgcctgccaggagcaagccgaagagccagccggccggcgcactccga

SEQ ID NO: 366 - DNA, 51 bp, Homo sapiens
  allele (26)...(0): single nucleotide polymorphism
aaacaaataagccctttttactgacgatgcacccaaccttttcagctgaag

SEQ ID NO: 367 - DNA, 51 bp, Homo sapiens
  allele (26)...(0): single nucleotide polymorphism
agagtcaaaaatccaagtttggattgtaagcagccttgacagtaatcactg

SEQ ID NO: 368 - DNA, 51 bp, Homo sapiens
  allele (26)...(0): single nucleotide polymorphism
aagcagccttgacagtaatcactgagtggtagggaaaaaaagacagttggg

SEQ ID NO: 369 - DNA, 51 bp, Homo sapiens
  allele (26)...(0): single nucleotide polymorphism
aggcaaaagctcacagtaaatgtatcccagaacaggggcctaagtgaaggt

SEQ ID NO: 370 - DNA, 51 bp, Homo sapiens
  allele (26)...(0): single nucleotide polymorphism
ctgctcccancttcgccagcctccagtgtacaacttccgcgtgtagtgggc

SEQ ID NO: 371 - DNA, 51 bp, Homo sapiens
  allele (26)...(0): single nucleotide polymorphism
ctgctcccaacttcgccagcctccagtgtacaacttccgcgtgtagtgggc

SEQ ID NO: 372 - DNA, 51 bp, Homo sapiens
  allele (26)...(0): single nucleotide polymorphism
cacttcactgaaagacaccatttatatacccaagggcagaaagtagaactt

SEQ ID NO: 373 - DNA, 51 bp, Homo sapiens
  allele (26)...(0): single nucleotide polymorphism
gataggactcaagcttatttgggattctgatcaattctttctgatgttgtt

SEQ ID NO: 374 - DNA, 51 bp, Homo sapiens
  allele (26)...(0): single nucleotide polymorphism
tacagccatctgtacctactggagctgcagaagggaagtccactcagtcac

SEQ ID NO: 375 - DNA, 51 bp, Homo sapiens
  allele (26)...(0): single nucleotide polymorphism
agcagtgcagccccggcgcggagcaaggagcctcggcccgcgcccggcgcc

SEQ ID NO: 376 - DNA, 51 bp, Homo sapiens
  allele (26)...(0): single nucleotide polymorphism
gagaaaaagcatggtacccaaccgattttccacttttcagcaatacttcac

SEQ ID NO: 377 - DNA, 51 bp, Homo sapiens
  allele (26)...(0): single nucleotide polymorphism
taaagttttaagaaatgtcataatgtcatgagcttgaaatatctctaggca

SEQ ID NO: 378 - DNA, 51 bp, Homo sapiens
  allele (26)...(0): single nucleotide polymorphism
agcaaagaaacactggcagaattcctgcatttgcaaaattctaagttttgg

SEQ ID NO: 379 - DNA, 51 bp, Homo sapiens
  allele (26)...(0): single nucleotide polymorphism
aaataaatgttttcatagtcattacactttacaatgggagtgctaaaattc

SEQ ID NO: 380 - DNA, 51 bp, Homo sapiens
  allele (26)...(0): single nucleotide polymorphism
aactgggttgctctaagaactgatgcctaaaccgtctcagcatggcctgta

SEQ ID NO: 381 - DNA, 50 bp, Homo sapiens
  allele (26)...(0): single nucleotide polymorphism
caatgcatgaatctgtacccttcggagggcactcacatgccgcccccagc

SEQ ID NO: 382 - DNA, 50 bp, Homo sapiens
  allele (26)...(0): single nucleotide polymorphism
ttgttcatgatttcttgatgttccttaatggaaaactaagagatggaatt

SEQ ID NO: 383 - DNA, 51 bp, Homo sapiens
  allele (26)...(0): single nucleotide polymorphism
gccgagtccgctggtgggcggaccctaggggagcagccagtagggaagttg

SEQ ID NO: 384 - DNA, 51 bp, Homo sapiens
  allele (26)...(0): single nucleotide polymorphism
ccgagtccgctggtgggcggacccatggggagcagccagtagggaagttgg

SEQ ID NO: 385 - DNA, 51 bp, Homo sapiens
  allele (26)...(0): single nucleotide polymorphism
gggagcagccagtagggaagttgggggagttccagaatcagggggcgtggc

SEQ ID NO: 386 - DNA, 51 bp, Homo sapiens
  allele (26)...(0): single nucleotide polymorphism
taatcgggagggctggagcagaggggggccccgccgaggggcgtggtcagt

SEQ ID NO: 387 - DNA, 51 bp, Homo sapiens
  allele (26)...(0): single nucleotide polymorphism
gatgccaaaaaaacaaaggtgagaacccacaacacaggtctaaactcagca

SEQ ID NO: 388 - DNA, 50 bp, Homo sapiens
  allele (26)...(0): single nucleotide polymorphism
gtcttttacagatggtttttcaaaaagagtccagtaaaatatttcacatt

SEQ ID NO: 389 - DNA, 51 bp, Homo sapiens
  allele (26)...(0): single nucleotide polymorphism
cgttgttcctaatgtggatctaccatccctgtgttcatcgagattccggtc

SEQ ID NO: 390 - DNA, 51 bp, Homo sapiens
  allele (26)...(0): single nucleotide polymorphism
tgggattacaggtgcgcactaccacgccaagctaatttttgtattttttag

SEQ ID NO: 391 - DNA, 51 bp, Homo sapiens
  allele (26)...(0): single nucleotide polymorphism
ccttcagcacccctgcagcggaaaataatgagccgccgtagccgccatccg

SEQ ID NO: 392 - DNA, 51 bp, Homo sapiens
  allele (26)...(0): single nucleotide polymorphism
aaaaagctacagaaaagaaatcactctgaaaaacacaatgactcagaggca

SEQ ID NO: 393 - DNA, 50 bp, Homo sapiens
  allele (26)...(0): single nucleotide polymorphism
cagggacatgcgggcaccccgtgggtctttggcggctcacaggacaatgg

SEQ ID NO: 394 - DNA, 50 bp, Homo sapiens
  allele (26)...(0): single nucleotide polymorphism
gcaggcagagcaccctgggaccccaggcagaaggacccctgccctccagt

SEQ ID NO: 395 - DNA, 51 bp, Homo sapiens
  allele (26)...(0): single nucleotide polymorphism
taaacagctcagttcagggactggtgtacaagctggccacccatctcagcc

SEQ ID NO: 396 - DNA, 51 bp, Homo sapiens
  allele (26)...(0): single nucleotide polymorphism
ttacaggacatcacctgccatcttaaggtttaatatttacaaatgcctagt

SEQ ID NO: 397 - DNA, 51 bp, Homo sapiens
  allele (26)...(0): single nucleotide polymorphism
ggccgatttttccacaatttaaatctcagttcacctggtatccagctccag

SEQ ID NO: 398 - DNA, 51 bp, Homo sapiens
  allele (26)...(0): single nucleotide polymorphism
gtttccacctccccagacaggcatttcgagtgggaggcgggagcacgtacc

SEQ ID NO: 399 - DNA, 51 bp, Homo sapiens
  allele (26)...(0): single nucleotide polymorphism
tttccacctccccagacaggcattctgagtgggaggcgggagcacgtaccg

SEQ ID NO: 400 - DNA, 51 bp, Homo sapiens
  allele (26)...(0): single nucleotide polymorphism
ctaaacccaaatgggggctgctggctgaccccgagggtgcctggccagtcc

SEQ ID NO: 401 - DNA, 51 bp, Homo sapiens
  allele (26)...(0): single nucleotide polymorphism
ttttatcattaaagtgccagaatggttctttaatgaaaacaaaaaacaaag

SEQ ID NO: 402 - DNA, 51 bp, Homo sapiens
  allele (26)...(0): single nucleotide polymorphism
ggagggttggagtcactgacgaatgtgagccgggccaggcccatgcaaagg

SEQ ID NO: 403 - DNA, 50 bp, Homo sapiens
  allele (26)...(0): single nucleotide polymorphism
gccacctgcccgggctgtggaggaggctcgcgctgaccaggcgctggggc

SEQ ID NO: 404 - DNA, 51 bp, Homo sapiens
  allele (26)...(0): single nucleotide polymorphism
gcttctgcccacaccgcagggacaagccctggagaaatgggagcntgggga

SEQ ID NO: 405 - DNA, 51 bp, Homo sapiens
  allele (26)...(0): single nucleotide polymorphism
catttctctttgtacataatacatttacctccctgcctcctctcctttcta

SEQ ID NO: 406 - DNA, 51 bp, Homo sapiens
  allele (26)...(0): single nucleotide polymorphism
caggggtcagcagagcttcagaggttgccccacctgagcccccacccggga

SEQ ID NO: 407 - DNA, 51 bp, Homo sapiens
  allele (26)...(0): single nucleotide polymorphism
cagaaagcagcaaattagtgtttttaaggaccgaattcggctcccgcagct

SEQ ID NO: 408 - DNA, 51 bp, Homo sapiens
  allele (26)...(0): single nucleotide polymorphism
aagcagcaaattagtgtttttcaggcccgaattcggctcccgcagctcctg

SEQ ID NO: 409 - DNA, 51 bp, Homo sapiens
  allele (26)...(0): single nucleotide polymorphism
ctcccgcagctcctgcatctccatttgtctagattttatttcttctttgca

SEQ ID NO: 410 - DNA, 51 bp, Homo sapiens
  allele (26)...(0): single nucleotide polymorphism
cccgcccagcccgacgcctactgagtccccgcgctcgccccaccggcgcgc

SEQ ID NO: 411 - DNA, 51 bp, Homo sapiens
  allele (26)...(0): single nucleotide polymorphism
ccagcccgacgcctactgagccccgtgctcgccccaccggcgcgctcttcg

SEQ ID NO: 412 - DNA, 51 bp, Homo sapiens
  allele (26)...(0): single nucleotide polymorphism
agcccgacgcctactgagccccgcgttcgccccaccggcgcgctcttcgcg

SEQ ID NO: 413 - DNA, 51 bp, Homo sapiens
  allele (26)...(0): single nucleotide polymorphism
cgacgcctactgagccccgcgctcgtcccaccggcgcgctcttcgcgcccg

SEQ ID NO: 414 - DNA, 51 bp, Homo sapiens
  allele (26)...(0): single nucleotide polymorphism
gctacgtttactcacagccagcgaaactgacattaaaataactaacaaaca

SEQ ID NO: 415 - DNA, 51 bp, Homo sapiens
  allele (26)...(0): single nucleotide polymorphism
cgcctctgatccaagccacctcccgtcagagaggtgtcatgggcttccaaa

SEQ ID NO: 416 - DNA, 50 bp, Homo sapiens
  allele (26)...(0): single nucleotide polymorphism
ctctgcacaagggaagcctatcctatttttttttcctttgcgaaaacaga

SEQ ID NO: 417 - DNA, 50 bp, Homo sapiens
  allele (26)...(0): single nucleotide polymorphism
aatgcctcagatcagtgacccaaggaccttccagaatggatgaaatagac

SEQ ID NO: 418 - DNA, 50 bp, Homo sapiens
  allele (26)...(0): single nucleotide polymorphism
atgcctcagatcagtgacccaaggaccttccagaatggatgaaatagacc

SEQ ID NO: 419 - DNA, 51 bp, Homo sapiens
  allele (26)...(0): single nucleotide polymorphism
ccaagcggaaggccattttccctgctcttcctcagttgtccggggcggggg

SEQ ID NO: 420 - DNA, 51 bp, Homo sapiens
  allele (26)...(0): single nucleotide polymorphism
ctaattgtgtcgaatttccaggattagaggaaaagttgctccctttcagcc

SEQ ID NO: 421 - DNA, 51 bp, Homo sapiens
  allele (26)...(0): single nucleotide polymorphism
aaagcaatcacagtgttaaaagaagacacgttgaaatgatgcaggctgctc

SEQ ID NO: 422 - DNA, 51 bp, Homo sapiens
  allele (26)...(0): single nucleotide polymorphism
ccagccagctcatttcactttacaccctcatggactgagtttatactcacc

SEQ ID NO: 423 - DNA, 51 bp, Homo sapiens
  allele (26)...(0): single nucleotide polymorphism
agccttcccgcagaaaaagatgcagtcccccagaccttctctgtgctgatt

SEQ ID NO: 424 - DNA, 51 bp, Homo sapiens
  allele (26)...(0): single nucleotide polymorphism
taccagaggctgcatcggctgcgcggagagcagatggcgtcgtattttggg

SEQ ID NO: 425 - DNA, 51 bp, Homo sapiens
  allele (26)...(0): single nucleotide polymorphism
tggtgggcgctccactgtatatggacagccgggcagaccgaaaactggccg

SEQ ID NO: 426 - DNA, 51 bp, Homo sapiens
  allele (26)...(0): single nucleotide polymorphism
ctctcaacaggcaggcaccaccctggacctggatctgggcggaaagcacag

SEQ ID NO: 427 - DNA, 51 bp, Homo sapiens
  allele (26)...(0): single nucleotide polymorphism
ggttacaagtgtgaatgtagtcgtgcctatcaaatggatcttgctactggc

SEQ ID NO: 428 - DNA, 51 bp, Homo sapiens
  allele (26)...(0): single nucleotide polymorphism
gccgaggacgtgcgtggcaacctgaagggcaacaccgaggggctgcagaag

SEQ ID NO: 429 - DNA, 51 bp, Homo sapiens
  allele (26)...(0): single nucleotide polymorphism
tacgaggtgcccttggagaccccgcgtgtccacagccgggcaccgtcccca

SEQ ID NO: 430 - DNA, 51 bp, Homo sapiens
  allele (26)...(0): single nucleotide polymorphism
gaggagcccttcggggtgatcgtgcgccggcagctggacggccgcgtgctg

SEQ ID NO: 431 - DNA, 51 bp, Homo sapiens
  allele (26)...(0): single nucleotide polymorphism
gactgactggggaaaggaacctaaactcgagtctgcctggatgaatggaga

SEQ ID NO: 432 - DNA, 51 bp, Homo sapiens
  allele (26)...(0): single nucleotide polymorphism
agttattctagaggatacagaaatcgtcgaagttcccgagaaactagggag

SEQ ID NO: 433 - DNA, 51 bp, Homo sapiens
  allele (26)...(0): single nucleotide polymorphism
ggaccagggggccatgctgctcaatatctcaggccacgtcaaggagagcgg

SEQ ID NO: 434 - DNA, 51 bp, Homo sapiens
  allele (26)...(0): single nucleotide polymorphism
tgcttttcagggcggaaaactctctgttgtcctgcgagctgaagatatccc

SEQ ID NO: 435 - DNA, 51 bp, Homo sapiens
  allele (26)...(0): single nucleotide polymorphism
gtgtgggccttggtgaactctagcaagcggctaatgtctcctggtttggtc

SEQ ID NO: 436 - DNA, 51 bp, Homo sapiens
  allele (26)...(0): single nucleotide polymorphism
ggagatgtggtcattcctagtgatttttttcagatagtgggaggaagcaac

SEQ ID NO: 437 - DNA, 51 bp, Homo sapiens
  allele (26)...(0): single nucleotide polymorphism
aaggagaagagaaagctgtttatccgttccatgggtgaaggtacaataaat

SEQ ID NO: 438 - DNA, 51 bp, Homo sapiens
  allele (26)...(0): single nucleotide polymorphism
gccactgtctcttccaaacccttcaagccttgtcttgcttgttctcgtcta

SEQ ID NO: 439 - DNA, 51 bp, Homo sapiens
  allele (26)...(0): single nucleotide polymorphism
ttccaaatgctgagcccagagcgttatctcctgcccatgagcaccacagtc

SEQ ID NO: 440 - DNA, 51 bp, Homo sapiens
  allele (26)...(0): single nucleotide polymorphism
ctggaacagtttcctcattagccctctgaccccagcacacgcagggaccta

SEQ ID NO: 441 - DNA, 51 bp, Homo sapiens
  allele (26)...(0): single nucleotide polymorphism
tcatcgctggtgctccaaaaaaaaagatgctgctgtaatgaaccaagagcc

SEQ ID NO: 442 - DNA, 51 bp, Homo sapiens
  allele (26)...(0): single nucleotide polymorphism
ggatgctgttgctctcccacagccattgggcgttccaaatgaaagccaagc

SEQ ID NO: 443 - DNA, 51 bp, Homo sapiens
  allele (26)...(0): single nucleotide polymorphism
gccaatggcatccagaacaaggaggtggaggtccgcatctttcactgctgc

SEQ ID NO: 444 - DNA, 51 bp, Homo sapiens
  allele (26)...(0): single nucleotide polymorphism
tctcgactaacagcatttccaaagacggagcgaatattgtccacggttgag

SEQ ID NO: 445 - DNA, 51 bp, Homo sapiens
  allele (26)...(0): single nucleotide polymorphism
gagtgccttgacgatacagctaattgagaatcattttgtggacgaatatga

SEQ ID NO: 446 - DNA, 51 bp, Homo sapiens
  allele (26)...(0): single nucleotide polymorphism
aagaagttatggaattccttttattcaaacatcagcaaagacaagacaggg

SEQ ID NO: 447 - DNA, 51 bp, Homo sapiens
  allele (26)...(0): single nucleotide polymorphism
gccgcctcagccagcaagcaggcggttaggccagtcctagccaccacagag

SEQ ID NO: 448 - DNA, 51 bp, Homo sapiens
  allele (26)...(0): single nucleotide polymorphism
gggatgtactgcatggtgttcttggcgctgtatgtgcaggcacgactctgt

SEQ ID NO: 449 - DNA, 51 bp, Homo sapiens
  allele (26)...(0): single nucleotide polymorphism
tcaggtggtgggaacctaccgttgcgttcctggaaagaagggaggctacac

SEQ ID NO: 450 - DNA, 51 bp, Homo sapiens
  allele (26)...(0): single nucleotide polymorphism
gtacagcgggcgggccacctcgggctctgagcaccaattttgcggggggcg

SEQ ID NO: 451 - DNA, 51 bp, Homo sapiens
  allele (26)...(0): single nucleotide polymorphism
ggatgctggagagtggatcactgtcgatcagacgacaacagccaaccgtta

SEQ ID NO: 452 - DNA, 51 bp, Homo sapiens
  allele (26)...(0): single nucleotide polymorphism
gattcctccagagctggtgttggaacttcccatcaggcaccccaagtttga

SEQ ID NO: 453 - DNA, 51 bp, Homo sapiens
  allele (26)...(0): single nucleotide polymorphism
aagtttgagtggttcaaggacctggcgctgaagtggtacggcctccccgcc

SEQ ID NO: 454 - DNA, 51 bp, Homo sapiens
  allele (26)...(0): single nucleotide polymorphism
aatttctatatcactggggacagaggatatatggataaagatgggtatttc

SEQ ID NO: 455 - DNA, 51 bp, Homo sapiens
  allele (26)...(0): single nucleotide polymorphism
tggaacaagtggatatccgaaaatgtctgcacacacccacagcagttttgg

SEQ ID NO: 456 - DNA, 51 bp, Homo sapiens
  allele (26)...(0): single nucleotide polymorphism
aggagaggtggtgaaggcatttgtgatcctggcctcgcagttcctgtccca

SEQ ID NO: 457 - DNA, 51 bp, Homo sapiens
  allele (26)...(0): single nucleotide polymorphism
gtgatggaccctctcatatatgccttccgcagccaagagatgcggaagacc

SEQ ID NO: 458 - DNA, 51 bp, Homo sapiens
  allele (26)...(0): single nucleotide polymorphism
ttccatctgaggtttataaaccacgtattcaggcaaagtggccagaatggc

SEQ ID NO: 459 - DNA, 51 bp, Homo sapiens
  allele (26)...(0): single nucleotide polymorphism
caagacctagctccccagcagagagtggccccacaacaaaagaggtccagc

SEQ ID NO: 460 - DNA, 51 bp, Homo sapiens
  allele (26)...(0): single nucleotide polymorphism
gacagagctgtaccgtgacattttcgagcaccttcgggatgaatcaggcaa

SEQ ID NO: 461 - DNA, 51 bp, Homo sapiens
  allele (26)...(0): single nucleotide polymorphism
gagggcgatttctactacctgggggcgttcttcggggggtcggtgcaagag

SEQ ID NO: 462 - DNA, 51 bp, Homo sapiens
  allele (26)...(0): single nucleotide polymorphism
agtgtccctcaccatggtcaccctggtcaccctgcctctgcttttccttct

SEQ ID NO: 463 - DNA, 51 bp, Homo sapiens
  allele (26)...(0): single nucleotide polymorphism
cctggaacgcgccttgtacctgctcataaggagggtgctgcacttgggggt

SEQ ID NO: 464 - DNA, 51 bp, Homo sapiens
  allele (26)...(0): single nucleotide polymorphism
gagcacgaggaagccatgaatgcggtctactcaggctacgtctacacgcac

SEQ ID NO: 465 - DNA, 51 bp, Homo sapiens
  allele (26)...(0): single nucleotide polymorphism
agatactttctataagcagtttttacattgtaggaagcagctgaattcaaa

SEQ ID NO: 466 - DNA, 51 bp, Homo sapiens
  allele (26)...(0): single nucleotide polymorphism
acactggaaagcacaacagttggcagttctgtctagaaaataataattgca

SEQ ID NO: 467 - DNA, 51 bp, Homo sapiens
  allele (26)...(0): single nucleotide polymorphism
aacgctgccctgactgagaaaggcatgatgctcgctccactgctggaaccg

SEQ ID NO: 468 - DNA, 51 bp, Homo sapiens
  allele (26)...(0): single nucleotide polymorphism
catccaggacaacttctcggtgactgaagtgcccttcactgagagcgcctg

SEQ ID NO: 469 - DNA, 51 bp, Homo sapiens
  allele (26)...(0): single nucleotide polymorphism
cttcaaccctggtcggagacaacggctcaccatggccatcagaacagtgcg

SEQ ID NO: 470 - DNA, 51 bp, Homo sapiens
  allele (26)...(0): single nucleotide polymorphism
ctgattcttccgttcttcttgacttgtgccaccttgccagccagctgctcg

SEQ ID NO: 471 - DNA, 51 bp, Homo sapiens
  allele (26)...(0): single nucleotide polymorphism
acggccctggagaaccagaagaaggtgaggaagaagaaagtcttgattgcc

SEQ ID NO: 472 - DNA, 51 bp, Homo sapiens
  allele (26)...(0): single nucleotide polymorphism
ggatggtgtctgatgaggagttggatcagatgctggacagtgggcaaagcg

SEQ ID NO: 473 - DNA, 51 bp, Homo sapiens
  allele (26)...(0): single nucleotide polymorphism
ttggggttggcttggtttcaataagcaacggggacacttacaaattgctgc

SEQ ID NO: 474 - DNA, 51 bp, Homo sapiens
  allele (26)...(0): single nucleotide polymorphism
gtgaccgagcccgagtgccgcgaggtctttcaccgccgcgcccgcgccagc

SEQ ID NO: 475 - DNA, 51 bp, Homo sapiens
  allele (26)...(0): single nucleotide polymorphism
cagtgcctcccctgcggccccggggtcaaaggccgctgcttcgggcccagc

SEQ ID NO: 476 - DNA, 51 bp, Homo sapiens
  allele (26)...(0): single nucleotide polymorphism
ggccaactctgctatggacaccagactactctgctgtgcggtcatctgtct

SEQ ID NO: 477 - DNA, 51 bp, Homo sapiens
  allele (26)...(0): single nucleotide polymorphism
gcctggaacaccaggctcctctgccatgtcatgctttgtctcctgggagca

SEQ ID NO: 478 - DNA, 51 bp, Homo sapiens
  allele (26)...(0): single nucleotide polymorphism
agccacccagaccggagactcggccatctacctctgtgctgtggaggccta

SEQ ID NO: 479 - DNA, 51 bp, Homo sapiens
  allele (26)...(0): single nucleotide polymorphism
cggccgtctacctctgtgctgtggacgcctattctaacgactacaagctca

SEQ ID NO: 480 - DNA, 51 bp, Homo sapiens
  allele (26)...(0): single nucleotide polymorphism
cagaacaaaagcaaatggaattggatagcatcctggtggccctgctgcaga

SEQ ID NO: 481 - DNA, 51 bp, Homo sapiens
  allele (26)...(0): single nucleotide polymorphism
gaaacccggaagcactgtaattgcggggtctataaatgcacatggctctgt

SEQ ID NO: 482 - DNA, 51 bp, Homo sapiens
  allele (26)...(0): single nucleotide polymorphism
tgtattcctgtaatggggctgatgatatatatgatggttatggaccaccac

SEQ ID NO: 483 - DNA, 51 bp, Homo sapiens
  allele (26)...(0): single nucleotide polymorphism
gcccctgagcagtcaggacccggcttccgtccgtgagtgccacgatcccag

SEQ ID NO: 484 - DNA, 51 bp, Homo sapiens
  allele (26)...(0): single nucleotide polymorphism
ggagtgggtgctgctgctcttgggagcttgtgctgcccctccagcctgggc

SEQ ID NO: 485 - DNA, 51 bp, Homo sapiens
  allele (26)...(0): single nucleotide polymorphism
cgtgtcctccctcccctatgcggtggccccgctcagcctgccccgagggga

SEQ ID NO: 486 - DNA, 51 bp, Homo sapiens
  allele (26)...(0): single nucleotide polymorphism
ggggtactatgggccccagtgtcagcttgtgattcagtgtgagcctttgga

SEQ ID NO: 487 - DNA, 51 bp, Homo sapiens
  allele (26)...(0): single nucleotide polymorphism
gctgggtaccatggactgtactcactctttgggaaacttcagcttcagctc

SEQ ID NO: 488 - DNA, 51 bp, Homo sapiens
  allele (26)...(0): single nucleotide polymorphism
tgcagaaggcaccacagagaccggagggcagggcaagggcacctcgaagac

SEQ ID NO: 489 - DNA, 51 bp, Homo sapiens
  allele (26)...(0): single nucleotide polymorphism
gtgtgtgtgtaatggtgtggctgtatgctccaaccaagatcttattactga

SEQ ID NO: 490 - DNA, 51 bp, Homo sapiens
  allele (26)...(0): single nucleotide polymorphism
cggaagctggtgtcctactgcccccgaaggttgcaacaactgttgcccctc

SEQ ID NO: 491 - DNA, 51 bp, Homo sapiens
  allele (26)...(0): single nucleotide polymorphism
ccagggcctccaggtccaagaggcccctctggagagcctggtcttccaggg

SEQ ID NO: 492 - DNA, 51 bp, Homo sapiens
  allele (26)...(0): single nucleotide polymorphism
gtgttttacgctgaacgataccaaatgcccacaggcataaaaggcccacta

SEQ ID NO: 493 - DNA, 51 bp, Homo sapiens
  allele (26)...(0): single nucleotide polymorphism
tccaggaataccaggtctgcctggtgttcctggaacaagaggattaaaagg

SEQ ID NO: 494 - DNA, 51 bp, Homo sapiens
  allele (26)...(0): single nucleotide polymorphism
agactgtgttaccaacagaccatgcggaagtcaagtgcgatgtgaaggctt

SEQ ID NO: 495 - DNA, 51 bp, Homo sapiens
  allele (26)...(0): single nucleotide polymorphism
ctccagttctacaacttgtgtaaggcaagcacagtgtggacaggatttcca

SEQ ID NO: 496 - DNA, 51 bp, Homo sapiens
  allele (26)...(0): single nucleotide polymorphism
cagtttgggggacagccatgcactgcgcctctggtagcctttcaaccatgc

SEQ ID NO: 497 - DNA, 51 bp, Homo sapiens
  allele (26)...(0): single nucleotide polymorphism
ccaggctctcccaggatctcatcaccgcgcccccagggcctcagcaacccc

SEQ ID NO: 498 - DNA, 51 bp, Homo sapiens
  allele (26)...(0): single nucleotide polymorphism
ccaagctcccatgacccagacaacgtccttgaagacaagctgggttaactg

SEQ ID NO: 499 - DNA, 51 bp, Homo sapiens
  allele (26)...(0): single nucleotide polymorphism
tccacgtagaagcggaagccgaggtgggagatgtacgcattgatgggaagg

SEQ ID NO: 500 - DNA, 51 bp, Homo sapiens
  allele (26)...(0): single nucleotide polymorphism
ttggtagggacggaactcggggcgctggcggtggcccgagtggagatagga

SEQ ID NO: 501 - DNA, 51 bp, Homo sapiens
  allele (26)...(0): single nucleotide polymorphism
gctggtttgctcccaggaggccaagcagtcagcctactgcccctacagtca

SEQ ID NO: 502 - DNA, 51 bp, Homo sapiens
  allele (26)...(0): single nucleotide polymorphism
aagcatccgaacaatcctcatctttggaagatgccaggagcaattcggaat

SEQ ID NO: 503 - DNA, 51 bp, Homo sapiens
  allele (26)...(0): single nucleotide polymorphism
ccgcaccaacgccgacatcatcgaggccctgaggaagaagggcttcaaggg

SEQ ID NO: 504 - DNA, 51 bp, Homo sapiens
  allele (26)...(0): single nucleotide polymorphism
tccacgaccgggtagagaactacaaaccgcggcagcgcaagctccgcaacc

SEQ ID NO: 505 - DNA, 51 bp, Homo sapiens
  allele (26)...(0): single nucleotide polymorphism
tcgttggagatgacaagttccggagtgagctcggctgtctggatgggaagg

SEQ ID NO: 506 - DNA, 51 bp, Homo sapiens
  allele (26)...(0): single nucleotide polymorphism
agctggagcaacagcaggaacagcatcaggagcagcagcaggagcaggtgc

SEQ ID NO: 507 - DNA, 51 bp, Homo sapiens
  allele (26)...(0): single nucleotide polymorphism
gtttggcatacctggatattttaattcagtggagataaaagacagcccact

SEQ ID NO: 508 - DNA, 51 bp, Homo sapiens
  allele (26)...(0): single nucleotide polymorphism
tattttaatccagtggagataaaagccagcccactaggaagtatatcaata

SEQ ID NO: 509 - DNA, 51 bp, Homo sapiens
  allele (26)...(0): single nucleotide polymorphism
aagtgaattctatcttgcaatgaacgaggaaggaaaactctatgcaaagaa

SEQ ID NO: 510 - DNA, 51 bp, Homo sapiens
  allele (26)...(0): single nucleotide polymorphism
gatgagctctccaaccacgtattttatgcgtttttgatccagacccagatg

SEQ ID NO: 511 - DNA, 51 bp, Homo sapiens
  allele (26)...(0): single nucleotide polymorphism
caccagcaagatgcccacgatcagccgaacctgcccaaggcctgcttcttg

SEQ ID NO: 512 - DNA, 51 bp, Homo sapiens
  allele (26)...(0): single nucleotide polymorphism
ttttccccaggggtcacagactgatgacccacagaggtcagggtcttctgt

SEQ ID NO: 513 - DNA, 51 bp, Homo sapiens
  allele (26)...(0): single nucleotide polymorphism
ggggtcacagactgataacccacaggggtcagggtcttctgtccagtggtc

SEQ ID NO: 514 - DNA, 51 bp, Homo sapiens
  allele (26)...(0): single nucleotide polymorphism
ctgatgacccacagaagtcatggtcgttgccccagtgatctcagtcttctc

SEQ ID NO: 515 - DNA, 51 bp, Homo sapiens
  allele (26)...(0): single nucleotide polymorphism
atatgtgtcatactgggaggtgttgtatgtgaggatgtacacccctgtgtt

SEQ ID NO: 516 - DNA, 51 bp, Homo sapiens
  allele (26)...(0): single nucleotide polymorphism
aaggagcctctctccttccatgtcatctggatcgcatccttttacaaccat

SEQ ID NO: 517 - DNA, 51 bp, Homo sapiens
  allele (26)...(0): single nucleotide polymorphism
attccagcaccatcgttttcctgtgcccctggtccaggggaaacttcagca

SEQ ID NO: 518 - DNA, 51 bp, Homo sapiens
  allele (26)...(0): single nucleotide polymorphism
gtgctccctgatcctcgtgaagcatgtggtagctcaagttatgtggcatct

SEQ ID NO: 519 - DNA, 51 bp, Homo sapiens
  allele (26)...(0): single nucleotide polymorphism
aatgctgcgaaagatatgaatggaacgtctttgcatggaaaagcaataaaa

SEQ ID NO: 520 - DNA, 51 bp, Homo sapiens
  allele (26)...(0): single nucleotide polymorphism
aagtctgagatctgcaagaggaagccgtggaggaacaagagggtggcttcc

SEQ ID NO: 521 - DNA, 51 bp, Homo sapiens
  allele (26)...(0): single nucleotide polymorphism
gcggataagtagaggaccttcatgtggtatttgctggtgaagttggttcgg

SEQ ID NO: 522 - DNA, 51 bp, Homo sapiens
  allele (26)...(0): single nucleotide polymorphism
cttagacatacaatatacttaccttggaggtcacgtatgtttgtccgcaca

SEQ ID NO: 523 - DNA, 51 bp, Homo sapiens
  allele (26)...(0): single nucleotide polymorphism
ggagcgagcgtggatccagttcgcgtcggggttgtttgggtcaagttgctg

SEQ ID NO: 524 - DNA, 51 bp, Homo sapiens
  allele (26)...(0): single nucleotide polymorphism
gccctgtgcctgacggagaggcagagcaagatatggttccagaaccgacgc

SEQ ID NO: 525 - DNA, 51 bp, Homo sapiens
  allele (26)...(0): single nucleotide polymorphism
ctgggcaccatataggatagcccacgccgtcatcaaagcccatgccagagt

SEQ ID NO: 526 - DNA, 51 bp, Homo sapiens
  allele (26)...(0): single nucleotide polymorphism
gtggagggtgcaggtgaagtagcatgcacttccttcttcctctttcttgat

SEQ ID NO: 527 - DNA, 51 bp, Homo sapiens
  allele (26)...(0): single nucleotide polymorphism
tgctccatcaaaaatgaagcaaggctgccatgtttaccgacaccgggaaaa

SEQ ID NO: 528 - DNA, 51 bp, Homo sapiens
  allele (26)...(0): single nucleotide polymorphism
caaaagcaagaaagttctttgagaatttgccagatggcacttggaacttaa

SEQ ID NO: 529 - DNA, 51 bp, Homo sapiens
  allele (26)...(0): single nucleotide polymorphism
agtccaccgccgcctcaggccgtgctgctggccgagtaggagaactggggg

SEQ ID NO: 530 - DNA, 51 bp, Homo sapiens
  allele (26)...(0): single nucleotide polymorphism
ggcactgaagaaatccctgacatcacattggcgctgctgacgggcgtactg

SEQ ID NO: 531 - DNA, 51 bp, Homo sapiens
  allele (26)...(0): single nucleotide polymorphism
gggctgacaaggtgctgattttcacggtggacaaagcgttcccatcgcttt

SEQ ID NO: 532 - DNA, 51 bp, Homo sapiens
  allele (26)...(0): single nucleotide polymorphism
ttcaatgttgtatttgtcaatatagccatataaatcttctgtccccagaac

SEQ ID NO: 533 - DNA, 51 bp, Homo sapiens
  allele (26)...(0): single nucleotide polymorphism
tgtaaaatcgaatatcatagtctgtgaacgtctggtacaattgcttgaagt

SEQ ID NO: 534 - DNA, 51 bp, Homo sapiens
  allele (26)...(0): single nucleotide polymorphism
tcggctaggcagcctccatcctcaccccccttatcacatccgcgtggcatg

SEQ ID NO: 535 - DNA, 51 bp, Homo sapiens
  allele (26)...(0): single nucleotide polymorphism
ttcctcctctattcccggctcgggggccagccagtgtacctgcccactcag

SEQ ID NO: 536 - DNA, 51 bp, Homo sapiens
  allele (26)...(0): single nucleotide polymorphism
tccgggataagctccaggtgctccatggtaggcgcctggaggtgcctgtcc

SEQ ID NO: 537 - DNA, 51 bp, Homo sapiens
  allele (26)...(0): single nucleotide polymorphism
aagcttgtcatgcctcacagcagtgagcacaagactgcccagcccaatgga

SEQ ID NO: 538 - DNA, 51 bp, Homo sapiens
  allele (26)...(0): single nucleotide polymorphism
acttacacctgtgtggtagagcacactggggctcctgagcccatccttcgg

SEQ ID NO: 539 - DNA, 51 bp, Homo sapiens
  allele (26)...(0): single nucleotide polymorphism
ggcctggtgggcttcctcgtgggcatcgtcctcatcatcatgggcacatat

SEQ ID NO: 540 - DNA, 51 bp, Homo sapiens
  allele (26)...(0): single nucleotide polymorphism
ctgagcccagagcgttgtctcctgcgcatgagcaccacagtcaggccttga

SEQ ID NO: 541 - DNA, 51 bp, Homo sapiens
  allele (26)...(0): single nucleotide polymorphism
agcccggccgggccccacggttcgcgcaggagagaacgtgaccttgtcctg

SEQ ID NO: 542 - DNA, 51 bp, Homo sapiens
  allele (26)...(0): single nucleotide polymorphism
ccggctgtgctcaggggtgtggggtgcggatacagaggagcggctggtgga

SEQ ID NO: 543 - DNA, 51 bp, Homo sapiens
  allele (26)...(0): single nucleotide polymorphism
gaagatgccctcctcagacatgagttgaaaggttatcagaaatgggtccgc

SEQ ID NO: 544 - DNA, 51 bp, Homo sapiens
  allele (26)...(0): single nucleotide polymorphism
agatgccctcctcagacatgagtggcaaggttatcagaaatgggtccgccc

SEQ ID NO: 545 - DNA, 51 bp, Homo sapiens
  allele (26)...(0): single nucleotide polymorphism
gcctcgggcttccactacggtgtgctcgcctgcgagggctgcaagggcttt

SEQ ID NO: 546 - DNA, 51 bp, Homo sapiens
  allele (26)...(0): single nucleotide polymorphism
agcgggacggtccggagcaagcccacaggcagaggaggcgacagagggaaa

SEQ ID NO: 547 - DNA, 51 bp, Homo sapiens
  allele (26)...(0): single nucleotide polymorphism
gtgccgggagtgagcgatgagctggtttctgttcctggcccacagagtcgc

SEQ ID NO: 548 - DNA, 51 bp, Homo sapiens
  allele (26)...(0): single nucleotide polymorphism
ggctataatcacaatggggaatggtttgaagcccaaaccaaaaatggccaa

SEQ ID NO: 549 - DNA, 51 bp, Homo sapiens
  allele (26)...(0): single nucleotide polymorphism
atataaacttgtggtagttggagcttgtggcgtaggcaagagtgccttgac

SEQ ID NO: 550 - DNA, 51 bp, Homo sapiens
  allele (26)...(0): single nucleotide polymorphism
tggatattctcgacacagcaggtcacgaggagtacagtgcaatgagggacc

SEQ ID NO: 551 - DNA, 51 bp, Homo sapiens
  allele (26)...(0): single nucleotide polymorphism
ctgttcaggatctcctcattctgactgttctcctgatgtccaaattggttg

SEQ ID NO: 552 - DNA, 51 bp, Homo sapiens
  allele (26)...(0): single nucleotide polymorphism
aggtcctcgcggagctgggtccggggcccgggagggtaggtcagcgcagac

SEQ ID NO: 553 - DNA, 51 bp, Homo sapiens
  allele (26)...(0): single nucleotide polymorphism
ctggcgcactactcggacctgctcctcctggcgggcctggggctgattgag

SEQ ID NO: 554 - DNA, 51 bp, Homo sapiens
  allele (26)...(0): single nucleotide polymorphism
gcacaaggaacggaattgctgtctggtttctgctttaacagcatttgatgc

SEQ ID NO: 555 - DNA, 51 bp, Homo sapiens
  allele (26)...(0): single nucleotide polymorphism
cttcggggaaagttggggatttcactgtagtcaaagatctgggcctgagtt

SEQ ID NO: 556 - DNA, 51 bp, Homo sapiens
  allele (26)...(0): single nucleotide polymorphism
aggtcctcctcgaattgggatggccgaggtgcatcatcatcatcccagagg

SEQ ID NO: 557 - DNA, 51 bp, Homo sapiens
  allele (26)...(0): single nucleotide polymorphism
ctcagaccatgtccttcggatgcacggttacagagcacctggggagcagga

SEQ ID NO: 558 - DNA, 51 bp, Homo sapiens
  allele (26)...(0): single nucleotide polymorphism
cgctttgagacgcagctgggcaccctggcgcagttccccaacacactcctg

SEQ ID NO: 559 - DNA, 51 bp, Homo sapiens
  allele (26)...(0): single nucleotide polymorphism
gggggacgaggccatggagcgcttcggcgaggatgagggcttcattaaaga

SEQ ID NO: 560 - DNA, 51 bp, Homo sapiens
  allele (26)...(0): single nucleotide polymorphism
cattaaagaagaggagaagcccctggcccgcaacgagttccagcgccaggt

SEQ ID NO: 561 - DNA, 51 bp, Homo sapiens
  allele (26)...(0): single nucleotide polymorphism
tggaggggatgctcattttgatgaacatgaaaggtggaccaacaatttcag

SEQ ID NO: 562 - DNA, 51 bp, Homo sapiens
  allele (26)...(0): single nucleotide polymorphism
gatgaaaggtggaccaacaatttcacagagtacaacttacatcgtgttgcg

SEQ ID NO: 563 - DNA, 51 bp, Homo sapiens
  allele (26)...(0): single nucleotide polymorphism
attctacgattccggtttgctccagtgtaaactagcgctcctttccgtaac

SEQ ID NO: 564 - DNA, 51 bp, Homo sapiens
  allele (26)...(0): single nucleotide polymorphism
cgctgccttctcccgaaaggtctgctccttcacgcgttcggcttcccgcag

SEQ ID NO: 565 - DNA, 51 bp, Homo sapiens
  allele (26)...(0): single nucleotide polymorphism
gtgaaggcatttgtggtcctggccttgcagttcctgtcccatgacccagaa

SEQ ID NO: 566 - DNA, 51 bp, Homo sapiens
  allele (26)...(0): single nucleotide polymorphism
gactgtcacagggaaaattcaacgaaccaagcttcgagacaaggagtggaa

SEQ ID NO: 567 - DNA, 51 bp, Homo sapiens
  allele (26)...(0): single nucleotide polymorphism
gcaagaagttgattatatgactcaggctaggggtcagagatcctctctggc

SEQ ID NO: 568 - DNA, 51 bp, Homo sapiens
  allele (26)...(0): single nucleotide polymorphism
aaggccaacaacctgctctacatcacccctgaggccttccagaaccttccc

SEQ ID NO: 569 - DNA, 51 bp, Homo sapiens
  allele (26)...(0): single nucleotide polymorphism
gaatgtcttgagaatccagtgtctctgcagaaagcagtcttccaaacatgc

SEQ ID NO: 570 - DNA, 51 bp, Homo sapiens
  allele (26)...(0): single nucleotide polymorphism
tctctctggagaagatccaacccatgacacaaaacggtcagcacccaacct

SEQ ID NO: 571 - DNA, 51 bp, Homo sapiens
  allele (26)...(0): single nucleotide polymorphism
agtgtctggatgatctttgtggtcattgcatccgttttcacaaatgggctt

SEQ ID NO: 572 - DNA, 51 bp, Homo sapiens
  allele (26)...(0): single nucleotide polymorphism
catcttctccatcaacctcttcagcggcattttcttcctcacgtgcatgag

SEQ ID NO: 573 - DNA, 51 bp, Homo sapiens
  allele (26)...(0): single nucleotide polymorphism
tcttttgtggacatctgcttctcctccaccaccgtccccaagatgctggcc

SEQ ID NO: 574 - DNA, 51 bp, Homo sapiens
  allele (26)...(0): single nucleotide polymorphism
ggccctgagagcaacaccacgggcaccacagccttctccatgcccagctgg

SEQ ID NO: 575 - DNA, 51 bp, Homo sapiens
  allele (26)...(0): single nucleotide polymorphism
tggaagcgtgcatccagtgagaccattgaggcttgagtcttttagtgcctg

SEQ ID NO: 576 - DNA, 51 bp, Homo sapiens
  allele (26)...(0): single nucleotide polymorphism
gaggcgcggggagccaggcctgggccccgggtccccaagacccttgtgctc

SEQ ID NO: 577 - DNA, 51 bp, Homo sapiens
  allele (26)...(0): single nucleotide polymorphism
actcgcacgtggatcctgaggctgtgagaggtaaggaaggctttgccacag

SEQ ID NO: 578 - DNA, 51 bp, Homo sapiens
  allele (26)...(0): single nucleotide polymorphism
cattgacagcgaggcctcctcagccttcttcatggcgaagaagaagacgcc

SEQ ID NO: 579 - DNA, 51 bp, Homo sapiens
  allele (26)...(0): single nucleotide polymorphism
tgacagagctgtaccgtgacattttgcagcaccttcgggatgaatcaggca

SEQ ID NO: 580 - DNA, 51 bp, Homo sapiens
  allele (26)...(0): single nucleotide polymorphism
cactactatgtcttcaccgaccagctggccgcggtgccccgcgtgacgctg

SEQ ID NO: 581 - DNA, 51 bp, Homo sapiens
  allele (26)...(0): single nucleotide polymorphism
tcggcagctgtcagtgctggaggtgggcgcctacaagcgctggcaggacgt

SEQ ID NO: 582 - DNA, 51 bp, Homo sapiens
  allele (26)...(0): single nucleotide polymorphism
ggtgtgcgtggacgtggacatggagatccgcgaccacgtgggcgtggagat

SEQ ID NO: 583 - DNA, 51 bp, Homo sapiens
  allele (26)...(0): single nucleotide polymorphism
tccgctgttcggcaccctgcaccccagcttctacggaagcagccgggaggc

SEQ ID NO: 584 - DNA, 51 bp, Homo sapiens
  allele (26)...(0): single nucleotide polymorphism
caaggacgagggcgatttctactacatgggggggttcttcggggggtcggt

SEQ ID NO: 585 - DNA, 51 bp, Homo sapiens
  allele (26)...(0): single nucleotide polymorphism
gttcttcggggggtcggtgcaagagatgcagcggctcaccagggcctgcca

SEQ ID NO: 586 - DNA, 51 bp, Homo sapiens
  allele (26)...(0): single nucleotide polymorphism
cacatcgtggtggagctgacccaggatgacgctttgggctccaggtggcgg

SEQ ID NO: 587 - DNA, 51 bp, Homo sapiens
  allele (26)...(0): single nucleotide polymorphism
gtctgaaagatttccacaaggacatgctgaagccctcaccagggaagagcc

SEQ ID NO: 588 - DNA, 51 bp, Homo sapiens
  allele (26)...(0): single nucleotide polymorphism
caagttcaccaacaactgctacaggcacgcgattgtcaccacctccatcaa

SEQ ID NO: 589 - DNA, 51 bp, Homo sapiens
  allele (26)...(0): single nucleotide polymorphism
tcctccggcttcgtcgtcttctcctccctggggtacatggcacagaagcac

SEQ ID NO: 590 - DNA, 51 bp, Homo sapiens
  allele (26)...(0): single nucleotide polymorphism
ctgcggtagctgtcccaggcctcgggccgcgccgcctcgtccatgttgagg

SEQ ID NO: 591 - DNA, 51 bp, Homo sapiens
  allele (26)...(0): single nucleotide polymorphism
gcataggacatggcgggcttgccccgcgcagagctctgggggctactgcta

SEQ ID NO: 592 - DNA, 51 bp, Homo sapiens
  allele (26)...(0): single nucleotide polymorphism
caacccctagaagacctggctggctggaaagagctcttccagacaccagta

SEQ ID NO: 593 - DNA, 51 bp, Homo sapiens
  allele (26)...(0): single nucleotide polymorphism
ccattgttcaagacatcctacgtttggaaatgcctgcaagcaaaattgtcc

SEQ ID NO: 594 - DNA, 51 bp, Homo sapiens
  allele (26)...(0): single nucleotide polymorphism
acaattcagagagggagactgagcatacaccagcattgatcatggtgccaa

SEQ ID NO: 595 - DNA, 51 bp, Homo sapiens
  allele (26)...(0): single nucleotide polymorphism
atcaggaaaggtgttggatcactggtgcatcatgaccagtgaggaagaagt

SEQ ID NO: 596 - DNA, 51 bp, Homo sapiens
  allele (26)...(0): single nucleotide polymorphism
ccccaaacaggaagtccatgggccctaccctgacagcagcttcttaacttc

SEQ ID NO: 597 - DNA, 51 bp, Homo sapiens
  allele (26)...(0): single nucleotide polymorphism
cccaaacaggaagtccatgggcccatccctgacagcagcttcttaacttcc

SEQ ID NO: 598 - DNA, 51 bp, Homo sapiens
  allele (26)...(0): single nucleotide polymorphism
ctggaagaactttgccatgagaaaggaattttggagaagtacggacattca

SEQ ID NO: 599 - DNA, 51 bp, Homo sapiens
  allele (26)...(0): single nucleotide polymorphism
aatgattaacaacaacctgagacacacggatgaaatgttctggaaccacgt

SEQ ID NO: 600 - DNA, 51 bp, Homo sapiens
  allele (26)...(0): single nucleotide polymorphism
gcccccaggcatggctagctcgtgttccgtgcaggtgaagctggagctggg

SEQ ID NO: 601 - DNA, 51 bp, Homo sapiens
  allele (26)...(0): single nucleotide polymorphism
cagctttccatccatttttatttatagacatactgctagtggaaagaccta

SEQ ID NO: 602 - DNA, 51 bp, Homo sapiens
  allele (26)...(0): single nucleotide polymorphism
caggtgtcctgcgagccacccggggctccgggtggcgggggtggcggcggc

SEQ ID NO: 603 - DNA, 51 bp, Homo sapiens
  allele (26)...(0): single nucleotide polymorphism
agaggagagagccgccctcgagcggggcaaggcgattgagaaaaacctcaa

SEQ ID NO: 604 - DNA, 51 bp, Homo sapiens
  allele (26)...(0): single nucleotide polymorphism
gccagagttgcagcatcagggccagcctgagcaggagacccccagtcccat

SEQ ID NO: 605 - DNA, 51 bp, Homo sapiens
  allele (26)...(0): single nucleotide polymorphism
taggaatgacagcagtagcagtaatgggaaggccaaaaatccccctggaga

SEQ ID NO: 606 - DNA, 51 bp, Homo sapiens
  allele (26)...(0): single nucleotide polymorphism
tgttcctggagcctcaatggtacagcgtgctcgagaaggacagtgtgactc

SEQ ID NO: 607 - DNA, 51 bp, Homo sapiens
  allele (26)...(0): single nucleotide polymorphism
gggcacagaaacacagcagcgggagsagcaacaccagcactgccaacagat

SEQ ID NO: 608 - DNA, 51 bp, Homo sapiens
  allele (26)...(0): single nucleotide polymorphism
attgccattgtggtaactctgggtcgcatcatcttcagtgccccaattgtg

SEQ ID NO: 609 - DNA, 51 bp, Homo sapiens
  allele (26)...(0): single nucleotide polymorphism
gtgaagcggtgtatggggacagtgaacctcaaccaggccaggggctccttt

SEQ ID NO: 610 - DNA, 51 bp, Homo sapiens
  allele (26)...(0): single nucleotide polymorphism
agtgacttcagtaaactcttgggtccactttctgccaaaaagtaccttgag

SEQ ID NO: 611 - DNA, 51 bp, Homo sapiens
  allele (26)...(0): single nucleotide polymorphism
cggtataacgtcaaaaatcctgtttttcagccaaggttcagaaattgcctc

SEQ ID NO: 612 - DNA, 51 bp, Homo sapiens
  allele (26)...(0): single nucleotide polymorphism
aaggcgctatgtacagcctcctgaagtgattgggcctatgcggcccgagca

SEQ ID NO: 613 - DNA, 51 bp, Homo sapiens
  allele (26)...(0): single nucleotide polymorphism
tgaagatggtcctgatgggcaggaggtggacccgccaaatccagaggaggt

SEQ ID NO: 614 - DNA, 51 bp, Homo sapiens
  allele (26)...(0): single nucleotide polymorphism
attactgaagggtggagaacagaagcgtcatgaaaaaatatctgcttcatt

SEQ ID NO: 615 - DNA, 51 bp, Homo sapiens
  allele (26)...(0): single nucleotide polymorphism
cacttcctctttctctttggatgcctcaccctcctgttggggggcagatgg

SEQ ID NO: 616 - DNA, 51 bp, Homo sapiens
  allele (26)...(0): single nucleotide polymorphism
gaagacaaggtggtacaaagccctcaatctctggttgtccacgagggagac

SEQ ID NO: 617 - DNA, 51 bp, Homo sapiens
  allele (26)...(0): single nucleotide polymorphism
tgtaactctcaattgcagttatgaaatgactaactttcgaagcctactatg

SEQ ID NO: 618 - DNA, 51 bp, Homo sapiens
  allele (26)...(0): single nucleotide polymorphism
gaagtgactaactttcgaagcctacaatggtacaagcaggaaaagaaagct

SEQ ID NO: 619 - DNA, 51 bp, Homo sapiens
  allele (26)...(0): single nucleotide polymorphism
agcatattagataagaaagaactttccagcatcctgaacatcacagccacc

SEQ ID NO: 620 - DNA, 51 bp, Homo sapiens
  allele (26)...(0): single nucleotide polymorphism
tacagaatatgtcgtcggtgccccccacttggagctggaccctgggagcgg

SEQ ID NO: 621 - DNA, 51 bp, Homo sapiens
  allele (26)...(0): single nucleotide polymorphism
gtcggcttcttcaagcggaaccggcacacccctggaagaagatgatgaaga

SEQ ID NO: 622 - DNA, 50 bp, Homo sapiens
  allele (26)...(0): single nucleotide polymorphism
gtccatcacttcacttcagttattccctaggaggttgtatagtcttctga

SEQ ID NO: 623 - DNA, 51 bp, Homo sapiens
  allele (26)...(0): single nucleotide polymorphism
ctccagggatagttggacagaaggggagaccctggctacccaggaccagct

SEQ ID NO: 624 - DNA, 51 bp, Homo sapiens
  allele (26)...(0): single nucleotide polymorphism
gctatggaggcaaaatgggaggaaggaaacgactacagaaatgatcagcgc

SEQ ID NO: 625 - DNA, 51 bp, Homo sapiens
  allele (26)...(0): single nucleotide polymorphism
cggttactccagttatggacaaagtctattcacagtcctatggtggttatg

SEQ ID NO: 626 - DNA, 51 bp, Homo sapiens
  allele (26)...(0): single nucleotide polymorphism
ggccgagcagctgcggcgccagctggaccccctacgcacagcgcatggaga

SEQ ID NO: 627 - DNA, 51 bp, Homo sapiens
  allele (26)...(0): single nucleotide polymorphism
cagacttccacagagtgctggatgaacgcggcctgccttgccccagggtta

SEQ ID NO: 628 - DNA, 51 bp, Homo sapiens
  allele (26)...(0): single nucleotide polymorphism
tgaccacggggtgctggatgcctgccttatacatcctggaccggcggggga

SEQ ID NO: 629 - DNA, 51 bp, Homo sapiens
  allele (26)...(0): single nucleotide polymorphism
gcgccgcgagacaagggcagcggacgcgcctgcggacttgagggacagtga

SEQ ID NO: 630 - DNA, 50 bp, Homo sapiens
  allele (26)...(0): single nucleotide polymorphism
ataagttacaatgctttttttgtttaaaaaaaaaaaaagtctgtacttta

SEQ ID NO: 631 - DNA, 51 bp, Homo sapiens
  allele (26)...(0): single nucleotide polymorphism
ctgtggggctggttctgtatctgatcatcattcgattacgaaataaaacgt

SEQ ID NO: 632 - DNA, 51 bp, Homo sapiens
  allele (26)...(0): single nucleotide polymorphism
gtcagccgctacctcgactggatcctatgggcacatcagagacaaggaagc

SEQ ID NO: 633 - DNA, 51 bp, Homo sapiens
  allele (26)...(0): single nucleotide polymorphism
ccgggcagagctgcgtctgctgagggctcaagttaaaagtggagcagcacg

SEQ ID NO: 634 - DNA, 51 bp, Homo sapiens; identical to SEQ ID NO: 633
  allele (26)...(0): single nucleotide polymorphism

SEQ ID NO: 635 - DNA, 51 bp, Homo sapiens
  allele (26)...(0): single nucleotide polymorphism
aatctccgcactgcaggccaggggcctggccagctacagagagaggtcaca

SEQ ID NO: 636 - DNA, 51 bp, Homo sapiens
  allele (26)...(0): single nucleotide polymorphism
ccaggatccattttgaggattatggtgtgctgggacaccatcaactcctca

SEQ ID NO: 637 - DNA, 51 bp, Homo sapiens
  allele (26)...(0): single nucleotide polymorphism
caaatccccccgtttcttcatcttggacatgctaaaatgaaattacgcagt

SEQ ID NO: 638 - DNA, 51 bp, Homo sapiens
  allele (26)...(0): single nucleotide polymorphism
ttcttcatcttgacatgctaaaatggaaattacgcagtttctctctatcaa

SEQ ID NO: 639 - DNA, 51 bp, Homo sapiens
  allele (26)...(0): single nucleotide polymorphism
ccgcctctgctgctgctgctgctgcggcgtcccgcccagccgcagcttccc

SEQ ID NO: 640 - DNA, 50 bp, Homo sapiens
  allele (26)...(0): single nucleotide polymorphism
atccaggctgagctggatcatctgaggcctccagccacccgttttccctt

SEQ ID NO: 641 - DNA, 50 bp, Homo sapiens
  allele (26)...(0): single nucleotide polymorphism
ccaggctgagctggatcatctgaggcctccagccacccgttttcccttga

SEQ ID NO: 642 - DNA, 51 bp, Homo sapiens
  allele (26)...(0): single nucleotide polymorphism
agcgagtcctccgggaggcccacaggttactgcctccagctgcagcagtga

SEQ ID NO: 643 - DNA, 51 bp, Homo sapiens
  allele (26)...(0): single nucleotide polymorphism
cgttccagaggagcatatctgctgactgatgacctgcaagagtcatccaga

SEQ ID NO: 644 - DNA, 50 bp, Homo sapiens
  allele (26)...(0): single nucleotide polymorphism
ggaactcgagcacgtcgtcgggggacccaagatcaccggcgccctctggt

SEQ ID NO: 645 - DNA, 50 bp, Homo sapiens
  allele (26)...(0): single nucleotide polymorphism
attcccgggggagggggccctgtaaggaaaccagacaatcccatgagact

SEQ ID NO: 646 - DNA, 50 bp, Homo sapiens
  allele (26)...(0): single nucleotide polymorphism
tcccgggggagggggccctgtaaggaaaccagacaatcccatgagactcc

SEQ ID NO: 647 - DNA, 50 bp, Homo sapiens
  allele (26)...(0): single nucleotide polymorphism
gggcctgtctgcccagtggaggaggttccgctggtgttctagggggcatc

SEQ ID NO: 648 - DNA, 51 bp, Homo sapiens
  allele (26)...(0): single nucleotide polymorphism
ctctcggcactggtgactggcgagagcctggagcggcttcggagagggcta

SEQ ID NO: 649 - DNA, 50 bp, Homo sapiens
  allele (26)...(0): single nucleotide polymorphism
tcgtggccaggtccttctgcgtaagcccttgctctgccgaccttgctgga

SEQ ID NO: 650 - DNA, 50 bp, Homo sapiens
  allele (26)...(0): single nucleotide polymorphism
ggtccaaatgcaagtgctcccggaaggacccaagatccgctacagcgacg

SEQ ID NO: 651 - DNA, 50 bp, Homo sapiens
  allele (26)...(0): single nucleotide polymorphism
tccaaatgcaagtgctcccggaaggacccaagatccgctacagcgacgtg

SEQ ID NO: 652 - protein, 14 aa, Homo sapiens
  VARIANT (7)...(0): cSNP translation
PAEKDAVPQTFSVL

SEQ ID NO: 653 - protein, 14 aa, Homo sapiens
  VARIANT (7)...(0): cSNP translation
RLHRLRGEQMASYF

SEQ ID NO: 654 - protein, 14 aa, Homo sapiens
  VARIANT (7)...(0): cSNP translation
GAPLYMDSRADRKL

SEQ ID NO: 655 - protein, 14 aa, Homo sapiens
  VARIANT (7)...(0): cSNP translation
QAGTTLDLDLGGKH

SEQ ID NO: 656 - protein, 14 aa, Homo sapiens
  VARIANT (7)...(0): cSNP translation
KCECSRAYQMDLAT

SEQ ID NO: 657 - protein, 14 aa, Homo sapiens
  VARIANT (7)...(0): cSNP translation
DVRGNLKGNTEGLQ

SEQ ID NO: 658 - protein, 14 aa, Homo sapiens
  VARIANT (7)...(0): cSNP translation
VPLETPRVHSRAPS

SEQ ID NO: 659 - protein, 14 aa, Homo sapiens
  VARIANT (7)...(0): cSNP translation
PFGVIVRRQLDGRV

SEQ ID NO: 660 - protein, 14 aa, Homo sapiens
  VARIANT (7)...(0): cSNP translation
WGKEPKLESAWMNG

SEQ ID NO: 661 - protein, 14 aa, Homo sapiens
  VARIANT (7)...(0): cSNP translation
SRGYRNRRSSRETR

SEQ ID NO: 662 - protein, 14 aa, Homo sapiens
  VARIANT (7)...(0): cSNP translation
GAMLLNISGHVKES

SEQ ID NO: 663 - protein, 14 aa, Homo sapiens
  VARIANT (7)...(0): cSNP translation
QGGKLSVVLRAEDI

SEQ ID NO: 664 - protein, 14 aa, Homo sapiens
  VARIANT (7)...(0): cSNP translation
PGDISRLLEFTKAH

SEQ ID NO: 665 - protein, 14 aa, Homo sapiens
  VARIANT (7)...(0): cSNP translation
VVIPSDFFQIVGGS

SEQ ID NO: 666 - protein, 14 aa, Homo sapiens
  VARIANT (7)...(0): cSNP translation
KRKLFIRSMGEGTI

SEQ ID NO: 667 - protein, 14 aa, Homo sapiens
  VARIANT (7)...(0): cSNP translation
EQARQGLKGLEETV

SEQ ID NO: 668 - protein, 1 aa, Homo sapiens
  VARIANT (7)...(0): cSNP translation
C

SEQ ID NO: 669 - protein, 14 aa, Homo sapiens
  VARIANT (7)...(0): cSNP translation
SFLISPLTPAHAGT

SEQ ID NO: 670 - protein, 14 aa, Homo sapiens
  VARIANT (7)...(0): cSNP translation
WCSKKKDAAVMNQE

SEQ ID NO: 671 - protein, 14 aa, Homo sapiens
  VARIANT (7)...(0): cSNP translation
VALPQPLGVPNESQ

SEQ ID NO: 672 - protein, 14 aa, Homo sapiens
  VARIANT (7)...(0): cSNP translation
GIQNKEVEVRIFHC

SEQ ID NO: 673 - protein, 14 aa, Homo sapiens
  VARIANT (7)...(0): cSNP translation
VDNIRSVFGNAVSR

SEQ ID NO: 674 - protein, 14 aa, Homo sapiens
  VARIANT (7)...(0): cSNP translation
LTIQLIENHFVDEY

SEQ ID NO: 675 - protein, 14 aa, Homo sapiens
  VARIANT (7)...(0): cSNP translation
YGIPFIQTSAKTRQ

SEQ ID NO: 676 - protein, 14 aa, Homo sapiens
  VARIANT (7)...(0): cSNP translation
SASKQAVRPVLATT

SEQ ID NO: 677 - protein, 14 aa, Homo sapiens
  VARIANT (7)...(0): cSNP translation
YCMVFLALYVQARL

SEQ ID NO: 678 - protein, 14 aa, Homo sapiens
  VARIANT (7)...(0): cSNP translation
VGTYRCVPGKKGGY

SEQ ID NO: 679 - protein, 14 aa, Homo sapiens
  VARIANT (7)...(0): cSNP translation
GRATSGSEHQFCGG

SEQ ID NO: 680 - protein, 14 aa, Homo sapiens
  VARIANT (7)...(0): cSNP translation
GEWITVDQTTTANR

SEQ ID NO: 681 - protein, 14 aa, Homo sapiens
  VARIANT (7)...(0): cSNP translation
PELVLELPIRHPKF

SEQ ID NO: 682 - protein, 14 aa, Homo sapiens
  VARIANT (7)...(0): cSNP translation
EWFKDLALKWYGLP

SEQ ID NO: 683 - protein, 14 aa, Homo sapiens
  VARIANT (7)...(0): cSNP translation
YITGDRGYMDKDGY

SEQ ID NO: 684 - protein, 14 aa, Homo sapiens
  VARIANT (7)...(0): cSNP translation
SGYPKMSAHTHSSF

SEQ ID NO: 685 - protein, 14 aa, Homo sapiens
  VARIANT (7)...(0): cSNP translation
VVKAFVILASQFLS

SEQ ID NO: 686 - protein, 14 aa, Homo sapiens
  VARIANT (7)...(0): cSNP translation
DPLIYAFRSQEMRK

SEQ ID NO: 687 - protein, 14 aa, Homo sapiens
  VARIANT (7)...(0): cSNP translation
LATLPEYVVYKPQM

SEQ ID NO: 688 - protein, 14 aa, Homo sapiens
  VARIANT (7)...(0): cSNP translation
LAPQQRVAPQQKRS

SEQ ID NO: 689 - protein, 14 aa, Homo sapiens
  VARIANT (7)...(0): cSNP translation
LYRDIFEHLRDESG

SEQ ID NO: 690 - protein, 14 aa, Homo sapiens
  VARIANT (7)...(0): cSNP translation
DFYYLGAFFGGSVQ

SEQ ID NO: 691 - protein, 14 aa, Homo sapiens
  VARIANT (7)...(0): cSNP translation
LTMVTLVTLPLLFL

SEQ ID NO: 692 - protein, 14 aa, Homo sapiens
  VARIANT (7)...(0): cSNP translation
RALYLLIRRVLHLG

SEQ ID NO: 693 - protein, 14 aa, Homo sapiens
  VARIANT (7)...(0): cSNP translation
EEAMNAVYSGYVYT

SEQ ID NO: 694 - protein, 14 aa, Homo sapiens
  VARIANT (7)...(0): cSNP translation
SAASYNVKTAYRKY

SEQ ID NO: 695 - protein, 8 aa, Homo sapiens
  VARIANT (7)...(0): cSNP translation
ESTTVGSS

SEQ ID NO: 696 - protein, 14 aa, Homo sapiens
  VARIANT (7)...(0): cSNP translation
QQWSEHHAFLSQGS

SEQ ID NO: 697 - protein, 14 aa, Homo sapiens
  VARIANT (7)...(0): cSNP translation
DNFSVTEVPFTESA

SEQ ID NO: 698 - protein, 14 aa, Homo sapiens
  VARIANT (7)...(0): cSNP translation
PGRRQRLTMAIRTV

SEQ ID NO: 699 - protein, 14 aa, Homo sapiens
  VARIANT (7)...(0): cSNP translation
LAGKVAQVKKNGRI

SEQ ID NO: 700 - protein, 14 aa, Homo sapiens
  VARIANT (7)...(0): cSNP translation
LENQKKVRKKKVLI

SEQ ID NO: 701 - protein, 14 aa, Homo sapiens
  VARIANT (7)...(0): cSNP translation
VSDEELDQMLDSGQ

SEQ ID NO: 702 - protein, 14 aa, Homo sapiens
  VARIANT (7)...(0): cSNP translation
WLGFNKQRGHLQIA

SEQ ID NO: 703 - protein, 14 aa, Homo sapiens
  VARIANT (7)...(0): cSNP translation
EPECREVFHRRARA

SEQ ID NO: 704 - protein, 14 aa, Homo sapiens
  VARIANT (7)...(0): cSNP translation
LPCGPGVKGRCFGP

SEQ ID NO: 705 - protein, 14 aa, Homo sapiens
  VARIANT (7)...(0): cSNP translation
SAMDTRLLCCAVIC

SEQ ID NO: 706 - protein, 14 aa, Homo sapiens
  VARIANT (7)...(0): cSNP translation
NTRLLCHVMLCLLG

SEQ ID NO: 707 - protein, 14 aa, Homo sapiens
  VARIANT (7)...(0): cSNP translation
QTGDSAIYLCAVEA

SEQ ID NO: 708 - protein, 14 aa, Homo sapiens
  VARIANT (7)...(0): cSNP translation
VYLCAVDAYSNDYK

SEQ ID NO: 709 - protein, 14 aa, Homo sapiens
  VARIANT (7)...(0): cSNP translation
QKQMELDSILVALL

SEQ ID NO: 710 - protein, 14 aa, Homo sapiens
  VARIANT (7)...(0): cSNP translation
GSTVIAGSINAHGS

SEQ ID NO: 711 - protein, 14 aa, Homo sapiens
  VARIANT (7)...(0): cSNP translation
PVMGLMIYMMVMDH

SEQ ID NO: 712 - protein, 14 aa, Homo sapiens
  VARIANT (7)...(0): cSNP translation
SSQDPASVRECHDP

SEQ ID NO: 713 - protein, 14 aa, Homo sapiens
  VARIANT (7)...(0): cSNP translation
VLLLLGACAAPPAW

SEQ ID NO: 714 - protein, 14 aa, Homo sapiens
  VARIANT (7)...(0): cSNP translation
SLPYAVAPLSLPRG

SEQ ID NO: 715 - protein, 14 aa, Homo sapiens
  VARIANT (7)...(0): cSNP translation
YGPQCQLVIQCEPL

SEQ ID NO: 716 - protein, 14 aa, Homo sapiens
  VARIANT (7)...(0): cSNP translation
TMDCTHSLGNFSFS

SEQ ID NO: 717 - protein, 14 aa, Homo sapiens
  VARIANT (7)...(0): cSNP translation
GTTETGGQGKGTSK

SEQ ID NO: 718 - protein, 14 aa, Homo sapiens
  VARIANT (7)...(0): cSNP translation
CNGVAVCSNQDLIT

SEQ ID NO: 719 - protein, 14 aa, Homo sapiens
  VARIANT (7)...(0): cSNP translation
LVSYCPRRLQQLLP

SEQ ID NO: 720 - protein, 14 aa, Homo sapiens
  VARIANT (7)...(0): cSNP translation
RPGSPEGPLGPGGP

SEQ ID NO: 721 - protein, 14 aa, Homo sapiens
  VARIANT (7)...(0): cSNP translation
YAERYQMPTGIKGP

SEQ ID NO: 722 - protein, 14 aa, Homo sapiens
  VARIANT (7)...(0): cSNP translation
NPLVPGTPGRPGIP

SEQ ID NO: 723 - protein, 14 aa, Homo sapiens
  VARIANT (7)...(0): cSNP translation
VTNRPCGSQVRCEG

SEQ ID NO: 724 - protein, 14 aa, Homo sapiens
  VARIANT (7)...(0): cSNP translation
STTCVRQAQCGQDF

SEQ ID NO: 725 - protein, 14 aa, Homo sapiens
  VARIANT (7)...(0): cSNP translation
GGQPCTAPLVAFQP

SEQ ID NO: 726 - protein, 14 aa, Homo sapiens
  VARIANT (7)...(0): cSNP translation
SPRISSPRPQGLSN

SEQ ID NO: 727 - protein, 14 aa, Homo sapiens
  VARIANT (7)...(0): cSNP translation
PMTQTTSLKTSWVN

SEQ ID NO: 728 - protein, 12 aa, Homo sapiens
  VARIANT (7)...(0): cSNP translation
INAYISHLGFRF

SEQ ID NO: 729 - protein, 14 aa, Homo sapiens
  VARIANT (7)...(0): cSNP translation
LHSGHRQRPEFRPY

SEQ ID NO: 730 - protein, 14 aa, Homo sapiens
  VARIANT (7)...(0): cSNP translation
CSQEAKQSAYCPYS

SEQ ID NO: 731 - protein, 14 aa, Homo sapiens [sequence omitted]

SEQ ID NO: 732 - protein, 14 aa, Homo sapiens
  VARIANT (7)...(0): cSNP translation
NADIIEALRKKGFK

SEQ ID NO: 733 - protein, 9 aa, Homo sapiens
  VARIANT (7)...(0): cSNP translation
DRVENYKPR

SEQ ID NO: 734 - protein, 14 aa, Homo sapiens
  VARIANT (7)...(0): cSNP translation
IQTAELTPELVISN

SEQ ID NO: 735 - protein, 14 aa, Homo sapiens
  VARIANT (7)...(0): cSNP translation
EQQQEQHQEQQQEQ

SEQ ID NO: 736 - protein, 14 aa, Homo sapiens
  VARIANT (7)...(0): cSNP translation
LSFISTELKYPGMP

SEQ ID NO: 737 - protein, 14 aa, Homo sapiens
  VARIANT (7)...(0): cSNP translation
ILPSGLAFISTGLK

SEQ ID NO: 738 - protein, 14 aa, Homo sapiens
  VARIANT (7)...(0): cSNP translation
FYLAMNEEGKLYAK

SEQ ID NO: 739 - protein, 14 aa, Homo sapiens
  VARIANT (7)...(0): cSNP translation
GLDQKRIKYVVGEL

SEQ ID NO: 740 - protein, 14 aa, Homo sapiens
  VARIANT (7)...(0): cSNP translation
AGLGQVRLIVGILL

SEQ ID NO: 741 - protein, 14 aa, Homo sapiens
  VARIANT (7)...(0): cSNP translation
TLTSVGHQSVTPGE

SEQ ID NO: 742 - protein, 14 aa, Homo sapiens
  VARIANT (7)...(0): cSNP translation
GQKTLTPVGYQSVT

SEQ ID NO: 743 - protein, 14 aa, Homo sapiens
  VARIANT (7)...(0): cSNP translation
TEITGATTMTSVGH

SEQ ID NO: 744 - protein, 14 aa, Homo sapiens
  VARIANT (7)...(0): cSNP translation
GVYILTYNTSQYDT

SEQ ID NO: 745 - protein, 14 aa, Homo sapiens
  VARIANT (7)...(0): cSNP translation
PLSFHVIWIASFYN

SEQ ID NO: 746 - protein, 14 aa, Homo sapiens
  VARIANT (7)...(0): cSNP translation
STIVFLCPWSRGNF

SEQ ID NO: 747 - protein, 13 aa, Homo sapiens
  VARIANT (7)...(0): cSNP translation
PDPREACGSSSYV

SEQ ID NO: 748 - protein, 14 aa, Homo sapiens
  VARIANT (7)...(0): cSNP translation
AKDMNGTSLHGKAI

SEQ ID NO: 749 - protein, 14 aa, Homo sapiens
  VARIANT (7)...(0): cSNP translation
RSARGSRGGTRGWL

SEQ ID NO: 750 - protein, 14 aa, Homo sapiens
  VARIANT (7)...(0): cSNP translation
NFTSKYHMKVLYLS

SEQ ID NO: 751 - protein, 14 aa, Homo sapiens
  VARIANT (7)...(0): cSNP translation
IQYTYLGGHVCLSA

SEQ ID NO: 752 - protein, 14 aa, Homo sapiens
  VARIANT (7)...(0): cSNP translation
LDPNNPDANWIHAR

SEQ ID NO: 753 - protein, 14 aa, Homo sapiens
  VARIANT (7)...(0): cSNP translation
CLTERQSKIWFQNR

SEQ ID NO: 754 - protein, 14 aa, Homo sapiens
  VARIANT (7)...(0): cSNP translation
PYRIAHAVIKAHAR

SEQ ID NO: 755 - protein, 14 aa, Homo sapiens
  VARIANT (7)...(0): cSNP translation
KRKKEVHATSPAPS

SEQ ID NO: 756 - protein, 14 aa, Homo sapiens
  VARIANT (7)...(0): cSNP translation
IKNEARLPCLPTPG

SEQ ID NO: 757 - protein, 14 aa, Homo sapiens
  VARIANT (7)...(0): cSNP translation
ARKFFENLPDGTWN

SEQ ID NO: 758 - protein, 8 aa, Homo sapiens
  VARIANT (7)...(0): cSNP translation
FSYSASST

SEQ ID NO: 759 - protein, 14 aa, Homo sapiens
  VARIANT (7)...(0): cSNP translation
PVSSANVMSGISSV

SEQ ID NO: 760 - protein, 14 aa, Homo sapiens
  VARIANT (7)...(0): cSNP translation
WERFVHRENQHLVS

SEQ ID NO: 761 - protein, 14 aa, Homo sapiens
  VARIANT (7)...(0): cSNP translation
GTEDLYGYIDKYNI

SEQ ID NO: 762 - protein, 14 aa, Homo sapiens
  VARIANT (7)...(0): cSNP translation
KQLYQTFTDYDIRF

SEQ ID NO: 763 - protein, 14 aa, Homo sapiens
  VARIANT (7)...(0): cSNP translation
GSLHPHPPYHIRVA

SEQ ID NO: 764 - protein, 14 aa, Homo sapiens
  VARIANT (7)...(0): cSNP translation
LYSRLGGQPVYLPT

SEQ ID NO: 765 - protein, 14 aa, Homo sapiens
  VARIANT (7)...(0): cSNP translation
APPGAYHGAPGAYP

SEQ ID NO: 766 - protein, 14 aa, Homo sapiens
  VARIANT (7)...(0): cSNP translation
VMPHSSEHKTAQPN

SEQ ID NO: 767 - protein, 14 aa, Homo sapiens
  VARIANT (7)...(0): cSNP translation
TCVVEHTGAPEPIL

SEQ ID NO: 768 - protein, 14 aa, Homo sapiens
  VARIANT (7)...(0): cSNP translation
VGFLVGIVLIIMGT

SEQ ID NO: 769 - protein, 7 aa, Homo sapiens
  VARIANT (7)...(0): cSNP translation
QSVVSCA

SEQ ID NO: 770 - protein, 14 aa, Homo sapiens
  VARIANT (7)...(0): cSNP translation
PGPTVRAGENVTLS

SEQ ID NO: 771 - protein, 14 aa, Homo sapiens
  VARIANT (7)...(0): cSNP translation
CSGVWGADTEERLV

SEQ ID NO: 772 - protein, 14 aa, Homo sapiens
  VARIANT (7)...(0): cSNP translation
ALLRHELKGYQKWV

SEQ ID NO: 773 - protein, 14 aa, Homo sapiens
  VARIANT (7)...(0): cSNP translation
LLRHEWQGYQKWVR

SEQ ID NO: 774 - protein, 14 aa, Homo sapiens
  VARIANT (7)...(0): cSNP translation
GFHYGVLACEGCKG

SEQ ID NO: 775 - protein, 14 aa, Homo sapiens
  VARIANT (7)...(0): cSNP translation
DGPEQAHRQRRRQR

SEQ ID NO: 776 - protein, 1 aa, Homo sapiens
  VARIANT (7)...(0): cSNP translation
E

SEQ ID NO: 777 - protein, 14 aa, Homo sapiens
  VARIANT (7)...(0): cSNP translation
NHNGEWFEAQTKNG

SEQ ID NO: 778 - protein, 14 aa, Homo sapiens
  VARIANT (7)...(0): cSNP translation
LVVVGACGVGKSAL

SEQ ID NO: 779 - protein, 14 aa, Homo sapiens
  VARIANT (7)...(0): cSNP translation
ILDTAGHEEYSAMR

SEQ ID NO: 780 - protein, 14 aa, Homo sapiens
  VARIANT (7)...(0): cSNP translation
FGHQENSQNEEILN

SEQ ID NO: 781 - protein, 14 aa, Homo sapiens
  VARIANT (7)...(0): cSNP translation
LTYPPGPRTQLRED

SEQ ID NO: 782 - protein, 14 aa, Homo sapiens
  VARIANT (7)...(0): cSNP translation
QPQARQEEQVRVVR

SEQ ID NO: 783 - protein, 14 aa, Homo sapiens
  VARIANT (7)...(0): cSNP translation
NAVKAETRQQFRSL

SEQ ID NO: 784 - protein, 14 aa, Homo sapiens
  VARIANT (7)...(0): cSNP translation
AQIFDYSEIPNFPR

SEQ ID NO: 785 - protein, 14 aa, Homo sapiens
  VARIANT (7)...(0): cSNP translation
DDDDAPRPSQFEED

SEQ ID NO: 786 - protein, 14 aa, Homo sapiens
  VARIANT (7)...(0): cSNP translation
HVLRMHGYRAPGEQ

SEQ ID NO: 787 - protein, 14 aa, Homo sapiens
  VARIANT (7)...(0): cSNP translation
ETQLGTLAQFPNTL

SEQ ID NO: 788 - protein, 14 aa, Homo sapiens
  VARIANT (7)...(0): cSNP translation
EAMERFGEDEGFIK

SEQ ID NO: 789 - protein, 14 aa, Homo sapiens
  VARIANT (7)...(0): cSNP translation
EEEKPLARNEFQRQ

SEQ ID NO: 790 - protein, 14 aa, Homo sapiens
  VARIANT (7)...(0): cSNP translation
DAHFDEHERWTNNF

SEQ ID NO: 791 - protein, 14 aa, Homo sapiens
  VARIANT (7)...(0): cSNP translation
RWTNNFTEYNLHRV

SEQ ID NO: 792 - protein, 14 aa, Homo sapiens
  VARIANT (7)...(0): cSNP translation
DSGLLQCKLALLSV

SEQ ID NO: 793 - protein, 14 aa, Homo sapiens
  VARIANT (7)...(0): cSNP translation
EAERVKEQTFREKA

SEQ ID NO: 794 - protein, 14 aa, Homo sapiens
  VARIANT (7)...(0): cSNP translation
AFVVLALQFLSHDP

SEQ ID NO: 795 - protein, 14 aa, Homo sapiens
  VARIANT (7)...(0): cSNP translation
TGKIQRTKLRDKEW

SEQ ID NO: 796 - protein, 14 aa, Homo sapiens
  VARIANT (7)...(0): cSNP translation
VDYMTQARGQRSSL

SEQ ID NO: 797 - protein, 14 aa, Homo sapiens
  VARIANT (7)...(0): cSNP translation
NNLLYITPEAFQNL

SEQ ID NO: 798 - protein, 14 aa, Homo sapiens
  VARIANT (7)...(0): cSNP translation
LRIQCLCRKQSSKH

SEQ ID NO: 799 - protein, 14 aa, Homo sapiens
  VARIANT (7)...(0): cSNP translation
LEKIQPMTQNGQHP

SEQ ID NO: 800 - protein, 14 aa, Homo sapiens
  VARIANT (7)...(0): cSNP translation
WMIFVVIASVFTNG

SEQ ID NO: 801 - protein, 14 aa, Homo sapiens
  VARIANT (7)...(0): cSNP translation
SINLFSGIFFLTCM

SEQ ID NO: 802 - protein, 14 aa, Homo sapiens
  VARIANT (7)...(0): cSNP translation
VDICFSSTTVPKML

SEQ ID NO: 803 - protein, 14 aa, Homo sapiens
  VARIANT (7)...(0): cSNP translation
ESNTTGTTAFSMPS

SEQ ID NO: 804 - protein, 14 aa, Homo sapiens
  VARIANT (7)...(0): cSNP translation
VHPVRPLRLESFSA

SEQ ID NO: 805 - protein, 14 aa, Homo sapiens
  VARIANT (7)...(0): cSNP translation
RGARPGPRVPKTLV

SEQ ID NO: 806 - protein, 14 aa, Homo sapiens
  VARIANT (7)...(0): cSNP translation
KAFLTSHSLRIHVR

SEQ ID NO: 807 - protein, 14 aa, Homo sapiens
  VARIANT (7)...(0): cSNP translation
SEASSAFFMAKKKT

SEQ ID NO: 808 - protein, 14 aa, Homo sapiens
  VARIANT (7)...(0): cSNP translation
ELYRDILQHLRDES

SEQ ID NO: 809 - protein, 14 aa, Homo sapiens
  VARIANT (7)...(0): cSNP translation
YVFTDQLAAVPRVT

SEQ ID NO: 810 - protein, 14 aa, Homo sapiens
  VARIANT (7)...(0): cSNP translation
LSVLEVGAYKRWQD

SEQ ID NO: 811 - protein, 14 aa, Homo sapiens
  VARIANT (7)...(0): cSNP translation
VDVDMEIRDHVGVE

SEQ ID NO: 812 - protein, 14 aa, Homo sapiens
  VARIANT (7)...(0): cSNP translation
FGTLHPSFYGSSRE

SEQ ID NO: 813 - protein, 14 aa, Homo sapiens
  VARIANT (7)...(0): cSNP translation
EGDFYYMGGFFGGS

SEQ ID NO: 814 - protein, 14 aa, Homo sapiens
  VARIANT (7)...(0): cSNP translation
GGSVQEMQRLTRAC

SEQ ID NO: 815 - protein, 14 aa, Homo sapiens
  VARIANT (7)...(0): cSNP translation
VVELTQDDALGSRW

SEQ ID NO: 816 - protein, 14 aa, Homo sapiens
  VARIANT (7)...(0): cSNP translation
KDFHKDMLKPSPGK

SEQ ID NO: 817 - protein, 14 aa, Homo sapiens
  VARIANT (7)...(0): cSNP translation
TNNCYRHAIVTTSI

SEQ ID NO: 818 - protein, 14 aa, Homo sapiens
  VARIANT (7)...(0): cSNP translation
GFVVFSSLGYMAQK

SEQ ID NO: 819 - protein, 14 aa, Homo sapiens
  VARIANT (7)...(0): cSNP translation
MDEAARPEAWDSYR

SEQ ID NO: 820 - protein, 14 aa, Homo sapiens
  VARIANT (7)...(0): cSNP translation
SPQSSARGKPAMSY

SEQ ID NO: 821 - protein, 14 aa, Homo sapiens
  VARIANT (7)...(0): cSNP translation
LEDLAGWKELFQTP

SEQ ID NO: 822 - protein, 14 aa, Homo sapiens
  VARIANT (7)...(0): cSNP translation
VQDILRLEMPASKI

SEQ ID NO: 823 - protein, 14 aa, Homo sapiens
  VARIANT (7)...(0): cSNP translation
SERETEHTPALIMV

SEQ ID NO: 824 - protein, 14 aa, Homo sapiens
  VARIANT (7)...(0): cSNP translation
KVLDHWCIMTSEEE

SEQ ID NO: 825 - protein, 14 aa, Homo sapiens
  VARIANT (7)...(0): cSNP translation
QEVHGPYPDSSFLT

SEQ ID NO: 826 - protein, 14 aa, Homo sapiens
  VARIANT (7)...(0): cSNP translation
QEVHGPIPDSSFLT

SEQ ID NO: 827 - protein, 14 aa, Homo sapiens
  VARIANT (7)...(0): cSNP translation
ELCHEKGILEKYGH

SEQ ID NO: 828 - protein, 14 aa, Homo sapiens
  VARIANT (7)...(0): cSNP translation
NNNLRHTDEMFWNH

SEQ ID NO: 829 - protein, 14 aa, Homo sapiens
  VARIANT (7)...(0): cSNP translation
GMASSCSVQVKLEL

SEQ ID NO: 830 - protein, 14 aa, Homo sapiens
  VARIANT (7)...(0): cSNP translation
PSIFIYRHTASGKT

SEQ ID NO: 831 - protein, 14 aa, Homo sapiens
  VARIANT (7)...(0): cSNP translation
PPPPPGAPGGSQDT

SEQ ID NO: 832 - protein, 14 aa, Homo sapiens
  VARIANT (7)...(0): cSNP translation
RAALERGKAIEKNL

SEQ ID NO: 833 - protein, 14 aa, Homo sapiens
  VARIANT (7)...(0): cSNP translation
TGGLLLRLALMLQL

SEQ ID NO: 834 - protein, 14 aa, Homo sapiens
  VARIANT (7)...(0): cSNP translation
DSSSSNGKAKNPPG

SEQ ID NO: 835 - protein, 14 aa, Homo sapiens
  VARIANT (7)...(0): cSNP translation
LEPQWYSVLEKDSV

SEQ ID NO: 836 - protein, 14 aa, Homo sapiens
  VARIANT (7)...(0): cSNP translation
QKHSSGXSNTSTAN

SEQ ID NO: 837 - protein, 14 aa, Homo sapiens
  VARIANT (7)...(0): cSNP translation
WGTEDDATQSYHNG

SEQ ID NO: 838 - protein, 14 aa, Homo sapiens
  VARIANT (7)...(0): cSNP translation
RCMGTVNLNQARGS

SEQ ID NO: 839 - protein, 14 aa, Homo sapiens
  VARIANT (7)...(0): cSNP translation
FSKLLGPLSAKKYL

SEQ ID NO: 840 - protein, 14 aa, Homo sapiens
  VARIANT (7)...(0): cSNP translation
TSKILFFSQGSEIA

SEQ ID NO: 841 - protein, 14 aa, Homo sapiens
  VARIANT (7)...(0): cSNP translation
YVQPPEVIGPMRPE

SEQ ID NO: 842 - protein, 14 aa, Homo sapiens
  VARIANT (7)...(0): cSNP translation
GPDGQEVDPPNPEE

SEQ ID NO: 843 - protein, 14 aa, Homo sapiens
  VARIANT (7)...(0): cSNP translation
KGGEQKRHEKISAS

SEQ ID NO: 844 - protein, 14 aa, Homo sapiens
  VARIANT (7)...(0): cSNP translation
APQQEGEASKEKEE

SEQ ID NO: 845 - protein, 14 aa, Homo sapiens
  VARIANT (7)...(0): cSNP translation
KVVQSPQSLVVHEG

SEQ ID NO: 846 - protein, 14 aa, Homo sapiens
  VARIANT (7)...(0): cSNP translation
LNCSYEMTNFRSLL

SEQ ID NO: 847 - protein, 14 aa, Homo sapiens
  VARIANT (7)...(0): cSNP translation
TNFRSLQWYKQEKK

SEQ ID NO: 848 - protein, 14 aa, Homo sapiens
  VARIANT (7)...(0): cSNP translation
LDKKELSSILNITA

SEQ ID NO: 849 - protein, 14 aa, Homo sapiens
  VARIANT (8)...(0): cSNP translation
EYVVGAPHLELDPG

SEQ ID NO: 850 - protein, 12 aa, Homo sapiens
  VARIANT (7)...(0): cSNP translation
FFKRNRHTPGRR

SEQ ID NO: 851 - protein, 7 aa, Homo sapiens
  VARIANT (7)...(0): cSNP translation
TIQPPRE

SEQ ID NO: 852 - protein, 14 aa, Homo sapiens
  VARIANT (8)...(0): cSNP translation
GIVGQKGRPWLPRT

SEQ ID NO: 853 - protein, 12 aa, Homo sapiens
  VARIANT (8)...(0): cSNP translation
GGKMGGRKRLQK

SEQ ID NO: 854 - protein, 14 aa, Homo sapiens
  VARIANT (8)...(0): cSNP translation
YSSYGQSLFTVLWW

SEQ ID NO: 855 - protein, 14 aa, Homo sapiens
  VARIANT (8)...(0): cSNP translation
EQLRRQLDPLRTAH

SEQ ID NO: 856 - protein, 14 aa, Homo sapiens
  VARIANT (7)...(0): cSNP translation
STECWMNAACLAPG

SEQ ID NO: 857 - protein, 14 aa, Homo sapiens
  VARIANT (9)...(0): cSNP translation
HGVLDACLIHPGPA

SEQ ID NO: 858 - protein, 14 aa, Homo sapiens
  VARIANT (7)...(0): cSNP translation
RDKGSGRACGLEGQ

SEQ ID NO: 859 - protein, 6 aa, Homo sapiens
  VARIANT (7)...(0): cSNP translation
TDFFFF

SEQ ID NO: 860 - protein, 13 aa, Homo sapiens
  VARIANT (8)...(0): cSNP translation
GAGSVSDHHSITK

SEQ ID NO: 861 - protein, 12 aa, Homo sapiens
  VARIANT (7)...(0): cSNP translation
RYLDWILWAHQR

SEQ ID NO: 862 - protein, 14 aa, Homo sapiens
  VARIANT (8)...(0): cSNP translation
AELRLLRAQVKSGA

SEQ ID NO: 863 - protein, 14 aa, Homo sapiens
  VARIANT (8)...(0): cSNP translation
AELRLLRAQVKSGA

SEQ ID NO: 864 - protein, 14 aa, Homo sapiens
  VARIANT (8)...(0): cSNP translation
PHCRPGAWPATERG

SEQ ID NO: 865 - protein, 14 aa, Homo sapiens
  VARIANT (8)...(0): cSNP translation
IHFEDYGVLGHHQL

SEQ ID NO: 866 - protein, 8 aa, Homo sapiens
  VARIANT (7)...(0): cSNP translation
NFILACPR

SEQ ID NO: 867 - protein, 13 aa, Homo sapiens
  VARIANT (7)...(0): cSNP translation
REKLRNFHFSMSR

SEQ ID NO: 868 - protein, 14 aa, Homo sapiens
  VARIANT (8)...(0): cSNP translation
LLLLLLRRPAQPQL

SEQ ID NO: 869 - protein, 8 aa, Homo sapiens
  VARIANT (7)...(0): cSNP translation
KRVAGGLR

SEQ ID NO: 870 - protein, 9 aa, Homo sapiens
  VARIANT (8)...(0): cSNP translation
GKRVAGGLR

SEQ ID NO: 871 - protein, 14 aa, Homo sapiens
  VARIANT (7)...(0): cSNP translation
SSGRPTGYCLQLQQ

SEQ ID NO: 872 - protein, 7 aa, Homo sapiens
  VARIANT (8)...(0): cSNP translation
QRSISAD

SEQ ID NO: 873 - protein, 14 aa, Homo sapiens
  VARIANT (8)...(0): cSNP translation
RAPVILGPPTTCSS

SEQ ID NO: 874 - protein, 14 aa, Homo sapiens
  VARIANT (7)...(0): cSNP translation
MGLSGFLTGPPPPG

SEQ ID NO: 875 - protein, 14 aa, Homo sapiens
  VARIANT (8)...(0): cSNP translation
LMGLSGFLTGPPPP

SEQ ID NO: 876 - protein, 14 aa, Homo sapiens
  VARIANT (7)...(0): cSNP translation
PRTPAEPPPLGRQA

SEQ ID NO: 877 - protein, 14 aa, Homo sapiens
  VARIANT (7)...(0): cSNP translation
GTGDWREPGAASER

SEQ ID NO: 878 - protein, 14 aa, Homo sapiens
  VARIANT (9)...(0): cSNP translation
QGRQSKGLRRRTWP

SEQ ID NO: 879 - protein, 14 aa, Homo sapiens
  VARIANT (8)...(0): cSNP translation
KCKCSRKDPRSATA

SEQ ID NO: 880 - protein, 14 aa, Homo sapiens
  VARIANT (7)...(0): cSNP translation
CKCSRKDPRSATAT

(19) United States
(12) Patent Application Publication
(10) Pub. No.: US 2004/0235041 A1
(43) Pub. Date: Nov. 25, 2004

(54) Nucleic acids containing single nucleotide polymorphisms and methods of use thereof

(76) Inventors: Richard A. Shimkets, West Haven, CT (US); Martin Leach, Webster, MA (US)

Correspondence Address: Jenell Lawson, CuraGen Corporation, Intellectual Property, 555 Long Wharf Drive, New Haven, CT, 06511, US

(21) Appl. No.: 10/865,478

(22) Filed: Jun. 10, 2004

Related U.S. Application Data
(63) Continuation of application No. 09/472,688, filed on Dec. 27, 1999.
Application No. 09/472,688 is a continuation-in-part of application No. 09/442,849, filed on Nov. 17, 1999, now abandoned.
(60) Provisional application No. 60/109,024, filed on Nov. 17, 1998.

(51) Int. Cl. C12Q 1/68; C07H 21/04

(52) U.S. Cl. 435/6; 536/23.2

(57) ABSTRACT

The invention provides nucleic acids containing single-nucleotide polymorphisms identified for transcribed human sequences, as well as methods of using the nucleic acids.

### RELATED APPLICATIONS

This application is a continuation-in-part of U.S. Ser. No. 09/442,849, filed Nov. 17, 1999, which claims priority to U.S. Ser. No. 09/442,129 and U.S. Ser. No. ______, both filed Nov. 16, 1999, all of which are entitled “Nucleic Acids Containing Single Nucleotide Polymorphisms and Methods of Use Thereof” and naming Richard Shimkets and Martin Leach as inventors, and to U.S. Ser. No. 60/109,024, filed Nov. 17, 1998. The contents of these applications are incorporated herein by reference in their entirety.

### FIELD OF THE INVENTION

The invention relates generally to nucleic acids and polypeptides and in particular to the identification of human single nucleotide polymorphisms based on at least one gene product that was not previously described.

### BACKGROUND OF THE INVENTION

Sequence polymorphism-based analysis of nucleic acid is generally based on alterations in nucleic acid sequences between related individuals. This analysis has been widely used in a variety of genetic, diagnostic, and forensic applications. For example, polymorphism analyses are used in identity and paternity analysis, and in genetic mapping studies.

Several different types of polymorphisms in nucleic acid have been described. One such type of variation is a restriction fragment length polymorphism (RFLP). RFLPS can create or delete a recognition sequence for a restriction endonuclease in one nucleic acid relative to a second nucleic acid. The result of the variation is in an alteration the relative length of restriction enzyme generated DNA fragments in the two nucleic acids.

Other polymorphisms take the form of short tandem repeats (STR) sequences, which are also referred to as variable numbers of tandem repeat (VNTR) sequences. STR sequences typically that include tandem repeats of 2, 3, or 4 nucleotide sequences that are present in a nucleic acid from one individual but absent from a second, related individual at the corresponding genomic location.

Other polymorphisms take the form of single nucleotide variations, termed single nucleotide polymorphisms (SNPs), between individuals. A SNP can, in some instances, be referred to as a “cSNP” to denote that the nucleotide sequence containing the SNP originates as a cDNA.

SNPs can arise in several ways. A single nucleotide polymorphism may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or the converse.

Single nucleotide polymorphisms can also arise from a deletion of a nucleotide or an insertion of a nucleotide relative to a reference allele. Thus, the polymorphic site is a site at which one allele bears a gap with respect to a single nucleotide in another allele. Some SNPs occur within, or near genes. One such class includes SNPs falling within regions of genes encoding for a polypeptide product. These SNPs may result in an alteration of the amino acid sequence of the polypeptide product and give rise to the expression of a defective or other variant protein. Such variant products can, in some cases result in a pathological condition, e.g., genetic disease. Examples of genes in which a polymorphism within a coding sequence gives rise to genetic disease include sickle cell anemia and cystic fibrosis. Other SNPs do not result in alteration of the polypeptide product. Of course, SNPs can also occur in noncoding regions of genes.

SNPs tend to occur with great frequency and are spaced uniformly throughout the genome. The frequency and uniformity of SNPs means that there is a greater probability that such a polymorphism will be found in close proximity to a genetic locus of interest.

### SUMMARY OF THE INVENTION

The invention is based in part on the discovery of single nucleotide polymorphisms (SNPs) in regions of human DNA.

Accordingly, in one aspect, the invention provides nucleic acid sequences comprising nucleic acid segments of both publicly known and novel genes, including the polymorphic site. The segments can be DNA or RNA, and can be single- or double-stranded. Preferred segments include a biallelic polymorphic site.

The invention further provides allele-specific oligonucleotides that hybridize to a segment of a fragment shown in Table 1, column 4, or its complement. These oligonucleotides can be probes or primers. Also provided are isolated nucleic acids comprising a sequence shown in Table 1, column 4, in which the polymorphic site within the sequence is occupied by a base other than the reference bases shown in Table 1, columns 5 and 6.

The invention further provides a method of analyzing a nucleic acid from an individual. The method determines which base is present at any one of the polymorphic sites shown in Table 1. Optionally, a set of bases occupying a set of polymorphic sites shown in Table 1 is determined. This type of analysis can be performed on a number of individuals, who are tested for the presence of a disease phenotype.

In another aspect, the invention provides an isolated polynucleotide which includes one or more of the SNPs described herein. The polynucleotide can be, e.g., a nucleotide sequence which includes one or more of the polymorphic sequences shown in Table 1 and which includes a polymorphic sequence, or a fragment of the polymorphic sequence, as long as it includes the polymorphic site. The polynucleotide may alternatively contain a nucleotide sequence which includes a sequence complementary to one or more of these sequences, or a fragment of the complementary nucleotide sequence, provided that the fragment includes a polymorphic site in the polymorphic sequence.

The polynucleotide can be, e.g., DNA or RNA, and can be between about 10 and about 100 nucleotides, e.g, 10-90, 10-75, 10-51, 10-40, or 10-30, nucleotides in length.

In preferred embodiments, the polymorphic site in the polymorphic sequence includes a nucleotide other than the nucleotide listed in Table 1, column 5 for the polymorphic sequence, e.g., the polymorphic site includes the nucleotide listed in Table 1, column 6 for the polymorphic sequence.

In other embodiments, the complement of the polymorphic site includes a nucleotide other than the complement of the nucleotide listed in Table 1, column 5 for the complement of the polymorphic sequence, e.g., the complement of the nucleotide listed in Table 1, column 6 for the polymorphic sequence.

In some embodiments, the polymorphic sequence is associated with a polypeptide related to one of the protein families disclosed herein. For example, the nucleic acid may be associated with a polypeptide related to angiopoietin, 4-hydroxybutyrate dehydrogenase, or any of the other proteins identified in Table 1, column 10.

In another aspect, the invention provides an isolated allele-specific oligonucleotide that hybridizes to a first polynucleotide containing a polymorphic site. The first polynucleotide can be, e.g., a nucleotide sequence comprising one or more polymorphic sequences recited in Table 1, provided that the polymorphic sequence includes a nucleotide other than the nucleotide recited in Table 1, column 5 for the polymorphic sequence. Alternatively, the first polynucleotide can be a nucleotide sequence that is a fragment of the polymorphic sequence, provided that the fragment includes a polymorphic site in the polymorphic sequence, or a complementary nucleotide sequence which includes a sequence complementary to one or more polymorphic sequences in Table 1, provided that the complementary nucleotide sequence includes a nucleotide other than the complement of the nucleotide recited in Table 1, column 5. The first polynucleotide may in addition include a nucleotide sequence that is a fragment of the complementary sequence, provided that the fragment includes a polymorphic site in the polymorphic sequence.

In some embodiments, the oligonucleotide does not hybridize under stringent conditions to a second polynucleotide. The second polynucleotide can be, e.g., (a) a nucleotide sequence comprising one or more polymorphic sequences in Table 1, wherein the polymorphic sequence includes the nucleotide listed in Table 1, column 5 for the polymorphic sequence; (b) a nucleotide sequence that is a fragment of any of the polymorphic sequences; (c) a complementary nucleotide sequence including a sequence complementary to one or more polymorphic sequences disclosed herein in Table 1; and (d) a nucleotide sequence that is a fragment of the complementary sequence, provided that the fragment includes a polymorphic site in the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

The invention also provides a method of detecting a polymorphic site in a nucleic acid. The method includes contacting the nucleic acid with an oligonucleotide that hybridizes to a polymorphic sequence selected shown in Table 1, or its complement, provided that the polymorphic sequence includes a nucleotide other than the nucleotide recited in Table 1, column 5 for the polymorphic sequence, or the complement includes a nucleotide other than the complement of the nucleotide recited in Table 1, column 5. The method also includes determining whether the nucleic acid and the oligonucleotide hybridize. Hybridization of the oligonucleotide to the nucleic acid sequence indicates the presence of the polymorphic site in the nucleic acid.

In preferred embodiments, the oligonucleotide does not hybridize to the polymorphic sequence when the polymorphic sequence includes the nucleotide recited in Table 1, column 5 for the polymorphic sequence, or when the complement of the polymorphic sequence includes the complement of the nucleotide recited in Table 1, column 5 for the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

In some embodiments, the polymorphic sequence identified by the oligonucleotide is associated with a nucleic acid encoding polypeptide related to one of the protein families disclosed herein, the polymorphic sequence is associated with a polypeptide related to one of the protein families disclosed herein. For example, the nucleic acid may be associated with a polypeptide related to angiopoietin, 4-hydroxybutyrate dehydrogenase, or any of the other proteins identified in Table 1, column 10.

In a further aspect, the invention provides a method of determining the relatedness of a first and second nucleic acid. The method includes providing a first nucleic acid and a second nucleic acid and contacting the first nucleic acid and the second nucleic acid with an oligonucleotide that hybridizes to a polymorphic sequence selected disclosed in Table 1, or its complement, provided that the polymorphic sequence includes a nucleotide other than the nucleotide recited in Table 1, column 5 for the polymorphic sequence, or the complement includes a nucleotide other than the complement of the nucleotide recited in Table 1, column 5. The method also includes determining whether the first nucleic acid and the second nucleic acid hybridize to the oligonucleotide, and comparing hybridization of the first and second nucleic acids to the oligonucleotide. Hybridization of first and second nucleic acids to the nucleic acid indicates the first and second subjects are related.

In preferred embodiments, the oligonucleotide does not hybridize to the polymorphic sequence when the polymorphic sequence includes the nucleotide recited in Table 1, column 5 for the polymorphic sequence, or when the complement of the polymorphic sequence includes the complement of the nucleotide recited in Table 1, column 5 column for the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

The method can be used in a variety of applications. For example, the first nucleic acid may be isolated from physical evidence gathered at a crime scene, and the second nucleic acid may be obtained is a person suspected of having committed the crime. Matching the two nucleic acids using the method can establishing whether the physical evidence originated from the person.

In another example, the first sample may be from a human male suspected of being the father of a child and the second sample may be from a child. Establishing a match using the described method can establishing whether the male is the father of the child.

In another aspect, the method includes determining if a sequence polymorphism is the present in a subject, such as a human. The method includes providing a nucleic acid from the subject and contacting the nucleic acid with an oligonucleotide that hybridizes to a polymorphic sequence disclosed in Table 1, or its complement, provided that the polymorphic sequence includes a nucleotide other than the nucleotide recited in Table 1, column 5 for the polymorphic sequence, or the complement includes a nucleotide other than the complement of the nucleotide recited in Table 1, column 5. Hybridization between the nucleic acid and the oligonucleotide is then determined. Hybridization of the oligonucleotide to the nucleic acid sequence indicates the presence of the polymorphism in said subject.

In another aspect, the invention provides an isolated polypeptide comprising a polymorphic site at one or more amino acid residues, and wherein the protein is encoded by a polynucleotide including one of the polymorphic sequences in Table 1, or their complement, provided that the polymorphic sequence includes a nucleotide other than the nucleotide recited in Table 1, column 5 for the polymorphic sequence, or the complement includes a nucleotide other than the complement of the nucleotide recited in Table 1, column 5.

The polypeptide can be, e.g., related to one of the protein families disclosed herein. For example, polypeptide can be related to angiopoietin, 4-hydroxybutyrate dehydrogenase, ATP-dependent RNA helicase, MHC Class I histocompatibility antigen, or phosphoglycerate kinase.

In some embodiments, the polypeptide is translated in the same open reading frame as is a wild type protein whose amino acid sequence is identical to the amino acid sequence of the polymorphic protein except at the site of the polymorphism.

In some embodiments, the polypeptide encoded by the polymorphic sequence, or its complement, includes the nucleotide listed in Table 1, column 6 for the polymorphic sequence, or the complement includes the complement of the nucleotide listed in Table 1, column 6.

The invention also provides an antibody that binds specifically to a polypeptide encoded by a polynucleotide comprising a nucleotide sequence encoded by a polynucleotide including one or more of the polymorphic sequences in Table 1, or its complement. The polymorphic sequence includes a nucleotide other than the nucleotide recited in Table 1, column 5 for the polymorphic sequence, or the complement includes a nucleotide other than the complement of the nucleotide recited in Table 1, column 5.

In some embodiments, the antibody binds specifically to a polypeptide encoded by a polymorphic sequence which includes the nucleotide listed in Table 1, column 6 for the polymorphic sequence.

Preferably, the antibody does not bind specifically to a polypeptide encoded by a polymorphic sequence which includes the nucleotide listed in Table 1, column 5 for the polymorphic sequence.

The invention further provides a method of detecting the presence of a polypeptide having one or more amino acid residue polymorphisms in a subject. The method includes providing a protein sample from the subject and contacting the sample with the above-described antibody under conditions that allow for the formation of antibody-antigen complexes. The antibody-antigen complexes are then detected. The presence of the complexes indicates the presence of the polypeptide.

The invention also provides a method of treating a subject suffering from, at risk for, or suspected of, suffering from a pathology ascribed to the presence of a sequence polymorphism in a subject, e.g., a human, non-human primate, cat, dog, rat, mouse, cow, pig, goat, or rabbit. The method includes providing a subject suffering from a pathology associated with aberrant expression of a first nucleic acid comprising a polymorphic sequence shown in Table 1, or its complement, and treating the subject by administering to the subject an effective dose of a therapeutic agent. Aberrant expression can include qualitative alterations in expression of a gene, e.g., expression of a gene encoding a polypeptide having an altered amino acid sequence with respect to its wild-type counterpart. Qualitatively different polypeptides can include, shorter, longer, or altered polypeptides relative to the amino acid sequence of the wild-type polypeptide. Aberrant expression can also include quantitative alterations in expression of a gene. Examples of quantitative alterations in gene expression include lower or higher levels of expression of the gene relative to its wild-type counterpart, or alterations in the temporal or tissue-specific expression pattern of a gene. Finally, aberrant expression may also include a combination of qualitative and quantitative alterations in gene expression.

The therapeutic agent can include, e.g., second nucleic acid comprising the polymorphic sequence, provided that the second nucleic acid comprises the nucleotide present in the wild type allele. In some embodiments, the second nucleic acid sequence comprises a polymorphic sequence which includes nucleotide listed in Table 1, column 5 for the polymorphic sequence.

Alternatively, the therapeutic agent can be a polypeptide encoded by a polynucleotide comprising polymorphic sequence shown in Table 1, or by a polynucleotide comprising a nucleotide sequence that is complementary to any one of the polymorphic sequences, provided that the polymorphic sequence includes the nucleotide listed in Table 1, column 6 for the polymorphic sequence.

The therapeutic agent may further include an antibody as herein described, or an oligonucleotide comprising a polymorphic sequence shown in Table 1, or by a polynucleotide comprising a nucleotide sequence that is complementary to any one the polymorphic sequences, provided that the polymorphic sequence includes the nucleotide listed in Table 1, column 6 for the polymorphic sequence,

In another aspect, the invention provides an oligonucleotide array comprising one or more oligonucleotides hybridizing to a first polynucleotide at a polymorphic site encompassed therein. The first polynucleotide can be, e.g., a nucleotide sequence comprising one or more polymorphic sequences shown in Table 1; a nucleotide sequence that is a fragment of any of the nucleotide sequence, provided that the fragment includes a polymorphic site in the polymorphic sequence; a complementary nucleotide sequence comprising a sequence complementary to one or more of the polymorphic sequences; or a nucleotide sequence that is a fragment of the complementary sequence, provided that the fragment includes a polymorphic site in the polymorphic sequence.

In preferred embodiments, the array comprises 10; 100; 1,000; 10,000; 100,000 or more oligonucleotides.

The invention also provides a kit comprising one or more of the herein-described nucleic acids. The kit can include, e.g., polynucleotide which includes one or more of the SNPs described herein. The polynucleotide can be, e.g., a nucleotide sequence which includes one or more of the polymorphic sequences shown in Table 1, and which includes a polymorphic sequence, or a fragment of the polymorphic sequence, as long as it includes the polymorphic site. The polynucleotide may alternatively contain a nucleotide sequence which includes a sequence complementary to one or more of the sequences, or a fragment of the complementary nucleotide sequence, provided that the fragment includes a polymorphic site in the polymorphic sequence.

Alternatively, or in addition, the kit can include the invention provides an isolated allele-specific oligonucleotide that hybridizes to a first polynucleotide containing a polymorphic site. The first polynucleotide can be, e.g., a nucleotide sequence comprising one or more polymorphic sequences shown in Table 1, provided that the polymorphic sequence includes a nucleotide other than the nucleotide recited in Table 1, column 5 for the polymorphic sequence. Alternatively, the first polynucleotide can be a nucleotide sequence that is a fragment of the polymorphic sequence, provided that the fragment includes a polymorphic site in the polymorphic sequence, or a complementary nucleotide sequence which includes a sequence complementary to one or more polymorphic sequences shown in Table 1, provided that the complementary nucleotide sequence includes a nucleotide other than the complement of the nucleotide recited in Table 1, column 6. The first polynucleotide may in addition include a nucleotide sequence that is a fragment of the complementary sequence, provided that the fragment includes a polymorphic site in the polymorphic sequence.

### BRIEF DESCRIPTION OF THE DRAWING

FIG. 1 illustrates an example of the way in which SNP sites were identified in the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides human SNPs in sequences which are transcribed, i.e., are cSNPs. Many SNPs have been identified in genes related to polypeptides of known function. If desired, SNPs associated with various polypeptides can be used together. For example, SNPs can be grouped according to whether they are derived from a nucleic acid encoding a polypeptide related to particular protein family or involved in a particular function. Similarly, SNPs can be grouped according to the functions played by their gene products. Such functions include, structural proteins, proteins from which associated with metabolic pathways fatty acid metabolism, glycolysis, intermediary metabolism, calcium metabolism, proteases, and amino acid metabolism, etc. Specifically, the present invention provides a large number of human cSNP's based on at least one gene product that has not been previously identified. In contrast, and as defined specifically in the following paragraph, the cSNP's involve nucleic acid sequences that are assembled from at least one known sequence.

The present invention describes 651 distinct polymorphic sites, which are summarized in Table 1. Raw traces underlying sequence data were drawn from public databases and from the proprietary database of the Assignee of the present invention. The sequences were obtained by calling the bases from these traces, and included assigning “Phred” quality scores for each called base. For each allelic set, at the polynucleotide level, four or more nucleotide sequences were identified having at least partial overlap with one another.

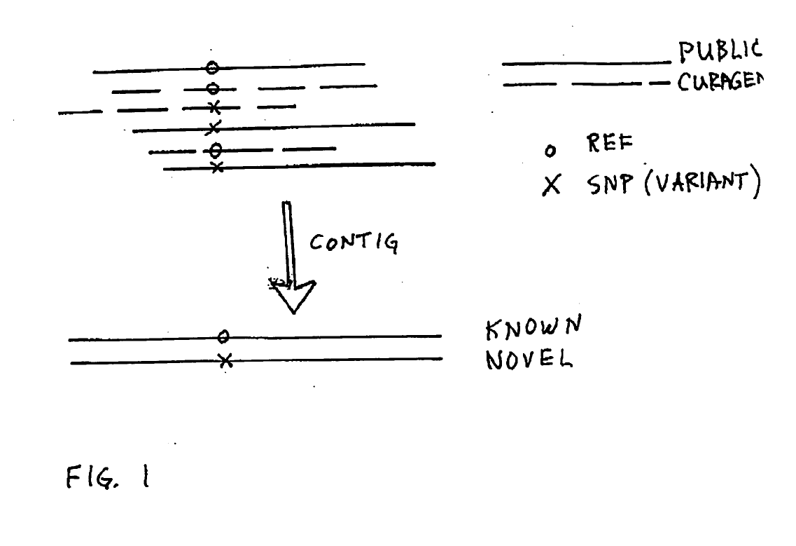

As illustrated in FIG. 1, these four or more sequences could be clustered and assembled to make a consensus contig that included an ORF. In this way, the inventors found that the assembled contigs defined associated sets of two, or possibly more than two, alleles defined by a SNP at a particular polymorphic site. In order to be confirmed as a SNP site, the nucleotide change from the consensus sequence had to occur in at least two individual sequences, and had to have a “Phred” score of 23 or higher at the site of the presumed SNP. Furthermore, in a window of 5 bases on either side of the SNP, no more than 50% mismatching with the consensus sequence was allowed. In the assembly leading to each of the contigs defining the allelic set, the SNP alleles occur in polynucleotides found in public databases.

It was found that the assembled contigs defined associated sets of two, or possibly more than two, alleles defined by an SNP at a particular polymorphic site. These associations were not previously known.

At the level of translation of an ORF contained in the contigs, allelic sets were identified in which one allele defines a known polypeptide sequence that includes the polymorphic site and another polypeptide allele is not previously known. Then, various associations of alleles are possible. For example, it is possible that an allelic pair is defined in a noncoding region of the contig containing an ORF. In such cases the inventors believe that the invention resides in the recognition of the allelic pair; this association has not heretofore been made.

Alternatively, sets of allelic contigs may exist in which the polymorphic site is within an ORF, but does not result in an amino acid change among the allelic polypeptides. Thus, in another embodiment, the polymorphic site resides within an ORF and results in an amino acid change, or a frameshift, among the alleles of the allelic set. In the sets of gene products that fall within this group, at least one of the alleles at the polypeptide level is a known protein. At least one of the remaining allele or alleles in the set, carrying a variant amino acid at the polymorphic site, is a novel polypeptide not heretofore known. The invention resides at least in the recognition of the polymorphic allele as being a variant of the known reference polypeptide.

Table 1 provides information concerning the allelic sequences. One of the sequences may be termed a reference polymorphic sequence, and the corresponding second sequence includes the variant SNP at the polymorphic site. Since the reference polypeptide sequence is already known, the Sequence Listing accompanying this application provides only the sequence of the polymorphic allele, while its SEQ ID NO is provided in the Table. A reference to the SEQ ID NO that corresponds to the translated amino acid sequence is also given. The Table includes thirteen columns that provide descriptive information for each cSNP, each of which occupies one row in the Table. The column headings, and a description of each, are given below.

SNPs disclosed in Table 1 were detected by aligning large numbers of sequences from genetically diverse sources of publicly available mRNA libraries (Clontech). Software designed specifically to look for multiple examples of variant bases differing from a consensus sequence was created and deployed. A criteria of a minimum of 2 occurrences of a sequence differing from the consensus in high quality sequence reads was used to identify an SNP.

The SNPs described herein may be useful in diagnostic kits, for DNA arrays on chips and for other uses that involve hybridization of the SNP.

Specific SNPs may have utility where a disease has already been associated with that gene. Examples of possible disease correlations between the claimed SNPs with members of the genes of each classification are listed below:

Amylases

Amylase is responsible for endohydrolysis of 1,4-alpha-glucosidic linkages in oligosaccharides and polysaccharides. Variations in amylase gene may be indicative of delayed maturation and of various amylase producing neoplasms and carcinomas.

Amyloid

The serum amyloid A (SAA) proteins comprise a family of vertebrate proteins that associate predominantly with high density lipoproteins (HDL). The synthesis of certain members of the family is greatly increased in inflammation. Prolonged elevation of plasma SAA levels, as in chronic inflammation, 15 results in a pathological condition, called amyloidosis, which affects the liver, kidney and spleen and which is characterized by the highly insoluble accumulation of SAA in these tissues. Amyloid selectively inhibits insulin-stimulated glucose utilization and glycogen deposition in muscle, while not affecting adipocyte glucose metabolism. Deposition of fibrillar amyloid proteins intraneuronally, as neurofibrillary tangles, extracellularly, as plaques and in blood vessels, is characteristic of both Alzheimer's disease and aged Down's syndrome. Amyloid deposition is also associated with type II diabetes mellitus.

Angiopoeitin

Members of the angiopoeitin/fibrinogen family have been shown to stimulate the generation of new blood vessels, inhibit the generation of new blood vessels, and perform several roles in blood clotting. This generation of new blood vessels, called angiogenesis, is also an essential step in tumor growth in order for the tumor to get the blood supply it needs to expand. Variation in these genes may be predictive of any form of heart disease, numerous blood clotting disorders, stroke, hypertension and predisposition to tumor formation and metastasis. In particular, these variants may be predictive of the response to various antihypertensive drugs and chemotherapeutic and anti-tumor agents.

Apoptosis-Related Proteins

Active cell suicide (apoptosis) is induced by events such as growth factor withdrawal and toxins. It is controlled by regulators, which have either an inhibitory effect on programmed cell death (anti-apoptotic) or block the protective effect of inhibitors (pro-apoptotic). Many viruses have found a way of countering defensive apoptosis by encoding their own anti-apoptosis genes preventing their target-cells from dying too soon. Variants of apoptosis related genes may be useful in formulation of anti-aging drugs.

Cadherin, Cyclin, Polymerase, Oncogenes, Histones, Kinases

Members of the cell division/cell cycle pathways such as cyclins, many transcription factors and kinases, DNA polymerases, histones, helicases and other oncogenes play a critical role in carcinogenesis where the uncontrolled proliferation of cells leads to tumor formation and eventually metastasis. Variation in these genes may be predictive of predisposition to any form of cancer, from increased risk of tumor formation to increased rate of metastasis. In particular, these variants may be predictive of the response to various chemotherapeutic and anti-tumor agents.

Colony-Stimulating Factor-Related Proteins

Granulocyte/macrophage colony-stimulating factors are cytokines that act in hematopoiesis by controlling the production, differentiation, and function of 2 related white cell populations of the blood, the granulocytes and the monocytes-macrophages.

Complement-Related Proteins

Complement proteins are immune associated cytotoxic agents, acting in a chain reaction to exterminate target cells to that were opsonized (primed) with antibodies, by forming a membrane attack complex (MAC). The mechanism of killing is by opening pores in the target cell membrane. Variations in 20 complement genes or their inhibitors are associated with many autoimmune disorders. Modified serum levels of complement products cause edemas of various tissues, lupus (SLE), vasculitis, glomerulonephritis, renal failure, hemolytic anemia, thrombocytopenia, and arthritis. They interfere with mechanisms of ADCC (antibody dependent cell cytotoxicity), severely impair immune competence and reduce phagocytic ability. Variants of complement genes may also be indicative of type I diabetes mellitus, meningitis neurological disorders such as Nemaline myopathy, Neonatal hypotonia, muscular disorders such as congenital myopathy and other diseases.

Cytochrome

The respiratory chain is a key biochemical pathway which is essential to all aerobic cells. There are five different cytochromes involved in the chain. These are heme bound proteins which serve as electron carriers. Modifications in these genes may be predictive of ataxia areflexia, dementia and myopathic and neuropathic changes in muscles. Also, association with various types of solid tumors.

Kinesins

Kinesins are tubulin molecular motors that function to transport organelles within cells and to move chromosomes along microtubules during cell division. Modifications of these genes may be indicative of neurological disorders such as Pick disease of the brain, tuberous sclerosis.

Cytokines, Interferon, Interleukin

Members of the cytokine families are known for their potent ability to stimulate cell growth and division even at low concentrations. Cytokines such as erythropoietin are cell-specific in their growth stimulation; erythropoietin is useful for the stimulation of the proliferation of erythroblasts. Variants in cytokines may be predictive for a wide variety of diseases, including cancer predisposition.

G-Protein Coupled Receptors

G-protein coupled receptors (also called R7G) are an extensive group of hormones, neurotransmitters, odorants and light receptors which transduce extracellular signals by interaction with guanine nucleotide-binding (G) proteins. Alterations in genes coding for G-coupled proteins may be involved in and indicative of a vast number of physiological conditions. These include blood pressure regulation, renal dysfunctions, male infertility, dopamine associated cognitive, emotional, and endocrine functions, hypercalcemia, chondrodysplasia and osteoporosis, pseudohypoparathyroidism, growth retardation and dwarfism.

Thioesterases

Eukaryotic thiol proteases are a family of proteolytic enzymes which contain an active site cysteine. Catalysis proceeds through a thioester intermediate and is facilitated by a nearby histidine side chain; an asparagine completes the essential catalytic triad. Variants of thioester associated genes may be predictive of neuronal disorders and mental illnesses such as Ceroid Lipoffiscinosis, Neuronal 1, Infantile, Santavuori disease and more.

Breakdown Classifications of SNPS

The following list describes the numerical breakdown by molecule type of the SNPs described in Table 1. The key to these molecule types is as follows.

| | |
|---|---|
| TPase_associated: | 864 |
| Guanylyl: | 3 |
| MHC: | 1077 |
| amylase: | 44 |
| amylaseinhib: | 1 |
| amyloid: | 96 |
| apoptosis: | 91 |
| apoptosisinhib: | 29 |
| apoptosisrecep: | 14 |
| biotindep: | 29 |
| cadhenn: | 415 |
| calcium_channel: | 85 |
| carboxylase: | 4 |
| cathepsin: | 336 |
| cathepsininhib: | 41 |
| chloride_channel: | 90 |
| collagen: | 1542 |
| complement: | 222 |
| complementinhib: | 21 |
| complementrecept: | 10 |
| csf: | 31 |
| csf recept: | 37 |
| cyclin: | 65 |
| cyto450: | 136 |
| cytochrome: | 659 |
| deaminase: | 44 |
| dehydrogenase: | 1235 |
| desaturase: | 9 |
| dna_rna_bind: | 1309 |
| dna_rna_bind_inhib: | 16 |
| dynein: | 108 |
| elastase: | 134 |
| elastaseinhib: | 6 |
| eph: | 487 |
| esterase: | 258 |
| esteraseinhib: | 3 |
| fgf: | 34 |
| fgf receptor: | 12 |
| gaba: | 45 |
| glucoamylase: | 106 |
| glucuronidase: | 14 |
| glycoprotein: | 3176 |
| helicase: | 333 |
| histone: | 272 |
| homeobox: | 431 |
| hydrolase: | 187 |
| hydroxysteroid: | 84 |
| hypoxanthine: | 4 |
| immunoglob: | 1106 |
| immunoglob_recept: | 19 |
| interferon: | 322 |
| interleukin: | 88 |
| interleukinrecept: | 126 |
| isomerase: | 404 |
| isomeraseinhibitor: | 45 |
| isomerasereceptor: | 4 |
| kinase: | 1684 |
| kinase inhibitor: | 187 |
| kinase receptor: | 233 |
| kinesin: | 86 |
| laminin: | 196 |
| lipase: | 63 |
| metallothionein: | 62 |
| misc_channel: | 215 |
| ngf: | 30 |
| nucl_recpt: | 339 |
| nuclease: | 298 |
| oncogene: | 783 |
| oxidase: | 128 |
| oxygenase: | 14 |
| peptidase: | 150 |
| peroxidase: | 115 |
| phosphatase: | 668 |
| phosphataseinhib: | 71 |
| phosphorylase: | 84 |
| polymerase: | 489 |
| potassium_channel: | 43 |
| prostaglandin: | 55 |
| protease: | 954 |
| proteaseinhib: | 271 |
| reductase: | 243 |
| ribosomal prot: | 1040 |
| struct: | 3128 |
| sulfotransferase: | 42 |
| synthase: | 893 |
| tgf: | 117 |
| tgfreceptor: | 41 |
| thioesterase: | 3 |
| thiolase: | 38 |
| tm7: | 453 |
| tnf: | 151 |
| tnfreceptor: | 36 |
| traffic: | 22 |
| transcriptfactor: | 1139 |
| transferase: | 291 |
| transport: | 900 |
| tubulin: | 334 |
| ubiquitin: | 229 |
| water_channel: | 18 |
| unclassified: | 10567 |

TABLE 1
The key to the molecule type is as follows:

| Abbrev: | Title: |
|---|---|
| amylase | amylase protein |
| amylaseinhib | amylase inhibitor |
| amyloid | amyloid protein |
| apoptosis | apoptosis associated protein |
| apoptosisinhib | apoptosis inhibitors |
| apoptosisrecep | apoptosis receptors |
| ATPase_associated | ATPase associated protein |
| biotindep | biotin dependent enzyme/protein |
| cadherin | cadherin protein |
| calcium_channel | calcium channel protein |
| carboxylase | carboxylase protein |
| cathepsin | cathepsin/carboxypeptidases |
| cathepsininhib | cathepsin/carboxypeptidase inhibitor |
| chloride_channel | chloride channel protein |
| collagen | collagen |
| complement | complement protein |
| complementrecept | complement receptor protein |
| complementinhib | complement inhibitor |
| csf | colony stimulating factor |
| csfrecept | colony stimulating factor receptor |
| cyclin | cyclin protein |
| cyto450 | cytochrome p450 protein |
| cytochrome | cytochrome related protein |
| deaminase | deaminase |
| dehydrogenase | dehydrogenase |
| desaturase | desaturase |
| dna_rna_bind | DNA/RNA binding protein/factor |
| dna_rna_inhib | DNA/RNA binding protein/factor |
| | inhibitor |
| dynein | dynein |
| elastase | elastase |
| elastaseinhib | elastase inhibitor |
| eph | EPH family of tyrosine kinases |
| esterase | esterase |
| esteraseinhib | esterase inhibitor |
| fgf | fibroblast growth factor |
| fgfreceptor | fibroblast growth factor receptor |
| gaba | GABA receptor |
| glucoamylase | glucoamylase |
| glucoronidase | glucoronidase |
| glycoprotein | glycoprotein |
| Guanylyl | guanylylate cyclase |
| helicase | helicase |
| histone | histone |
| HOM | homologous |
| homeobox | homeobox protein |
| hydrolase | hydrolase |
| hydroxysteroid | hydroxysteroid associated protein |
| hypoxanthine | hypoxanthine associated protein |
| immunoglob | immunoglobulin |
| immunoglobrecept | immunoglobulin receptor |
| interferon | interferon |
| interleukin | interleukin |
| interleukinrecept | interleukin receptor |
| isomerase | isomerase |
| isomeraseinhibitor | isomerase inhibitor |
| isomerasereceptor | isomerase receptor |
| kinase | kinase |
| kinaseinhibitor | kinase inhibitor |
| kinasereceptor | kinase receptor |
| kinesin | kinesin |
| laminin | laminin associated protein |
| lipase | lipase |
| metallothionein | metallothionein |
| MHC | major histocompatability complex |
| misc_channel | miscellaneous channel |
| ngf | nerve growth factor |
| nuci_recpt | nuclear receptor |
| nuclease | nuclease |
| oncogene | oncogene associated protein |
| oxidase | oxidase |
| oxygenase | oxygenase |
| peptidase | peptidase |
| peroxidase | peroxidase |
| phosphatase | phosphatase |
| phosphataseinhib | phosphatase inhibitor |
| phosphorylase | phosphorylase |
| PIR | PIR DATABASE (release 56, 29- |
| | OCT-1998) |
| polymerase | polymerase |
| potassium_channel | potassium channel protein |
| prostaglandin | prostaglandin |
| protease | protease |
| proteaseinhib | protease inhibitor |
| reductase | reductase |
| ribosomalprot | ribosomal associated protein |
| RTR | EMBLDATABASE translated |
| | entries not to be incorporated into |
| | SWISS-PROT (20-JUL-1998) |
| SIM | similar |
| SPTR | EMBL DATABASE translated |
| | entries to be incorporated into |
| | SWISS-PROT (20-JUL-1998) |
| struct | structural associated protein |
| sulfotransferase | sulfotransferase |
| SWP | SWISS-PROT DATABASE (release |
| | 18-OCT-1998) |
| SWPN | SWISS-PROT Update (release 11- |
| | NOV-98) |
| synthase | synthase |
| tgf | transforming growth factor |
| tgfreceptor | transforming growth factor receptor |
| thioesterase | thioesterase |
| thiolase | thiolase |
| tm7 | seven transmembrane domain G- |
| | protein coupled receptor |
| tnf | necrosis factor receptor |
| traffic | tumor necrosis factor |
| tnfreceptor | tumor trafficking associated protein |
| TRN | EMBL DATABASE translated |
| | entries update (20-JUL-1998) |
| transcriptfactor | transcription factor |
| transferase | transferase |
| transport | transport protein |
| tubulin | tubulin |
| ubiquitin | ubiquitin |
| unclassified | Protein not categorized into one of |
| | the aforementioned protein families |
| water channel | water channel protein |

A compilation of polymorphisms is listed in Table 1. Table 1 includes thirteen columns that provide descriptive information for each cSNP, each of which occupies one row in the Table. The column headings, and an explanation for each, are given below.

The first column of the table lists the names assigned to the fragments in which the polymorphisms occur. The fragments are all human genomic fragments. The sequence of one allelic form of each of the fragments (arbitrarily referred to as the prototypical or reference form ) has been previously published. These sequences are listed at http://www-genome.wi.mit.edu/ (all STS's sequence tag sites)); http://shgc.stanford.edu (Stanford STS's); and http://www.tigr.org/ (TIGR STS's). The web sites also list primers for amplification of the fragments, and the genomic location of the fragments. Some fragments arc expressed sequence tags, and some are random genomic fragments. All information in the web sites concerning the fragments listed in the table is incorporated by reference in its entirety for all purposes.

The second column lists the position in the fragment in which a polymorphic site has been found. Positions are numbered consecutively with the first base of the fragment sequence listed as in one of the above databases being assigned the number one. The third column lists the base occupying the polymorphic site in the sequence in the data base. This base is arbitrarily designated the reference or prototypical form, but it is not necessarily the most frequently occurring form. The fourth column in the table lists the alternative base(s) at the polymorphic site. The fifth column of the table lists a 5′ (upstream or forward) primer that hybridizes with the 5′ end of the DNA sequence to be amplified. The sixth column of the table lists a 3′ (downstream or reverse) primer that hybridizes with the complement of the 3′ end of the sequence to be amplified. The seventh column of the table lists a number of bases of sequence on either side of the polymorphic site in each fragment. The indicated sequences can either be DNA or RNA. In the latter, the T's shown in the table are replaced by U's. The base occupying the polymorphic site is indicated in EUT′AC-IUB ambiguity code.

“SEQ ID” provides the cross-references to the two nucleotide SEQ ID NOS: for the cognate pair, which are numbered consecutively, and, as explained below, amino acid SEQ ID NOS: as well, in the Sequence Listing of the application.

Each sequence entry in the Sequence Listing also includes a cross-reference to the CuraGen sequence ID, under the label “Accession number”. The first pair of SEQ ID NOS: given in the first column of each row of the Table is the SEQ ID NO: identifying the nucleic acid sequence for the polymorphism. If a polymorphism carries an entry for the amino acid portion of the row, a third SEQ ID NO: appears in parentheses in the column “Amino acid before” (see below) for the reference amino acid sequence, and a fourth SEQ ID NO: appears in parentheses in the column “Amino acid after” (see below) for the polymorphic amino acid sequence. The latter SEQ ID NOS: refer to amino acid sequences giving the cognate reference and polymorphic amino acid sequences that are the translation of the nucleotide polymorphism. If a polymorphism carries no entry for the protein portion of the row, only one pair SEQ ID NOS: is provided, in the first column.

“CuraGen sequence ID” provides CuraGen Corporation's accession number.

“Base pos. of SNP” gives the numerical position of the nucleotide in the nucleic acid at which the cSNP is found, as identified in this invention.

“Polymorphic sequence” provides a 51-base sequence with the polymorphic site at the 26^{th} base in the sequence, as well as 25 bases from the reference sequence on the 5′ side and the 3′ side of the polymorphic site. The designation at the polymorphic site is enclosed in square brackets, and provides first, the reference nucleotide; second, a “slash (/)”; and third, the polymorphic nucleotide. In certain cases the polymorphism is an insertion or a deletion. In that case, the position that is “unfilled” (i.e., the reference or the polymorphic position) is indicated by the word “gap”.

“Base before” provides the nucleotide present in the reference sequence at the position at which the polymorphism is found.

“Base after” provides the altered nucleotide at the position of the polymorphism.

“Amino acid before” provides the amino acid in the reference protein, if the polymorphism occurs in a coding region. This column also includes the SEQ ID NO: in parentheses for the translated reference amino acid sequence if the polymorphism occurs in a coding region.

“Amino acid after” provides the amino acid in the polymorphic protein, if the polymorphism occurs in a coding region. This column also includes the SEQ ID NO in parentheses for the translated polymorphic amino acid sequence if the polymorphism occurs in a coding region.

“Type of change” provides information on the nature of the polymorphism. “SILENT-NONCODING” is used if the polymorphism occurs in a noncoding region of a nucleic acid. “SILENT-CODING” is used if the polymorphism occurs in a coding region of a nucleic acid of a nucleic acid and results in no change of amino acid in the translated polymorphic protein. “CONSERVATIVE” is used if the polymorphism occurs in a coding region of a nucleic acid and provides a change in which the altered amino acid falls in the same class as the reference amino acid. The classes are: 1) Aliphatic: Gly, Ala, Val, Leu, Ile; 2) Aromatic: Phe, Tyr, Trp; 3) Sulfur-containing: Cys, Met; 4) Aliphatic OH: Ser, Thr; 5) Basic: Lys, Arg, His; 6) Acidic: Asp, Glu, Asn, Gln; 7) Pro falls in none of the other classes; and 8) End defines a termination codon.

“NONCONSERVATIVE” is used if the polymorphism occurs in a coding region of a nucleic acid and provides a change in which the altered amino acid falls in a different class than the reference amino acid.

“FRAMESHIFT” relates to an insertion or a deletion. If the frameshift occurs in a coding region, the Table provides the translation of the frameshifted codons 3′ to the polymorphic site.

“Protein classification of CuraGen gene” provides a generic class into which the protein is classified. Multiple classes of proteins were identified as listed above in the discussion of Table 1.

“Name of protein identified following a BLASTX analysis of the CuraGen sequence” provides the database reference for the protein found to resemble the novel reference-polymorphism cognate pair most closely.

“Similarity (pvalue) following a BLASTX analysis” provides the pvalue, a statistical measure from the BLASTX analysis that the polymorphic sequence is similar to, and therefore an allele of, the reference, or wild-type, sequence. In the present application, a cutoff of pvalue>1×10⁻⁵⁰ (entered, for example, as 1.0E-50 in the Table) is used to establish that the reference-polymorphic cognate pairs are novel. A pvalue<1×10⁻⁵⁰ defines proteins considered to be already known.

“Map location” provides any information available at the time of filing related to localization of a gene on a chromosome.

The polymorphisms are arranged in Table 1 in the following order:

SEQ ID NOs: 1-422 are nucleotide sequences for SNPs that are silent.

SEQ ID NOs: 423-480 are nucleotide sequences for SNPs that lead to conservative amino acid changes.

SEQ ID NOs: 481-619 are nucleotide sequences for SNPs that lead to nonconservative amino acid changes.

SEQ ID NOs: 620-651 are nucleotide sequences for SNPs that involve a gap. With respect to the reference or wild-type sequence at the position of the polymorphism, the allelic cSNP introduces an additional nucleotide (an insertion) or deletes a nucleotide (a deletion). An SNP that involves a gap generates a frame shift.

Also presented in the sequence listing filed herewith are predicted amino acid sequences encoded by the polymorphic sequences shown in Table 1.

SEQ ID NOs: 652-709 are the amino acid sequences centered at the polymorphic amino acid residue for the protein products provided by SNPs that lead to conservative amino acid changes. 7 or 8 amino acids on either side of the polymorphic site are shown. The order in which these sequences appear mirrors the order of presentation of the cognate nucleotide sequences, and is set forth in the Table.

SEQ ID NOs: 710-848 are the amino acid sequences centered at the polymorphic amino acid residue for the protein products provided by SNPs that lead to nonconservative amino acid changes. 7 or 8 amino acids on either side of the polymorphic site are shown. The order in which these sequences appear mirrors the order of presentation of the cognate nucleotide sequences, and is set forth in the Table.

SEQ ID NOs: 849-880 are the amino acid sequences centered at the polymorphic amino acid residue for the protein products provided by SNPs that lead to frameshift-induced amino acid changes. 7 or 8 amino acids on either side of the polymorphic site are shown. The order in which these sequences appear mirrors the order of presentation of the cognate nucleotide sequences, and is set forth in the Table.

Provided herein are compositions which include, or are capable of detecting, nucleic acid sequences having these polymorphisms, as well as methods of using nucleic acids.

Identification of Individuals Carrying SNPs

Individuals carrying polymorphic alleles of the invention may be detected at either the DNA, the RNA, or the protein level using a variety of techniques that are well known in the art. Strategies for identification and detection are described in e.g., EP 730,663, EP 717,113, and PCT US97/02102. The present methods usually employ pre-characterized polymorphisms. That is, the genotyping location and nature of polymorphic forms present at a site have already been determined. The availability of this information allows sets of probes to be designed for specific identification of the known polymorphic forms.

Many of the methods described below require amplification of DNA from target samples. This can be accomplished by e.g., PCR. (1989), B. for detecting polymorphisms. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

The phrase “recombinant protein” or “recombinantly produced protein” refers to a peptide or protein produced using non-native cells that do not have an endogenous copy of DNA able to express the protein. In particular, as used herein, a recombinantly produced protein relates to the gene product of a polymorphic allele, i.e., a “polymorphic protein” containing an altered amino acid at the site of translation of the nucleotide polymorphism. The cells produce the protein because they have been genetically altered by the introduction of the appropriate nucleic acid sequence. The recombinant protein will not be found in association with proteins and other subcellular components normally associated with the cells producing the protein. The terms “protein” and “polypeptide” are used interchangeably herein.

The phrase “substantially purified” or “isolated” when referring to a nucleic acid, peptide or protein, means that the chemical composition is in a milieu containing fewer, or preferably, essentially none, of other cellular components with which it is naturally associated. Thus, the phrase “isolated” or “substantially pure” refers to nucleic acid preparations that lack at least one protein or nucleic acid normally associated with the nucleic acid in a host cell. It is preferably in a homogeneous state although it can be in either a dry or aqueous solution. Purity and homogeneity are typically determined using analytical chemistry techniques such as gel electrophoresis or high performance liquid chromatography. Generally, a substantially purified or isolated nucleic acid or protein will comprise more than 80% of all macromolecular species present in the preparation. Preferably, the nucleic acid or protein is purified to represent greater than 90% of all macromolecular species present. More preferably the nucleic acid or protein is purified to greater than 95%, and most preferably the nucleic acid or protein is purified to essential homogeneity, wherein other macromolecular species are not detected by conventional analytical procedures.

The genomic DNA used for the diagnosis may be obtained from any nucleated cells of the body, such as those present in peripheral blood, urine, saliva, buccal samples, surgical specimen, and autopsy specimens. The DNA may be used directly or may be amplified enzymatically in vitro through use of PCR (Saiki et al. Science 239:487-491 (1988)) or other in vitro amplification methods such as the ligase chain reaction (LCR) (Wu and Wallace Genomics 4:560-569 (1989)), strand displacement amplification (SDA) (Walker et al. Proc. Natl. Acad. Sci. U.S.A. 89:392-396 (1992)), self-sustained sequence replication (3SR) (Fahy et al. PCR Methods P&J& 1:25-33 (1992)), prior to mutation analysis.

The method for preparing nucleic acids in a form that is suitable for mutation detection is well known in the art. A “nucleic acid” is a deoxyribonucleotide or ribonucleotide polymer in either single-or double-stranded form, including known analogs of natural nucleotides unless otherwise indicated. The term “nucleic acids”, as used herein, refers to either DNA or RNA. “Nucleic acid sequence” or “polynucleotide sequence” refers to a single-stranded sequence of deoxyribonucleotide or ribonucleotide bases read from the 5′ end to the 3′ end. The direction of 5′ to 3′ addition of nascent RNA transcripts is referred to as the transcription direction; sequence regions on the DNA strand having the same sequence as the RNA and which are beyond the 5′ end of the RNA transcript in the 5′ direction are referred to as “upstream sequences”; sequence regions on the DNA strand having the same sequence as the RNA and which are beyond the 3′ end of the RNA transcript in the 3′ direction are referred to as “downstream sequences”. The term includes both self-replicating plasmids, infectious polymers of DNA or RNA and nonfunctional DNA or RNA. The complement of any nucleic acid sequence of the invention is understood to be included in the definition of that sequence. “Nucleic acid probes” may be DNA or RNA fragments.

The detection of polymorphisms in specific DNA sequences, can be accomplished by a variety of methods including, but not limited to, restriction-fragment-length-polymorphism detection based on allele-specific restriction-endonuclease cleavage (Kan and Dozy Lancet ii:910-912 (1978)), hybridization with allele-specific oligonucleotide probes (Wallace et al. Nucl. Acids Res. 6:3543-3557 (1978)), including immobilized oligonucleotides (Saiki et al. Proc. Natl. Acad. SCI. USA 86:6230-6234 (1969)) or oligonucleotide arrays (Maskos and Southern Nucl. Acids Res 21:2269-2270 (1993)), allele-specific PCR (Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Natl. Acad. Sci. U.S.A. 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. Proc. Natl. w Sci. U.S.A, 8Z4397-4401(1988)) or enzymatic (Youil et al. Proc. Natl. Acad. Sci. U.S.A. 92:87-91 (1995)) cleavage of heteroduplex DNA, methods based on allele specific primer extension (Syvanen et al. Genomics 8:684-692 (1990)), genetic bit analysis (GBA) (Nikiforov et al. &&I Acids 22:4167-4175 (1994)), the oligonucleotide-ligation assay (OLA) (Landegren et al. Science_{—}241:1077 (1988)), the allele-specific ligation chain reaction (LCR) (Barrany Proc. Natl. Acad. Sci. U.S.A. 88:189-1 93 (1991)), gap-LCR (Abravaya et al. Nucl Acids Res 23:675-682 (1995)), radioactive and/or fluorescent DNA sequencing using standard procedures well known in the art, and peptide nucleic acid (PNA) assays (Orum et al., Nucl. Acids Res, 21:5332-5356 (1993); Thiede et al., Nucl. Acids Res. 24:983-984 (1996)).

“Specific hybridization” or “selective hybridization” refers to the binding, or duplexing, of a nucleic acid molecule only to a second particular nucleotide sequence to which the nucleic acid is complementary, under suitably stringent conditions when that sequence is present in a complex mixture (e.g., total cellular DNA or RNA). “Stringent conditions” are conditions under which a probe will hybridize to its target subsequence, but to no other sequences. Stringent conditions are sequence-dependent and are different in different circumstances. Longer sequences hybridize specifically at higher temperatures than shorter ones. Generally, stringent conditions are selected such that the temperature is about 5° C. lower than the thermal melting point (Tm) for the specific sequence to which hybridization is intended to occur at a defined ionic strength and pH. The Tm is the temperature (under defined ionic strength, pH, and nucleic acid concentration) at which 50% of the target sequence hybridizes to the complementary probe at equilibrium. Typically, stringent conditions include a salt concentration of at least about 0.01 to about 1.0 M Na ion concentration (or other salts), at pH 7.0 to 8.3. The temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

“Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al., or Current Protocols in Molecular Biology, F. Ausubel et al., ed., Greene Publishing and Wiley-Interscience, New York (1987).

A perfectly matched probe has a sequence perfectly complementary to a particular target sequence. The test probe is typically perfectly complementary to a portion of the target sequence. A “polymorphic” marker or site is the locus at which a sequence difference occurs with respect to a reference sequence. Polymorphic markers include restriction fragment length polymorphisms, variable number of tandem repeats (VNTR's), hypervariable regions, minisatellites, dinucleotide repeats. trinucleotide repeats, tetranucleotide repeats, simple sequence repeats, and insertion elements such as Alu. The reference allelic form may be, for example, the most abundant form in a population, or the first allelic form to be identified, and other allelic forms are designated as alternative, variant or polymorphic alleles. The allelic form occurring most frequently in a selected population is sometimes referred to as the “wild type” form, and herein may also be referred to as the “reference” form. Diploid organisms may be homozygous or heterozygous for allelic forms. A diallelic polymorphism has two distinguishable forms (i.e., base sequences), and a triallelic polymorphism has three such forms.

As use herein an “oligonucleotide” is a single-stranded nucleic acid ranging in length from 2 to about 60 bases. Oligonucleotides are often synthetic but can also be produced from naturally occurring polynucleotides. A probe is an oligonucleotide capable of binding to a target nucleic acid of complementary sequence through one or more types of chemical bonds, usually through complementary base pairing via hydrogen bond formation. Oligonucleotides probes are often between 5 and 60 bases, and, in specific embodiments, may be between 1040, or 15-30 bases long. An oligonucleotide probe may include natural (i.e. A, G, C, or T) or modified bases (7-deazaguanosine, inosine, etc.). In addition, the bases in an oligonucleotide probe may be joined by a linkage other than a phosphodiester bond, such as a phosphoramidite linkage or a phosphorothioate linkage, or they may be peptide nucleic acids in which the constituent bases are joined by peptide bonds rather than by phosphodiester bonds, so long as it does not interfere with hybridization. Examples of an oligonucleotide are shown in Table 1. Oligonucleotides can be all of a nucleic acid segment as represented in column 4 of Table 1; a nucleic acid sequence which comprises a nucleic acid segment represented in column 4 of Table 1 and additional nucleic acids (present at either or both ends of a nucleic acid segment of column 4); or a portion (fragment) of a nucleic acid segment represented in column 4 of the table which includes a polymorphic site. Preferred polymorphic sites of the invention include segments of DNA or their complements, which include any one of the polymorphic sites shown in the Table. The segments can be between 5 and 250 bases, and, in specific embodiments are between 5-10, 5-20, 10-20, 10-50, 20-50 or 10-100 bases. The polymorphic site can occur within any position of the segment. The segments can be from any of the allelic forms of the DNA shown in the Table.

As used herein, the term “primer” refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not be perfectly complementary to the exact sequence of the template, but should be sufficiently complementary to hybridize with it. The term “primer site” refers to the sequence of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

DNA fragments can be prepared, for example, by digesting plasmid DNA, or by use of PCR. Oligonucleotides for use as primers or probes are chemically synthesized by methods known in the field of the chemical synthesis of polynucleotides, including by way of non-limiting example the phosphoramidite method described by Beaucage and Carruthers, Tetrahedron Lett 22:1859-1 862 (1981) and the triester method provided by Matteucci, et al., J. Am. Chem. Soc., 103:3185 (1981) both incorporated herein by reference. These syntheses may employ an automated synthesizer, as described in Needham-VanDevanter, D. R., et al., Nucleic Acids Res. 12:61596168 (1984). Purification of oligonucleotides may be carried out by either native acrylamide gel electrophoresis or by anion-exchange HPLC as described in Pearson, J. D. and Regnier. F. E., ,J. Chrom,, 255:137-149 (1983). A double stranded fragment may then be obtained, if desired, by annealing appropriate complementary single strands together under suitable conditions or by synthesizing the complementary strand using a DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The sequence of the synthetic oligonucleotide or of any nucleic acid fragment can be can be obtained using either the dideoxy chain termination method or the Maxam-Gilbert method (see Sambrook et al. Molecular Cloning—a Laboratory Manual (2nd Ed.), Vols. 1-3, Cold Spring Harbor Laboratory, Cold Spring Harbor, N.Y., (1989), which is incorporated herein by reference. This manual is hereinafter referred to as “Sambrook et al.”; Zyskind et al., (1988)). Recombinant DNA Laboratory Manual, (Acad. Press, New York). Oligonucleotides useful in diagnostic assays are typically at least 8 consecutive nucleotides in length, and may range upwards of 18 nucleotides in length to greater than 100 or more consecutive nucleotides.

Another aspect of the invention pertains to isolated antisense nucleic acid molecules that are hybridizable to or complementary to the nucleic acid molecule comprising the SNP-containing nucleotide sequences of the invention, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, about 25, about 50, or about 60 nucleotides or an entire SNP coding strand, or to only a portion thereof.

In one embodiment, an antisense nucleic acid molecule is antisense to a “coding region” of the coding strand of a polymorphic nucleotide sequence of the invention. The term “coding region” refers to the region of the nucleotide sequence comprising codons which are translated into amino acid. In another embodiment, the antisense nucleic acid molecule is antisense to a “noncoding region” of the coding strand of a nucleotide sequence of the invention. The term “noncoding region” refers to 5′ and 3′ sequences which flank the coding region that are not translated into amino acids (i.e., also referred to as 5′ and 3′ untranslated regions).

Given the coding strand sequences disclosed herein, antisense nucleic acids of the invention can be designed according to the rules of Watson and Crick or Hoogsteen base pairing. For example, the antisense nucleic acid molecule can generally be complementary to the entire coding region of an mRNA, but more preferably as embodied herein, it is an oligonucleotide that is antisense to only a portion of the coding or noncoding region of the mRNA. An antisense oligonucleotide can range in length between about 5 and about 60 nucleotides, preferably between about 10 and about 45 nucleotides, more preferably between about 15 and 40 nucleotides, and still more preferably between about 15 and 30 in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polymorphic protein to thereby inhibit expression of the protein, e.g., by inhibiting transcription and/or translation. The hybridization can be by conventional nucleotide complementary to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual -units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

The following terms are used to describe the sequence relationships between two or more nucleic acids or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Optimal alignment of sequences for aligning a comparison window may, for example, be conducted by the local homology algorithm of Smith and Waterman Adv. AppI. Math, 2482 (1981), by the homology alignment algorithm of Needleman and Wunsch J. Mol. Biol. 48:443 (1970), by the search for similarity method of Pearson and Lipman Proc. Natl. Acad. Sci. U.S.A. 852444 (1988), or by computerized implementations of these algorithms (for example, GAP, BESTFIT, FASTA, and TFASTA in the Wisconsin Genetics Software Package Release 7.0, Genetics Computer Group, 575 Science Dr., Madison, Wis.).

Techniques for nucleic acid manipulation of the nucleic acid sequences harboring the cSNP's of the invention, such as subcloning nucleic acid sequences encoding polypeptides into expression vectors, labeling probes, DNA hybridization, and the like, are described generally in Sambrook et al., The phrase “nucleic acid sequence encoding” refers to a nucleic acid which directs the expression of a specific protein, peptide or amino acid sequence. The nucleic acid sequences include both the DNA strand sequence that is transcribed into RNA and the RNA sequence that is translated into protein, peptide or amino acid sequence. The nucleic acid sequences include both the full length nucleic acid sequences disclosed herein as well as non-full length sequences derived from the full length protein. It being further understood that the sequence includes the degenerate codons of the native sequence or sequences which may be introduced to provide codon preference in a specific host cell. Consequently, the principles of probe selection and array design can readily be extended to analyze more complex polymorphisms (see EP 730,663). For example, to characterize a triallelic SNP polymorphism, three groups of probes can be designed tiled on the three polymorphic forms as described above. As a further example, to analyze a diallelic polymorphism involving a deletion of a nucleotide, one can tile a first group of probes based on the undeleted polymorphic form as the reference sequence and a second group of probes based on the deleted form as the reference sequence.

For assay of genomic DNA, virtually any biological convenient tissue samples include whole blood, semen, saliva, tears, urine, fecal material, sweat, buccal, skin and hair can be used. Genomic DNA is typically amplified before analysis. Amplification is usually effected by PCR using primers flanking a suitable fragment e.g., of 50-500 nucleotides containing the locus of the polymorphism to be analyzed. Target is usually labeled in the course of amplification. The amplification product can be RNA or DNA, single stranded or double stranded. If double stranded, the amplification product is typically denatured before application to an array. If genomic DNA is analyzed without amplification, it may be desirable to remove RNA from the sample before applying it to the array. Such can be accomplished by digestion with DNase-free RNAase.

### DETECTION OF POLYMORPHISMS IN A NUCLEIC ACID SAMPLE

The SNPs disclosed herein can be used to determine which forms of a characterized polymorphism are present in individuals under analysis.

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 7, 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

The polymorphisms can also be identified by hybridization to nucleic acid arrays, some examples of which are described in oublished PCT application WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

An allele-specific primer hybridizes to a site on target DNA overlapping a polymorphism and only primes amplification of an allelic form to which the primer exhibits perfect complementarity. See Gibbs, Nucleic Acid Res. 17 2427-2448 (1989). This primer is used in conjunction with a second primer which hybridizes at a distal site. Amplification proceeds from the two-primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co New York, 1992, Chapter 7).

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

The genotype of an individual with respect to a pathology suspected of being caused by a genetic polymorphism may be assessed by association analysis. Phenotypic traits suitable for association analysis include diseases that have known but hitherto unmapped genetic components (e.g., agammaglobulinemia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria).

Phenotypic traits also include symptoms of, or susceptibility to, multifactorial diseases of which a component is or may be genetic, such as autoimmune diseases, inflammation, cancer, system, diseases of the nervous and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, oral cavity, ovary, pancreas, prostate, skin, stomach, leukemia, liver, lung, and uterus. Phenotypic traits also include characteristics such as longevity, appearance (e.g., baldness, obesity), strength, speed, endurance, fertility, and susceptibility or receptivity to particular drugs or therapeutic treatments.

Such correlations can be exploited in several ways. In the case of a strong correlation between a polymorphic form and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. After determining polymorphic form(s) present in an individual at one or more polymorphic sites, this information can be used in a number of methods.

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, D.C., 1996). Since the polymorphic sites are within a 50,000 bp region in the human genome, the probability of recombination between these polymorphic sites is low. That low probability means the haplotype (the set of all 10 polymorphic sites) set forth in this application should be inherited without change for at least several generations. The more sites that are analyzed the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are diallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In diallelic loci, four genotypes are. possible: AA, AB, BA, and BB. If alleles A and B occur in a haploid genome of the organism with frequencies x and y, the probability of each genotype in a diploid organism are (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e, the probability that two individuals, picked at random from a population will have identical polymorphic forms at a given locus) is given by the equation:

p(ID)=(x²)²⁺(2xy)²⁺(y²)².

These calculations can be extended for any number of polymorphic forms at a given locus. For example, the probability of identity p(ID) for a 3-allele system where the alleles have the frequencies in the population of x, y and z, respectively, is equal to the sum of the squares of the genotype frequencies:

p(ID)=x⁴⁺(2xy)²⁺(2yz)²⁺(2xz)²⁺z⁴⁺y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus:

cum p(ID)=p(ID1)p(ID2)p(ID3) . . . p(IDn)

The cumulative probability of non-identity for n loci (i.e. the probability that two random individuals will be different at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the putative father, it can be concluded, barring experimental error, that the putative father is not the real father. If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WO 95/12607):

p(exc)=xy(1−xy)

where x and y are the population frequencies of alleles A and B of a diallelic polymorphic site. (At a triallelic site p(exc)=xy(1−xy)+yz(1−yz)+xz(1−xz)+3xyz(1−xyz))), where x, y and z and the respective population frequencies of alleles A, B and C). The probability of non-exclusion is:

p(non-exc)=1−p(exc)

The cumulative probability of non-exclusion (representing the value obtained when n loci are used) is thus:

cum p(non-exc)=p(non-exc1)p(non-exc2)p(non-exc3) . . . p(non-excn)

The cumulative probability of exclusion for n loci (representing the probability that a random male will be excluded) is:

cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components. Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

Correlation is performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods such as a -squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

For animals and plants, correlations between characteristics and phenotype are useful for breeding for desired characteristics. For example, Beitz et al., U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 if variant or 0 if wild type with respect to a prototypical mitochondrial DNA sequence at each of 17 locations considered.

The previous section concerns identifying correlations between phenotypic traits and polymorphisms that directly -or indirectly contribute to those traits. The present section describes identification of a physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992) (each of which is incorporated by reference in its entirety for all purposes).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction, versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions ( ), ranging from =0.0 (coincident loci) to =0.50 (unlinked). Thus, the likelihood at a given value of is: probability of data if loci linked at to probability of data if loci unlinked. The computed likelihood is usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of at which thelod score is the highest is considered to be the best estimate of the recombination fraction.

Positive lod score values suggest that the two loci are linked, whereas negative values suggest that linkage is less likely (at that value of) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. (1989). Inactivation of endogenous variant genes can be achieved by forming a transgene in which a cloned variant gene is inactivated by insertion of a positive selection marker. See Capecchi, Science 244, 1288-1292 The transgene is then introduced into an embryonic stem cell, where it undergoes homologous recombination with an endogenous variant gene. Mice and other rodents are preferred animals. Such animals provide useful drug screening systems.

The invention further provides methods for assessing the pharmacogenomic susceptibility of a subject harboring a single nucleotide polymorphism to a particular pharmaceutical compound, or to a class of such compounds. Genetic polymorphism in drug-metabolizing enzymes, drug transporters, receptors for pharmaceutical agents, and other drug targets have been correlated with individual differences based on distinction in the efficacy and toxicity of the pharmaceutical agent administered to a subject. Pharmocogenomic characterization of a subjects susceptibility to a drug enhances the ability to tailor a dosing regimen to the particular genetic constitution of the subject, thereby enhancing and optimizing the therapeutic effectiveness of the therapy.

In cases in which a cSNP leads to a polymorphic protein that is ascribed to be the cause of a pathological condition, method of treating such a condition includes administering to a subject experiencing the pathology the wild type cognate of the polymorphic protein. Once administered in an effective dosing regimen, the wild type cognate provides complementation or remediation of the defect due to the polymorphic protein. The subject's condition is ameliorated by this protein therapy.

A subject suspected of suffering from a pathology ascribable to a polymorphic protein that arises from a cSNP is to be diagnosed using any of a variety of diagnostic methods capable of identifying the presence of the cSNP in the nucleic acid, or of the cognate polymorphic protein, in a suitable clinical sample taken from the subject. Once the presence of the cSNP has been ascertained, and the pathology is correctable by administering a normal or wild-type gene, the subject is treated with a pharmaceutical composition that includes a nucleic acid that harbors the correcting wild-type gene, or a fragment containing a correcting sequence of the wild-type gene. Non-limiting examples of ways in which such a nucleic acid may be administered include incorporating the wild-type gene in a viral vector, such as an adenovirus or adeno associated virus, and administration of a naked DNA in a pharmaceutical composition that promotes intracellular uptake of the administered nucleic acid. Once the nucleic acid that includes the gene coding for the wild-type allele of the polymorphism is incorporated within a cell of the subject, it will initiate de novo biosynthesis of the wild-type gene product. If the nucleic acid is further incorporated into the genome of the subject, the treatment will have long-term effects, providing de novo synthesis of the wild-type protein for a prolonged duration. The synthesis of the wild-type protein in the cells of the subject will contribute to a therapeutic enhancement of the clinical condition of the subject.

A subject suffering from a pathology ascribed to a SNP may be treated so as to correct the genetic defect. (See Kren et al., Proc. Natl. Acad. Sci. USA 96:10349-10354 (1999)). Such a subject is identified by any method that can detect the polymorphism in a sample drawn from the subject. Such a genetic defect may be permanently corrected by administering to such a subject a nucleic acid fragment incorporating a repair sequence that supplies the wild-type nucleotide at the position of the SNP. This site-specific repair sequence encompasses an RNA/DNA oligonucleotide which operates to promote endogenous repair of a subject's genomic DNA. Upon administration in an appropriate vehicle, such as a complex with polyethylenimine or encapsulated in anionic liposomes, a genetic defect leading to an inborn pathology may be overcome, as the chimeric oligonucleotides induces incorporation of the wild-type sequence into the subject's genome. Upon incorporation, the wild-type gene product is expressed, and the replacement is propagated, thereby engendering a permanent repair.

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 100, 1000 or all of the polymorphisms shown in the Table. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the hybridizing methods.

Several aspects of the present invention rely on having available the polymorphic proteins encoded by the nucleic acids comprising a SNP of the inventions. There are various methods of isolating these nucleic acid sequences. For example, DNA is isolated from a genomic or cDNA library using labeled oligonucleotide probes having sequences complementary to the sequences disclosed herein.

Such probes can be used directly in hybridization assays. Alternatively probes can be designed for use in amplification techniques such as PCR.

To prepare a cDNA library, mRNA is isolated from tissue such as heart or pancreas, preferably a tissue wherein expression of the gene or gene family is likely to occur. cDNA is prepared from the mRNA and ligated into a recombinant vector. The vector is transfected into a recombinant host for propagation, screening and cloning. Methods for making and screening cDNA libraries are well known, See Gubler, U. and Hoffman, B. J. Gene 25:263-269 (1983) and Sambrook et al.

For a genomic library, for example, the DNA is extracted from tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro, as described in Sambrook, et al. Recombinant phage are analyzed by plaque hybridization as described in Benton and Davis, Science 196:180-1 82 (1977). Colony hybridization is carried out as generally described in M. Grunstein et al. Proc. Natl. Acad. Sci. USA. 72:3961-3965 (1975). DNA of interest is identified in either cDNA or genomic libraries by its ability to hybridize with nucleic acid probes, for example on Southern blots, and these DNA regions are isolated by standard methods familiar to those of skill in the art. See Sambrook, et al.

In PCR techniques, oligonucleotide primers complementary to the two 3′ borders of the DNA region to be amplified are synthesized. The polymerase chain reaction is then carried out using the two primers. See PCR Protocols: a Guide to Methods and Applications (Innis, M, Gelfand, D., Sninsky, J. and White, T., eds.), Academic Press, San Diego (1990). Primers can be selected to amplify the entire regions encoding a full-length sequence of interest or to amplify smaller DNA. segments as desired. PCR can be used in a variety of protocols to isolate cDNA's encoding a sequence of interest. In these protocols, appropriate primers and probes for amplifying DNA encoding a sequence of interest are generated from analysis of the DNA sequences listed herein. Once such regions are PCR-amplified, they can be sequenced and oligonucleotide probes can be prepared from the sequence.

Once DNA encoding a sequence comprising a cSNP is isolated and cloned, one can express the encoded polymorphic proteins in a variety of recombinantly engineered cells. It is expected that those of skill in the art are knowledgeable in the numerous expression systems available for expression of DNA encoding a sequence of interest. No attempt to describe in detail the various methods known for the expression of proteins in prokaryotes or eukaryotes is made here.

In brief summary, the expression of natural or synthetic nucleic acids encoding a sequence of interest will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain, initiation sequences, transcription and translation terminators, and promoters useful for regulation of the expression of a polynucleotide sequence of interest. To obtain high level expression of a cloned gene, it is desirable to construct expression plasmids which contain, at the minimum, a strong promoter to direct transcription, a ribosome binding site for translational initiation, and a transcription/translation terminator. The expression vectors may also comprise generic expression cassettes containing at least one independent terminator sequence, sequences permitting replication of the plasmid in both eukaryotes and prokaryotes. i.e., shuttle vectors, and selection markers for both prokaryotic and eukaryotic systems. See Sambrook et al.

A variety of prokaryotic expression systems may be used to express the polymorphic proteins of the invention. Examples include E. coli, Bacillus, Streptomyces, and the like.

It is preferred to construct expression plasmids which contain, at the minimum, a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, C., J. Bacterial. 158:1018-1024 (1984) and the leftward promoter of phage lambda (P) as described by Λ, I. and Hagen, D., Ann. Rev. Genet. 14:399-445 (1980). The inclusion of selection markers in DNA vectors transformed in E. coli is also useful. Examples of such markers include genes specifying resistance to ampicillin, tetracycline, or chloramphenicol. See Sambrook et al. for details concerning selection markers for use in E. coli.

To enhance proper folding of the expressed recombinant protein, during purification from E. coli, the expressed protein may first be denatured and then renatured. This can be accomplished by solubilizing the bacterially produced proteins in a chaotropic agent such as guanidine HCl and reducing all the cysteine residues with a reducing agent such as beta-mercaptoethanol. The protein is then renatured, either by slow dialysis or by gel filtration. See U.S. Pat. No. 4,511,503. Detection of the expressed antigen is achieved by methods known in the art as radioimmunoassay, or Western blotting techniques or immunoprecipitation. Purification from E. coli can be achieved following procedures such as those described in U.S. Pat. No. 4,511,503.

Any of a variety of eukaryotic expression systems such as yeast, insect cell lines, bird, fish, and mammalian cells, may also be used to express a polymorphic protein of the invention. As explained briefly below, a nucleotide sequence harboring a cSNP may be expressed in these eukaryotic systems. Synthesis of heterologous proteins in yeast is well known. Methods in Yeast Genetics, Sherman, F., et al., Cold Spring Harbor Laboratory, (1982) is a well recognized work describing the various methods available to produce the protein in yeast. Suitable vectors usually have expression control sequences, such as promoters, including 3-phosphogtycerate kinase or other glycolytic enzymes, and an origin of replication, termination sequences and the like as desired. For instance, suitable vectors are described in the literature (Botstein, et al., Gene 8:17-24 (1979); Broach, et al., Gene 8:121-133 (1979)).

Two procedures are used in transforming yeast cells. In one case, yeast cells are first converted into protoplasts using zymolyase, lyticase or glusulase, followed by addition of DNA and polyethylene glycol (PEG). The PEG-treated protoplasts are then regenerated in a 3% agar medium under selective conditions. Details of this procedure are given in the papers by J. D. Beggs, Nature (London) 275:104-109 (1978); and Hinnen, A., et al., Proc. Natl. Acad. Sci. USA, 75:1929-1933 (1978). The second procedure does not involve removal of the cell wall. Instead the cells are treated with lithium chloride or acetate and PEG and put on selective plates (Ito, H., et al., J. Bact, 153163-168 (1983)). cells and applying standard protein isolation techniques to the lysates:.

The purification process can be monitored by using Western blot techniques or radioimmunoassay or other standard techniques. The sequences encoding the proteins of the invention can also be ligated to various immunoassay expression vectors for use in transforming cell cultures of, for instance, mammalian, insect, bird or fish origin. Illustrative of cell cultures useful for the production of the polypeptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines, and various human cells such as COS cell lines, HeLa cells, myeloma cell lines, Jurkat cells, etc. Expression vectors for these cells can include expression control sequences, such as an origin of replication, a promoter (e.g., the CMV promoter, a HSV tk promoter or pgk (phosphoglycerate kinase) promoter), an enhancer (Queen et al. Immunol. Rev. 89:49 (1986)) and necessary processing information sites, such as ribosome binding sites, RNA splice sites, polyadenylation sites (e.g., an SV40 large T Ag poly A addition site), and transcriptional terminator sequences.

Other animal cells are available, for instance, from the American Type Culture Collection Catalogue of Cell Lines and Hybridomas (7th edition, (1992)). Appropriate vectors for expressing the proteins of the invention in insect cells are usually derived from baculovirus. Insect cell lines include mosquito larvae, silkworm, armyworm, moth and Drosophila cell lines such as a Schneider cell line (See Schneider J. Embryol. Exp. Morphol., 27:353-365 (1987). As indicated above, the vector, e.g., a plasmid, which is used to transform the host cell, preferably contains DNA sequences to initiate transcription and sequences to control the translation of the protein. These sequences are referred to as expression control sequences. As with yeast, when higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, J. et a/., J. Virol. 45: 773-781 (1983)). Additionally, gene sequences to control replication in the host cell may be Saveria-Campo, M., 1985, “Bovine Papilloma virus DNA a Eukaryotic Cloning Vector” in DNA Cloning Vol. II a Practical Approach Ed. D. M. Glover, IRL Press, Arlington, Va. pp. 213-238. The host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation and micro-injection of the DNA directly into the cells.

The transformed cells are cultured by means well known in the art (Biochemical Methods in Cell Culture and Virology, Kuchler, R. J., Dowden, Hutchinson and Ross, Inc., (1977)). The expressed polypeptides are isolated from cells grown as suspensions or as monolayers. The latter are recovered by well known mechanical, chemical or enzymatic means.

General methods of expressing recombinant proteins are also known and are exemplified in R. Kaufman, Methods in Enzymology 185, 537-566 (1990). As defined herein “operably linked” refers to linkage of a promoter upstream from a DNA sequence such that the promoter mediates transcription of the DNA sequence. Specifically, “operably linked” means that the isolated polynucleotide of the invention and an expression control sequence are situated within a vector or cell in such a way that the gene encoding the protein is expressed by a host cell which has been transformed (transfected) with the ligated polynucleotide/expression sequence. The term “vector”, refers to viral expression systems, autonomous self-replicating circular DNA (plasmids), and includes both expression and nonexpression plasmids.

The term “gene” as used herein is intended to refer to a nucleic acid sequence which encodes a polypeptide. This definition includes various sequence polymorphisms, mutations, and/or sequence variants wherein such alterations do not affect the function of the gene product. The term “gene” is intended to include not only coding sequences but also regulatory regions such as promoters, enhancers, termination regions and similar untranslated nucleotide sequences. The term further includes all introns and other DNA sequences spliced from the mRNA transcript, along with variants resulting from alternative splice sites.

A number of types of cells may act as suitable host cells for expression of the protein. Mammalian host cells include, for example, monkey COS cells, Chinese Hamster Ovary (CHO) cells, human kidney 293 cells, human epidermal A43 1 cells, human Co10205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from. e.g., Invitrogen, San Diego, Calif., U.S.A. (the MaxBac© kit), and such methods are well known in the art, as described in Summers and Smith, Texas Agricultural Experiment Station Bulletin No. 1555 (1987). incorporated herein by reference. As used herein, an insect cell capable of expressing_a polynucleotide of the present invention is “transformed.” The protein of the invention may be prepared by culturing transformed host cells under culture conditions suitable to express the recombinant protein.

The polymorphic protein of the invention may also be expressed as a product of transgenic animals, e.g., as a component of the milk of transgenic cows, goats, pigs, or sheep which are characterized by somatic or germ cells containing a nucleotide sequence encoding the protein. The protein may also be produced by known conventional chemical synthesis. Methods for constructing the proteins of the present invention by synthetic means are known to those skilled in the art.

The polymorphic proteins produced by recombinant DNA technology may be purified by techniques commonly employed to isolate or purify recombinant proteins. Recombinantly produced proteins can be directly expressed or expressed as a fusion protein. The protein is then purified by a combination of cell lysis (e.g., sonication) and affinity chromatography. For fusion products, subsequent digestion of the fusion protein with an appropriate proteolytic enzyme releases the desired polypeptide. The polypeptides of this invention may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982), incorporated herein by reference. For example, in an embodiment, antibodies may be raised to the proteins of the invention as described herein. Cell membranes are isolated from a cell line expressing the recombinant protein, the protein is extracted from the membranes and immunoprecipitated. The proteins may then be further purified by standard protein chemistry techniques as described above.

The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-Toyopearl@ or Cibacrom blue 3GA Sepharose B; one or more steps involving hydrophobic interaction chromatography using such resins as phenyl ether, butyl ether, or propyl ether; or immunoaffinity chromatography. Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The term “antibody” as used herein refers to immunoglobulin molecules and immunologically active portions of immunoglobulin molecules, i.e., molecules that contain an antigen binding site that specifically binds (immunoreacts with) an antigen, such as polymorphic. Such antibodies include, but are not limited to, polyclonal, monoclonal, chimeric, single chain, F_{ab} and F_{(ab′)2} fragments, and an F_{ab} expression library. In a specific embodiment, antibodies to human polymorphic proteins are disclosed.

The phrase “specifically binds to”, “immunospecifically binds to” or is “specifically immunoreactive with”, an antibody when referring to a protein or peptide, refers to a binding reaction which is determinative of the presence of the protein in the presence of a heterogeneous population of proteins and other biological materials. Thus, for example, under designated immunoassay conditions, the specified antibodies bind to a particular protein and do not bind in a significant amount to other proteins present in the sample. Specific binding to an antibody under such conditions may require an antibody that is selected for its specificity for a particular protein. Of particular interest in the present invention is an antibody that binds immunospecifically to a polymorphic protein but not to its cognate wild type allelic protein, or vice versa. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, a Laboratory Manual. Cold Spring Harbor Publications, New York, for a description of immunoassay formats and conditions that can be used to determine specific immunoreactivity.

Polyclonal and/or monoclonal antibodies that immunospecifically bind to polymorphic gene products but not to the corresponding prototypical or “wild-type” gene products are also provided. Antibodies can be made by injecting mice or other animals with the variant gene product or synthetic peptide. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product.

An isolated polymorphic protein, or a portion or fragment thereof, can be used as an immunogen to generate the antibody that bind the polymorphic protein using standard techniques for polyclonal and monoclonal antibody preparation. The full-length polymorphic protein can be used or, alternatively, the invention provides antigenic peptide fragments of polymorphic for use as immunogens. The antigenic peptide of a polymorphic protein of the invention comprises at least 8 amino acid residues of the amino acid sequence encompassing the polymorphic amino acid and encompasses an epitope of the polymorphic protein such that an antibody raised against the peptide forms a specific immune complex with the polymorphic protein. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of polymorphic that are located on the surface of the protein, e.g., hydrophilic regions.

For the production of polyclonal antibodies, various suitable host animals (e.g., rabbit, goat, mouse or other mammal) may be immunized by injection with the polymorphic protein. An appropriate immunogenic preparation can contain, for example, recombinantly expressed polymorphic protein or a chemically synthesized polymorphic polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.). human adjuvants such as Bacille Calmette-Guerin and Corynebacterium parvum, or similar immunostimulatory agents. If desired, the antibody molecules directed against polymorphic proteins can be isolated from the mammal (e.g., from the blood) and further purified by well known techniques, such as protein A chromatography, to obtain the IgG fraction.

The term “monoclonal antibody” or “monoclonal antibody composition”, as used herein, refers to a population of antibody molecules that originates from the clone of a singly hybridoma cell, and that contains only one type of antigen binding site capable of immunoreacting with a particular epitope of a polymorphic protein. A monoclonal antibody composition thus typically displays a single binding affinity for a particular polymorphic protein with which it immunoreacts. For preparation of monoclonal antibodies directed towards a particular polymorphic protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to a polymorphic protein (see e.g., U.S. Pat. No. 4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a polymorphic protein or derivatives, fragments, analogs or homologs thereof. Non-human antibodies can be “humanized” by techniques well known in the art. See e.g., U.S. Pat. No. 5,225,539. Antibody fragments that contain the idiotypes to a polymorphic protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-polymorphic protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methodologies for the screening of antibodies that possess the desired specificity include, but are not limited to, enzyme-linked immunosorbent assay (ELISA) and other immunologically-mediated techniques known within the art.

Anti-polymorphic protein antibodies may be used in methods known within the art relating to the detection, quantitation and/or cellular or tissue localization of a polymorphic protein (e.g., for use in measuring levels of the polymorphic protein within appropriate physiological samples, for use in diagnostic methods, for use in imaging the protein, and the like). In a given embodiment, antibodies for polymorphic proteins, or derivatives, fragments, analogs or homologs thereof, that contain the antibody-derived CDR, are utilized as pharmacologically-active compounds in therapeutic applications intended to treat a pathology in a subject that arises from the presence of the cSNP allele in the subject.

An anti-polymorphic protein antibody (e.g., monoclonal antibody) can be used to isolate polymorphic proteins by a variety of immunochemical techniques, such as immunoaffinity chromatography or immunoprecipitation. An anti-polymorphic protein antibody can facilitate the purification of natural polymorphic protein from cells and of recombinantly produced polymorphic proteins expressed in host cells. Moreover, an anti-polymorphic protein antibody can be used to detect polymorphic protein (e.g., in a cellular lysate or cell supernatant) in order to evaluate the abundance and pattern of expression of the polymorphic protein. Anti-polymorphic antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

What is claimed is:

1. An isolated polynucleotide selected from the group consisting of:
a) a nucleotide sequence comprising one or more polymorphic sequences (SEQ ID NOS:1-651);
b) a fragment of said nucleotide sequence, provided that the fragment includes a polymorphic site in said polymorphic sequence;
c) a complementary nucleotide sequence comprising a sequence complementary to one or more of said polymorphic sequences (SEQ ID NOS:1-651); and
d) a fragment of said complementary nucleotide sequence, provided that the fragment includes a polymorphic site in said polymorphic sequence.

2. The polynucleotide of claim 1, wherein said polynucleotide sequence is DNA.

3. The polynucleotide of claim 1, wherein said polynucleotide sequence is RNA.

4. The polynucleotide of claim 1, wherein said polynucleotide sequence is between about 10 and about 100 nucleotides in length.

5. The polynucleotide of claim 1, wherein said polynucleotide sequence is between about 10 and about 90 nucleotides in length.

6. The polynucleotide of claim 1, wherein said polynucleotide sequence is between about 10 and about 75 nucleotides in length.

7. The polynucleotide of claim 1, wherein said polynucleotide is between about 10 and about 50 bases in length.

8. The polynucleotide of claim 1, wherein said polynucleotide is between about 10 and about 40 bases in length.

9. The polynucleotide of claim 1, wherein said polynucleotide is derived from a nucleic acid encoding a polypeptide related to angiopoietin, 4-hydroxybutyrate dehydrogenase, ATP-dependent RNA helicase, MHC Class I histocompatibility antigen, or phosphoglycerate kinase.

10. The polynucleotide of claim 1, wherein said polymorphic site includes a nucleotide other than the nucleotide listed in Table 1, column 5 for said polymorphic sequence.

11. The polynucleotide of claim 1, wherein the complement of said polymorphic site includes a nucleotide other than the complement of the nucleotide listed in Table 1, column 5 for the complement of said polymorphic sequence.

12. The polynucleotide of claim 1, wherein said polymorphic site includes the nucleotide listed in Table 1, column 6 for said polymorphic sequence.

13. The polynucleotide of claim 1, wherein the complement of said polymorphic site includes the complement of the nucleotide listed in Table 1, column 6 for said polymorphic sequence.

14. An isolated allele-specific oligonucleotide that hybridizes to a first polynucleotide at a polymorphic site encompassed therein, wherein the first polynucleotide is chosen from the group consisting of:
a) a nucleotide sequence comprising one or more polymorphic sequences (SEQ ID NOS:1-651) provided that the polymorphic sequence includes a nucleotide other than the nucleotide recited in Table 1, column 5 for said polymorphic sequence;
b) a nucleotide sequence that is a fragment of said polymorphic sequence, provided that the fragment includes a polymorphic site in said polymorphic sequence;
c) a complementary nucleotide sequence comprising a sequence complementary to one or more polymorphic sequences (SEQ ID NOS:1-651), provided that the complementary nucleotide sequence includes a nucleotide other than the complement of the nucleotide recited in Table 1, column 5; and
d) a nucleotide sequence that is a fragment of said complementary sequence, provided that the fragment includes a polymorphic site in said polymorphic sequence.

15. The oligonucleotide of claim 14, wherein the oligonucleotide does not hybridize under stringent conditions to a second polynucleotide selected from the group consisting of:
a) a nucleotide sequence comprising one or more polymorphic sequences (SEQ ID NOS:1-651), wherein said polymorphic sequence includes the nucleotide listed in Table 1, column 5 for said polymorphic sequence;
b) a nucleotide sequence that is a fragment of any of said nucleotide sequences;
c) a complementary nucleotide sequence comprising a sequence complementary to one or more polymorphic sequences (SEQ ID NOS:1-651), wherein said polymorphic sequence includes the complement of the nucleotide listed in Table 1, column 5; and
d) a nucleotide sequence that is a fragment of said complementary sequence, provided that the fragment includes a polymorphic site in said polymorphic sequence.

16. The oligonucleotide of claim 15, wherein the oligonucleotide is between about 10 and about 51 bases in length.

17. The oligonucleotide of claim 15, wherein the oligonucleotide identifies a polypeptide related to angiopoietin, 4-hydroxybutyrate dehydrogenase, ATP-dependent RNA helicase, MHC Class I histocompatibility antigen, or phosphoglycerate kinase.

18. The oligonucleotide of claim 15, wherein the oligonucleotide is between about 15 and about 30 bases in length.

19. A method of detecting a polymorphic site in a nucleic acid, the method comprising:
a) contacting said nucleic acid with an oligonucleotide that hybridizes to a polymorphic sequence selected from the group consisting of SEQ ID NOS: 1-651, or its complement, provided that the polymorphic sequence includes a nucleotide other than the nucleotide recited in Table 1, column 5 for said polymorphic sequence, or the complement includes a nucleotide other than the complement of the nucleotide recited in Table 1, column 5; and
b) determining whether said nucleic acid and said oligonucleotide hybridize;
whereby hybridization of said oligonucleotide to said nucleic acid sequence indicates the presence of the polymorphic site in said nucleic acid.

20. The method of claim 19, wherein said oligonucleotide does not hybridize to said polymorphic sequence when said polymorphic sequence includes the nucleotide recited in Table 1, column 5 for said polymorphic sequence, or when the complement of the polymorphic sequence includes the complement of the nucleotide recited in Table 1, column 5 for said polymorphic sequence.

21. The method of claim 19, wherein said oligonucleotide identifies a polypeptide related to angiopoietin, 4-hydroxybutyrate dehydrogenase, ATP-dependent RNA helicase, MHC Class I histocompatibility antigen, or phosphoglycerate kinase.

22. The method of claim 19, wherein said oligonucleotide is between about 15 and about 30 bases in length.

23. A method of detecting the presence of a sequence polymorphism in a subject, the method comprising:
a) providing a nucleic acid from said subject;
b) contacting said nucleic acid with an oligonucleotide that hybridizes to a polymorphic sequence selected from the group consisting of SEQ ID NOS:1-651, or its complement, provided that the polymorphic sequence includes a nucleotide other than the nucleotide recited in for said polymorphic sequence, or the complement includes a nucleotide other than the complement of the nucleotide recited in Table 1, column 5; and
c) determining whether said nucleic acid and said oligonucleotide hybridize;
whereby hybridization of said oligonucleotide to said nucleic acid sequence indicates the presence of the polymorphism in said subject.

24. A method of determining the relatedness of a first and second nucleic acid, the method comprising:
a) providing a first nucleic acid and a second nucleic acid;
b) contacting said first nucleic acid and said second nucleic acid with an oligonucleotide that hybridizes to a polymorphic sequence selected from the group consisting of SEQ ID NOS:1-651, or its complement, provided that the polymorphic sequence includes a nucleotide other than the nucleotide recited in Table 1, column 5 for said polymorphic sequence, or the complement includes a nucleotide other than the complement of the nucleotide recited in Table 1, column 5;
c) determining whether said first nucleic acid and said second nucleic acid hybridize to said oligonucleotide; and
d) comparing hybridization of said first and second nucleic acids to said oligonucleotide,
wherein hybridization of the first and second nucleic acids to said oligonucleotide indicates the first and second nucleic acids are related.

25. The method of claim 24, wherein said oligonucleotide does not hybridize to said polymorphic sequence when said polymorphic sequence includes the nucleotide recited in Table 1, column 5 for said polymorphic sequence, or when the complement of the polymorphic sequence includes the complement of the nucleotide recited in Table 1, column 5 for said polymorphic sequence.

26. The method of claim 24, wherein the oligonucleotide is between about 10 and about 51 bases in length.

27. The method of claim 24, wherein the oligonucleotide is between about 10 and about 40 bases in length.

28. The method of claim 24, wherein the oligonucleotide is between about 15 and about 30 bases in length.

29. An isolated polypeptide comprising a polymorphic site at one or more amino acid residues, wherein the protein is encoded by a polynucleotide selected from the group consisting of: polymorphic sequences SEQ ID NOS:1-651, or their complement, provided that the polymorphic sequence includes a nucleotide other than the nucleotide recited in Table 1, column 5 for said polymorphic sequence, or the complement includes a nucleotide other than the complement of the nucleotide recited in Table 1, column 5.

30. The polypeptide of claim 29, wherein said polypeptide is translated in the same open reading frame as is a wild type protein whose amino acid sequence is identical to the amino acid sequence of the polymorphic protein except at the site of the polymorphism.

31. The polypeptide of claim 29, wherein the polypeptide encoded by said polymorphic sequence, or its complement, includes the nucleotide listed in Table 2, column 6 or Table 3, column 5 for said polymorphic sequence, or the complement includes the complement of the nucleotide listed in Table 1, column 6.

32. An antibody that binds specifically to a polypeptide encoded by a polynucleotide comprising a nucleotide sequence encoded by a polynucleotide selected from the group consisting of polymorphic sequences SEQ ID NOS:1-651, or its complement, provided that the polymorphic sequence includes a nucleotide other than the nucleotide recited in Table 1, column 5 for said polymorphic sequence, or the complement includes a nucleotide other than the complement of the nucleotide recited in Table 1, column 5.

33. The antibody of claim 32, wherein said antibody binds specifically to a polypeptide encoded by a polymorphic sequence which includes the nucleotide listed in Table 1, column 6 for said polymorphic sequence.

34. The antibody of claim 32, wherein said antibody does not bind specifically to a polypeptide encoded by a polymorphic sequence which includes the nucleotide listed in Table 1, column 5 for said polymorphic sequence.

35. A method of detecting the presence of a polypeptide having one or more amino acid residue polymorphisms in a subject, the method comprising
a) providing a protein sample from said subject;
b) contacting said sample with the antibody of claim 34 under conditions that allow for the formation of antibody-antigen complexes; and
c) detecting said antibody-antigen complexes,
whereby the presence of said complexes indicates the presence of said polypeptide.

36. A method of treating a subject suffering from, at risk for, or suspected of, suffering from a pathology ascribed to the presence of a sequence polymorphism in a subject, the method comprising:
a) providing a subject suffering from a pathology associated with aberrant expression of a first nucleic acid comprising a polymorphic sequence selected from the group consisting of SEQ ID NOS:1-651, or its complement; and
b) administering to the subject an effective therapeutic dose of a second nucleic acid comprising the polymorphic sequence, provided that the second nucleic acid comprises the nucleotide present in a wild type allele of the sequence polymorphism,
thereby treating said subject.

37. The method of claim 36, wherein the second nucleic acid sequence comprises a polymorphic sequence which includes the nucleotide listed in Table 1, column 5 for said polymorphic sequence.

38. A method of treating a subject suffering from, at risk for, or suspected of suffering from a pathology ascribed to the presence of a sequence polymorphism in a subject, the method comprising:
a) providing a subject suffering from a pathology associated with aberrant expression of a polymorphic sequence selected from the group consisting of polymorphic sequences SEQ ID NOS:1-651, or its complement; and
b) administering to the subject an effective therapeutic dose of a polypeptide,
wherein said polypeptide is encoded by a polynucleotide comprising a polymorphic sequence selected from the group consisting of SEQ ID NOS:1-651, or by a polynucleotide comprising a nucleotide sequence that is complementary to any one of polymorphic sequences SEQ ID NOS:1-651, provided that said polymorphic sequence includes the nucleotide listed in Table 1, column 6 for said polymorphic sequence, thereby treating said subject.

39. A method of treating a subject suffering from, at risk for, or suspected of suffering from, a pathology ascribed to the presence of a sequence polymorphism in a subject, the method comprising:
a) providing a subject suffering from, at risk for, or suspected of suffering from, a pathology associated with aberrant expression of a first nucleic acid comprising a polymorphic sequence selected from the group consisting of SEQ ID NOS:1-651, or its complement; and
b) administering to the subject an effective dose of the antibody of claim 34,
thereby treating said subject.

40. A method of treating a subject suffering from, at risk for, or suspected of suffering from, a pathology ascribed to the presence of a sequence polymorphism in a subject, the method comprising:
a) providing a subject suffering from, at risk for, or suspected of suffering from, a pathology associated with aberrant expression of a nucleic acid comprising a polymorphic sequence selected from the group consisting of SEQ ID NOS:1-651, or its complement; and
b) administering to the subject an effective dose of an oligonucleotide comprising a polymorphic sequence selected from the group consisting of SEQ ID NOS:1-651, or by a polynucleotide comprising a nucleotide sequence that is complementary to any one of polymorphic sequences SEQ ID NOS:1-651, provided that said polymorphic sequence includes the nucleotide listed in Table 1, column 6 for said polymorphic sequence,
thereby treating said subject.

41. An oligonucleotide array, comprising one or more oligonucleotides hybridizing to a first polynucleotide at a polymorphic site encompassed therein, wherein the first polynucleotide is chosen from the group consisting of:
a) a nucleotide sequence comprising one or more polymorphic sequences SEQ ID NOS:1-651;
b) a nucleotide sequence that is a fragment of any of said nucleotide sequence, provided that the fragment includes a polymorphic site in said polymorphic sequence;
c) a complementary nucleotide sequence comprising a sequence complementary to one or more polymorphic sequences SEQ ID NOS:1-651; and
d) a nucleotide sequence that is a fragment of said complementary sequence, provided that the fragment includes a polymorphic site in said polymorphic sequence.

42. The array of claim 41, wherein said array comprises 10 oligonucleotides.

43. The array of claim 41, wherein said array comprises at least 100 oligonucleotides.

44. The array of claim 41, wherein said array comprises at least 1000 oligonucleotides.
